(19) United States
(12) Patent Application Publication
(10) Pub. No.: US 2022/0000896 A1
(43) Pub. Date: Jan. 6, 2022

(54) METHODS OF TREATMENT ASSOCIATED WITH ENDOVASULAR GRAFTS

(71) Applicant: Nectero Medical, Inc., Tempe, AZ (US)

(72) Inventor: Kelvin Ning, Scottsdale, AZ (US)

(21) Appl. No.: 17/448,318

(22) Filed: Sep. 21, 2021

Related U.S. Application Data
(63) Continuation of application No. PCT/US2020/024739, filed on Mar. 25, 2020.
(60) Provisional application No. 62/824,091, filed on Mar. 26, 2019.
(60) Provisional application No. 62/824,016, filed on Mar. 26, 2019.
(60) Provisional application No. 62/824,167, filed on Mar. 26, 2019.
(60) Provisional application No. 62/824,100, filed on Mar. 26, 2019.

(51) Int. Cl. A61K 31/7034; A61M 25/10; A61L 31/06; A61L 31/16

(52) U.S. Cl. CPC A61K 31/7034; A61M 25/1011; A61F 2/07; A61L 31/16; A61L 31/06

(57) ABSTRACT

Methods and compositions for treatments associated with endovascular grafts, dissections, peripheral aneurysms, and neuro aneurysms are provided that deliver 1,2,3,4,6-pentagalloyl glucose (PGG) or analogues or derivatives thereof and/or LeGoo®. Also provided is a device to deliver 1,2,3,4,6-pentagalloyl glucose (PGG) or analogues or derivatives thereof or LeGoo® to the tissue to be treated.

### RELATED APPLICATIONS

This application is a continuation of International PCT Application No. PCT.US2020/024739 filed Mar. 25, 2020, which claims priority to each of U.S. Provisional Application No. 62/824,091 filed Mar. 26, 2019; U.S. Provisional Application No. 62/824,016 filed Mar. 26, 2019; U.S. Provisional Application No. 62/824,100 filed Mar. 26, 2019; and U.S. Provisional Application No. 62/824,167 filed March 26, 2019; the entire contents of each of which are hereby incorporated by reference herein.

### FIELD

Methods and compositions for treatments associated with endovascular grafts, dissections, peripheral aneurysms, and neuro aneurysms are provided that deliver 1,2,3,4,6-pentagalloyl glucose (PGG) or analogues or derivatives thereof and/or LeGoo®. Also provided is a device to deliver 1,2,3,4,6-pentagalloyl glucose (PGG) or analogues or derivatives thereof or LeGoo® to the tissue to be treated.

### BACKGROUND

An aortic aneurysm is an abnormal bulge that occurs in the wall of the major blood vessel (aorta) that carries blood from the heart to your body. Aortic aneurysms can occur anywhere in the aorta and may be tube-shaped (fusiform) or round (saccular). Types of aortic aneurysms include: abdominal aortic aneurysm, which occurs along the part of the aorta that passes through the abdomen, or thoracic aortic aneurysm, which occurs along the part of the aorta that passes through the chest cavity. In some cases, an individual may have an abdominal aortic aneurysm and a thoracic aortic aneurysm. Having an aortic aneurysm increases a risk of developing an aortic dissection, which occurs when a tear develops in the inner layer of the wall of the aorta. This causes one or more of the layers of the wall of the aorta to separate, which weakens the wall of the aorta.

The primary risk for an untreated aortic aneurysm is rupture, and as an aneurysm gets bigger, the risk gets greater. There are several factors to consider when deciding to treat an aneurysm with surgery, including: the presence of symptoms, including abdominal pain, back pain or pain in the groin or inner thigh; the size (or diameter) of the aneurysm; the rate of aneurysm growth; the development of an of an aortic dissection, which can be accompanied by sudden and severe sharp tearing pain in the chest or back; and the patient's overall medical condition.

Endovascular grafting is a minimally invasive method to treat an aortic aneurysm. Instead of an open aneurysm repair in which the patient's chest and abdomen are surgically opened, the surgeon may consider a procedure called an endovascular aneurysm repair (EVAR). The procedure is also referred to as a thoracic endovascular aneurysm repair (TEVAR) or fenestrated endovascular aneurysm Repair (FEVAR), depending on the type of repair being discussed. Endovascular surgery is performed inside the patient's aorta using catheters to place an endovascular surrounded with a fabric liner to reinforce weak spots in the vasculature. Patients may be eligible for endovascular grafting if the aneurysm has not ruptured and the aneurysm is 5 centimeters or more in size.

Aortic dissection is a serious condition in which there is a tear in the wall of the major artery carrying blood out of the heart (aorta). As the tear extends along the wall of the aorta, blood can flow in between the layers of the blood vessel wall (dissection). This can lead to aortic rupture or decreased blood flow (ischemia) to organs. When it leaves the heart, the aorta first moves up through the chest towards the head (the ascending aorta). It then bends or arches, and finally moves down through the chest and abdomen (the descending aorta).

Aortic dissection most often happens because of a tear or damage to the inner wall of the aorta. This very often occurs in the chest (thoracic) part of the artery, but it may also occur in the abdominal aorta. When a tear occurs, it creates two channels: one in which blood continues to travel and another where blood stays still. If the channel with non-traveling blood gets bigger, it can push on other branches of the aorta. This can narrow the other branches and reduce blood flow through them. An aortic dissection may also cause abnormal widening or ballooning of the aorta (aneurysm).

The exact cause is unknown, but more common risks include: aging, atherosclerosis, blunt trauma to the chest, such as hitting the steering wheel of a car during an accident, and high blood pressure. Other risk factors and conditions linked to aortic dissection include: bicuspid aortic valve, coarctation (narrowing) of the aorta, connective tissue disorders (such as Marfan syndrome and Ehlers-Danlos syndrome) and rare genetic disorders, heart surgery or procedures, pregnancy, and swelling of the blood vessels due to conditions such as arteritis and syphilis. Aortic dissection occurs in about 2 out of every 10,000 people. It can affect anyone, but is most often seen in men ages 40 to 70.

An aortic dissection is a medical emergency requiring immediate treatment. Therapy may include surgery or medications, depending on the area of the aorta involved.

Treatment for type A aortic dissection may include surgery, where surgeons remove as much of the dissected aorta as possible, block the entry of blood into the aortic wall and reconstruct the aorta with a synthetic tube called a graft. If the aortic valve leaks as a result of the damaged aorta, it may be replaced at the same time. The new valve is placed within the graft used to reconstruct the aorta. Treatment for type A aortic dissection may also include medications, such as beta blockers and nitroprusside (Nitropress), which reduce heart rate and lower blood pressure, and which can prevent the aortic dissection from worsening. They may be given to people with type A aortic dissection to stabilize blood pressure before surgery.

Treatment of type B aortic dissection may include the same medications used to treat type A aortic dissection. Treatment for type B aortic dissection may also include surgery. The procedure is similar to that used to correct a type A aortic dissection. Sometimes stents—small wire mesh tubes that act as a sort of scaffolding—may be placed in the aorta to repair complicated type B aortic dissections. After treatment, blood pressure lowering medication may be prescribed. Periodic monitoring and follow-up CTs or MRIs are also used.

One of the most common results of the degradation of vasculature is aneurysm. By definition, the term “aneurysm” is simply an abnormal widening or ballooning at the wall of a blood vessel. Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the iliac, popliteal and femoral arteries are prevalent locations of this vascular pathology. The occurrence of such peripheral aneurysms appears to be strongly associated with the presence of aneurysms in other locations, as it has been estimated that 30 to 60% of peripheral aneurysm patients also have an AAA.

Aneurysms can be devastating due to the potential for rupture or dissection that can lead to massive bleeding, stroke, or hemorrhagic shock, and can be fatal in an estimated 80% of cases. Aneurysms can be caused by any of a large class of degenerative diseases and pathologies including atherosclerotic disease, defects in arterial components, genetic susceptibilities, and high blood pressure, among others, and can develop silently over a period of years. The hallmarks of aneurysms include enzymatic degradation of vascular structural proteins such as elastin, collagen, inflammatory infiltrates, calcification, and eventual overall destruction of the vascular architecture. Elastin content in an aneurysmal aorta can be greatly reduced (for example, 70% less) than that of a healthy, undamaged aorta.

Aneurysms grow over a period of years and pose great risks to health. Aneurysms have the potential to dissect or rupture, causing massive bleeding, stroke, and hemorrhagic shock, which can be fatal in more than 80% of cases. AAAs are a serious health concern, specifically for the aging population, being among the top ten causes of death for patients older than 50. The estimated incidence for abdominal aortic aneurysm is about 50 in every 100,000 persons per year. Approximately 50,000 operations are performed each year in the U.S. for AAAs alone. In children, AAAs can result from blunt abdominal injury or from Marfan's syndrome, a defect in elastic fiber formation in walls of major arteries, such as the aorta.

Current methods of treatment for diagnosed aneurysms are limited to invasive surgical techniques. After initial diagnosis of a small aneurysm, the most common medical approach is to follow up the development of the aneurysm and after reaching a pre-determined size (for example, about 5 cm in diameter), surgical treatment is applied. Current surgical treatments are limited to either an endovascular stent graft repair or optionally complete replacement of the diseased vessel with a vascular graft. While such surgical treatments can save lives and improve quality of life for those suffering aneurysms, dangers beyond those of the surgery itself still exist for the patient due to possible post-surgery complications (for example, neurological injuries, bleeding, or stroke) as well as device-related complications (for example, thrombosis, leakage, or failure). Moreover, depending upon the location of the aneurysm, the danger of an invasive surgical procedure may outweigh the possible benefits of the procedure, for instance in the case of an aneurysm deep in the brain, leaving the sufferer with very little in the way of treatment options. Moreover, surgical treatments may not always provide a permanent solution, as vascular grafts can loosen and dislodge should the aneurysm progress following the corrective surgery. For some patients, the particular nature of the aneurysm or the condition of the patient makes the patient unsuitable for graft repair. Aneurysm is not the only condition for which enzymatic degradation of structural proteins is a hallmark. Other conditions in which structural protein degradation appears to play a key role include Marfan syndrome, supravalvular aortic stenosis. For those afflicted, such conditions lead to, at the very least, a lowered quality of life and often, premature death.

A stent is a metal or plastic tube inserted into the lumen of an anatomic vessel or duct to keep the passageway open. Vascular stents are commonly placed as part of peripheral artery angioplasty. Common sites treated with peripheral artery stents include the carotid, iliac, and femoral arteries. Because of the external compression and mechanical forces subjected to these locations, flexible stent materials such as nitinol are used in a majority of peripheral stent placements. A stent graft or covered stent is type of vascular stent with a fabric coating that creates a contained tube but is expandable like a bare metal stent. Covered stents are used in endovascular surgical procedures such as endovascular aneurysm repair.

A drug-eluting stent is a peripheral or coronary stent placed into narrowed, diseased peripheral or coronary arteries that slowly releases a therapeutic drug. Commonly used drugs block cell proliferation. This prevents fibrosis that, together with clots (thrombi), could otherwise block the stented artery, a process called restenosis. The stent is usually placed within the peripheral or coronary artery by an interventional cardiologist or interventional radiologist during an angioplasty procedure.

A cerebral aneurysm (also called an intracranial aneurysm, neuro aneurysm, or brain aneurysm) is a bulging, weakened area in the wall of an artery in the brain, resulting in an abnormal widening, ballooning, or bleb. Because there is a weakened spot in the aneurysm wall, there is a risk for rupture (bursting) of the aneurysm.

A cerebral aneurysm more frequently occurs in an artery located in the front part of the brain that supplies oxygen-rich blood to the brain tissue. Arteries anywhere in the brain can develop aneurysms. A normal artery wall is made up of three layers. The aneurysm wall is thin and weak because of an abnormal loss or absence of the muscular layer of the artery wall, leaving only two layers.

The most common type of cerebral aneurysm is called a saccular, or berry, aneurysm, occurring in 90 percent of cerebral aneurysms. This type of aneurysm looks like a “berry” with a narrow stem. More than one aneurysm may be present.

Two other types of cerebral aneurysms are fusiform and dissecting aneurysms. A fusiform aneurysm bulges out on all sides (circumferentially), forming a dilated artery. Fusiform aneurysms are often associated with atherosclerosis.

A dissecting aneurysm results from a tear along the length of the artery in the inner layer of the artery wall, causing blood to leak in between the layers of the wall. This may cause a ballooning out on one side of the artery wall, or it may block off or obstruct blood flow through the artery. Dissecting aneurysms usually occur from traumatic injury, but they can also happen spontaneously. The shape and location of the aneurysm may determine which treatment is recommended.

Most cerebral aneurysms (90 percent) present without any symptoms and are small in size (less than 10 millimeters, or less than four-tenths of an inch, in diameter). Smaller aneurysms may have a lower risk of rupture.

Although a cerebral aneurysm may be present without symptoms, the most common initial symptom of a cerebral saccular aneurysm is a sudden headache from a subarachnoid hemorrhage (SAH). SAH is bleeding into the subarachnoid space (the space between the brain and the membranes that cover the brain) and not into the brain tissue. Minor subarachnoid hemorrhage most frequently occurs following head trauma. Major subarachnoid hemorrhage is most commonly from a ruptured cerebral saccular aneurysm (80 percent). A sudden headache associated with SAH is a medical emergency.

Increased risk for aneurysm rupture is associated with aneurysms that are over 10 millimeters (less than four-tenths of an inch) in diameter, a location (circulation in the back portion of the brain), and/or previous rupture of another aneurysm. A significant risk of death is associated with the rupture of a cerebral aneurysm.

Hemorrhagic strokes occur when a blood vessel that supplies the brain ruptures and bleeds. When an artery bleeds into the brain, brain cells and tissues do not receive oxygen and nutrients. In addition, pressure builds up in surrounding tissues, and irritation and swelling occurs. About 20 percent of strokes are caused by hemorrhagic bleeding.

Increased risk of rupture is associated with aneurysms that are greater than 10 millimeters (less than four-tenths of an inch) in diameter, a particular location (circulation in the back portion of the brain), and/or previous rupture of another aneurysm. A significant risk of death is associated with the rupture of a cerebral aneurysm.

Currently, the cause of cerebral aneurysms is not clearly understood. Brain aneurysms are associated with several factors, including smoking, hypertension, and family history (genetic). The ultimate cause of a brain aneurysm is an abnormal degenerative (breaking down) change (weakening) in the wall of an artery, and the effects of pressure from the pulsations of blood being pumped forward through the arteries in the brain. Certain locations of an aneurysm may create greater pressure on the aneurysm, such as at a bifurcation (where the artery divides into smaller branches).

Inherited risk factors associated with aneurysm formation may include, but are not limited to, the following: alpha-glucosidase deficiency (a complete or partial deficiency of the lysosomal enzyme, alpha-glucosidase. This enzyme is necessary to break down glycogen and to convert it into glucose), alpha 1-antitrypsin deficiency (a hereditary disease that may lead to hepatitis and cirrhosis of the liver or emphysema of the lungs), arteriovenous malformation (AVM) (an abnormal connection between an artery and a vein), coarctation of the aorta (a narrowing of the aorta, which is the main artery coming from the heart), Ehlers-Danlos syndrome (a connective tissue disorder), family history of aneurysms, female gender, fibromuscular dysplasia (an arterial disease, cause unknown, that most often affects the medium and large arteries of young to middle-aged women), hereditary hemorrhagic telangiectasia (a genetic disorder of the blood vessels in which there is a tendency to form blood vessels that lack capillaries between an artery and vein), Klinefelter syndrome (a genetic condition in men in which an extra X sex chromosome is present), Noonan's syndrome (a genetic disorder that causes abnormal development of many parts and systems of the body), polycystic kidney disease (PCKD) (a genetic disorder characterized by the growth of numerous cysts filled with fluid in the kidneys. PCKD is the most common medical disease associated with saccular aneurysms), tuberous sclerosis (a type of neurocutaneous syndrome that can cause tumors to grow inside the brain, spinal cord, organs, skin, and skeletal bones)

Acquired risk factors associated with aneurysm formation may include, but are not limited to, the following: advancing age, alcohol consumption (especially binge drinking), atherosclerosis (a buildup of plaque made up of deposits of fatty substances, cholesterol, cellular waste products, calcium, and fibrin in the inner lining of an artery), cigarette smoking, use of illicit drugs, such as cocaine or amphetamine, hypertension (high blood pressure), trauma (injury) to the head, or infection.

The presence of a cerebral aneurysm may not be known until the time of rupture. However, occasionally there may be symptoms that occur prior to an actual rupture due to a small amount of blood that may leak, called “sentinel hemorrhage” into the brain. Some aneurysms are symptomatic because they press on adjacent structures, such as nerves to the eye. They can cause visual loss or diminished eye movements, even if the aneurysm has not ruptured.

The symptoms of an unruptured cerebral aneurysm include, but are not limited to, the following: headaches (rare, if unruptured), eye pain, vision deficits (problems with seeing), or eye movement deficits.

The first evidence of a cerebral aneurysm is most frequently a subarachnoid hemorrhage (SAH), due to rupture of the aneurysm. Symptoms that may occur at the time of SAH include, but are not limited to, the following: initial sign (rapid onset of “worst headache of my life”), stiff neck, nausea and vomiting, changes in mental status, such as drowsiness, pain in specific areas, such as the eyes, dilated pupils, loss of consciousness, hypertension (high blood pressure), motor deficits (loss of balance or coordination), photophobia (sensitivity to light), back or leg pain, cranial nerve deficits (problems with certain functions of the eyes, nose, tongue, and/or ears that are controlled by one or more of the 12 cranial nerves), coma and death.

The symptoms of a cerebral aneurysm may resemble other problems or medical conditions. A cerebral aneurysm is often discovered after it has ruptured or by chance during diagnostic examinations, such as computed tomography (CT scan), magnetic resonance imaging (MRI), or angiography that are being done for other reasons. In addition to a complete medical history and physical examination, diagnostic procedures for a cerebral aneurysm may include digital subtraction angiography (DSA). This provides an image of the blood vessels in the brain to detect a problem with vessels and blood flow. The procedure involves inserting a catheter (a small, thin tube) into an artery in the leg and passing it up to the blood vessels in the brain. Contrast dye is injected through the catheter and X-ray images are taken of the blood vessels. Computed tomography scan (CT or CAT scan) is a diagnostic imaging procedure that uses a combination of X-rays and computer technology to produce horizontal, or axial, images (often called slices) of the body. A CT scan shows detailed images of any part of the body, including the bones, muscles, fat, and organs. CT scans are more detailed than general X-rays, and may be used to detect abnormalities and help identify the location or type of stroke. A CT angiogram (CTA) can also be obtained on a CT scan to look at the vessels. Magnetic resonance imaging (MRI) is a diagnostic procedure that uses a combination of large magnets, radiofrequencies, and a computer to produce detailed images of organs and structures within the body. An MRI uses magnetic fields to detect small changes in brain tissue that help to locate and diagnose a stroke. Magnetic resonance angiography (MRA) is a noninvasive diagnostic procedure that uses a combination of magnetic resonance technology (MRI) and intravenous (IV) contrast dye to visualize blood vessels. Contrast dye causes blood vessels to appear opaque on the MRI image, allowing the doctor to visualize the blood vessels being evaluated.

Specific treatment for a cerebral aneurysm will be determined by a doctor based on: age, overall health, and medical history, extent of the condition, signs and symptoms, tolerance for specific medications, procedures, or therapies, expectations for the course of the condition, and patient opinion or preference.

Depending on situation, the doctor will make recommendations for the appropriate intervention. Whichever intervention is chosen, the main goal is to decrease the risk of subarachnoid hemorrhage, either initially or from a repeated episode of bleeding.

Many factors are considered when making treatment decisions for a cerebral aneurysm. The size and location of the aneurysm, the presence or absence of symptoms, the patient's age and medical condition, and the presence or absence of other risk factors for aneurysm rupture are considered. In some cases, the aneurysm may not be treated and the patient will be closely followed by a doctor. In other cases, surgical treatment may be indicated.

There are two primary surgical treatments for a cerebral aneurysm. First is an open craniotomy (surgical clipping). This procedure involves the surgical removal of part of the skull. The doctor exposes the aneurysm and places a metal clip across the neck of the aneurysm to prevent blood flow into the aneurysm sac. Once the clipping is completed, the skull is secured back together. Second is an endovascular coiling or coil embolization. Endovascular coiling is a minimally invasive technique, which means an incision in the skull is not required to treat the cerebral aneurysm. Rather, a catheter is advanced from a blood vessel in the groin up into the blood vessels in the brain. Fluoroscopy (live X-ray) will be used to assist in advancing the catheter into the head and into the aneurysm. Once the catheter is in place, very tiny platinum coils are advanced through the catheter into the aneurysm. These tiny, soft, platinum coils, which are visible on X-ray, conform to the shape of the aneurysm. The coiled aneurysm becomes clotted off (embolization), preventing rupture. This procedure is performed either under general or local anesthesia.

Phenolic compounds are a diverse group of materials that have been recognized for use in a wide variety of applications. For instance, they naturally occur in many plants, and are often a component of the human diet. Phenolic compounds have been examined in depth for efficacy as free radical scavengers and neutralizers, for instance in topical skin applications and in food supplements. Phenolic compounds are also believed to prevent cross-linking of cell membranes found in certain inflammatory conditions and are believed to affect the expressions of specific genes due to their modulation of free radicals and other oxidative species (see, for example, U.S. patent application Ser. No. 6,437,004 to Perricone).

### SUMMARY

### Endovascular Grafts

What is needed in the art are treatment protocols and compositions for stabilization of the organs and tissues affected in connection with endovascular graft procedures, e.g., the implantation of endovascular grafts or the treatment of leaking endovascular grafts. For example, PGG and/or LeGoo® can be delivered behind an existing stent graft using a microcatheter or a weeping balloon.

In a first aspect (i.e., independently combinable with any of the other aspects or embodiments identified herein), a device is provided for treatment of an aortic aneurysm, comprising: an endovascular graft; and pentagalloyl glucose (PGG).

In an embodiment of the first aspect (i.e., independently combinable with any of the other aspects or embodiments identified herein), the PGG has a purity greater than or equal to 99%.

In an embodiment of the first aspect (i.e., independently combinable with any of the other aspects or embodiments identified herein), at least a portion of the endovascular graft is coated with the PGG.

In an embodiment of the first aspect (i.e., independently combinable with any of the other aspects or embodiments identified herein), at least a portion of a component of the endovascular graft is impregnated with the PGG.

In an embodiment of the first aspect (i.e., independently combinable with any of the other aspects or embodiments identified herein), the PGG is in admixture with a poloxamer gel.

In a second aspect (i.e., independently combinable with any of the other aspects or embodiments identified herein), a device is provided for treating an aorta, comprising: a shaft; and a first balloon attached to a first end of the shaft and comprising a plurality of pores for delivering a therapeutic agent to a target site.

In an embodiment of the second aspect (i.e., independently combinable with any of the other aspects or embodiments identified herein), the device further comprises a second balloon positioned within the first balloon for expanding the first balloon, the second balloon expandable with saline.

In an embodiment of the second aspect (i.e., independently combinable with any of the other aspects or embodiments identified herein), a leaking endovascular graft is present in the target site.

In an embodiment of the second aspect (i.e., independently combinable with any of the other aspects or embodiments identified herein), the device further comprises an endovascular graft supported by the first balloon, wherein the target site contains an aortic aneurysm.

In a third aspect (i.e., independently combinable with any of the other aspects or embodiments identified herein), a kit is provided for treating an aortic aneurysm, comprising: the device of any one of the first or second aspects or embodiments thereof; pentagalloyl glucose (PGG); and a hydrolyzer.

In an embodiment of the third aspect (i.e., independently combinable with any of the other aspects or embodiments identified herein), the PGG has a purity greater than or equal to 99%.

In an embodiment of the third aspect (i.e., independently combinable with any of the other aspects or embodiments identified herein), the hydrolyzer is ethanol, dimethyl sulfoxide (DMSO) or contrast media.

In an embodiment of the third aspect (i.e., independently combinable with any of the other aspects or embodiments identified herein), the PGG is in admixture with a biocompatible poloxamer gel having reverse thermosensitive properties.

In an embodiment of the third aspect (i.e., independently combinable with any of the other aspects or embodiments identified herein), the kit further comprises a saline solution.

In a fourth aspect (i.e., independently combinable with any of the other aspects or embodiments identified herein), a catheter is provided, comprising: an elongate body configured to be introduced into a target site in an artery, the elongate body having a proximal end, a distal end, and a main shaft having a lumen extending therethrough; and a first inflatable balloon coupled to the distal end of the elongate body, the first inflatable balloon having an interior volume in fluid communication with a first inflation lumen, wherein the first inflatable balloon circumferentially surrounds the elongate body, wherein the first inflatable balloon comprises a plurality of pores disposed on a surface of the first inflatable balloon configured to place the interior volume of the first inflatable balloon in fluid communication with the target site.

In an embodiment of the fourth aspect (i.e., independently combinable with any of the other aspects or embodiments identified herein), the first inflatable balloon is generally toroidal forming an annular interior volume that surrounds the elongate body.

In an embodiment of the fourth aspect (i.e., independently combinable with any of the other aspects or embodiments identified herein), the pores are disposed on a central portion of the first inflatable balloon.

In an embodiment of the fourth aspect (i.e., independently combinable with any of the other aspects or embodiments identified herein), the pores are disposed on a distal portion of the first inflatable balloon.

In an embodiment of the fourth aspect (i.e., independently combinable with any of the other aspects or embodiments identified herein), the pores are not disposed on a proximal portion of the first inflatable balloon.

In an embodiment of the fourth aspect (i.e., independently combinable with any of the other aspects or embodiments identified herein), the pores are not disposed on any portion of the first inflatable balloon proximal to a maximum expanded diameter of the balloon in an inflated configuration.

In an embodiment of the fourth aspect (i.e., independently combinable with any of the other aspects or embodiments identified herein), the maximum expanded diameter of the first inflatable balloon is greater than the maximum expanded diameter of the first inflatable balloon.

In an embodiment of the fourth aspect (i.e., independently combinable with any of the other aspects or embodiments identified herein), the catheter further comprises a second inflatable balloon disposed within the interior volume of the first inflatable balloon, the second inflatable balloon having an interior volume in fluid communication with a second inflation lumen.

In an embodiment of the fourth aspect (i.e., independently combinable with any of the other aspects or embodiments identified herein), expansion of the second inflatable balloon is configured to at least partially expand the first inflatable balloon.

In an embodiment of the fourth aspect (i.e., independently combinable with any of the other aspects or embodiments identified herein), expansion of the second inflatable balloon is configured to facilitate expulsion of at least a partial volume of inflation fluid disposed within the interior volume of the first inflatable balloon through the pores into the environment of the target site.

In an embodiment of the fourth aspect (i.e., independently combinable with any of the other aspects or embodiments identified herein), a leaking endovascular graft is present in the target site.

In an embodiment of the fourth aspect (i.e., independently combinable with any of the other aspects or embodiments identified herein), the catheter further comprises an endovascular graft supported by the first balloon, wherein the target site contains an aortic aneurysm.

In a fifth aspect (i.e., independently combinable with any of the other aspects or embodiments identified herein), a kit is provided, comprising: the catheter of the fourth aspect or any of its embodiments; pentagalloyl glucose (PGG); and a hydrolyzer.

In an embodiment of the fifth aspect (i.e., independently combinable with any of the other aspects or embodiments identified herein), the PGG has a purity greater than or equal to 99%.

In an embodiment of the fifth aspect (i.e., independently combinable with any of the other aspects or embodiments identified herein), the hydrolyzer is ethanol, dimethyl sulfoxide (DMSO) or contrast media.

In an embodiment of the fifth aspect (i.e., independently combinable with any of the other aspects or embodiments identified herein), the PGG is in admixture with a biocompatible poloxamer gel having reverse thermosensitive properties.

In an embodiment of the fifth aspect (i.e., independently combinable with any of the other aspects or embodiments identified herein), the kit further comprises a saline solution.

In a sixth aspect (i.e., independently combinable with any of the other aspects or embodiments identified herein), a method is provided for treating a leaking endovascular graft of a patient, comprising: positioning a first balloon in an artery in a region of the leaking endovascular graft; expanding the first balloon such that it presses against surfaces of the artery or the leaking endovascular graft in contact with a surface of the first balloon; and delivering a therapeutic agent to tissue in the region of the leaking endovascular graft through pores in the first balloon.

In a seventh aspect (i.e., independently combinable with any of the other aspects or embodiments identified herein), a method is provided for treating an aortic aneurysm in a patient, comprising: positioning a first balloon in a region of an aortic aneurysm, the first balloon supporting an endovascular graft; expanding the first balloon such that the endovascular graft is implanted in the artery; and delivering a therapeutic agent to tissue of the region through pores in the first balloon.

In an embodiment of the sixth or seventh aspect (i.e., independently combinable with any of the other aspects or embodiments identified herein), expanding the first balloon comprises introducing an inflation fluid into an interior volume of the first balloon.

In an embodiment of the sixth or seventh aspect (i.e., independently combinable with any of the other aspects or embodiments identified herein), delivering the therapeutic agent comprises introducing a solution comprising the therapeutic agent into an interior volume of the first balloon, the introduction of the solution being configured to expand and/or maintain an expanded state of the first balloon.

In an embodiment of the sixth or seventh aspect (i.e., independently combinable with any of the other aspects or embodiments identified herein), expanding the first balloon comprises maintaining a pressure within an interior volume of the second balloon greater than a diastolic blood pressure of the patient and less than a systolic blood pressure of the patient.

In an embodiment of the sixth or seventh aspect (i.e., independently combinable with any of the other aspects or embodiments identified herein), expanding the first balloon and delivering the therapeutic agent through the pores comprises introducing a solution into an interior volume of the first balloon, and wherein the solution is introduced at a first volumetric flow rate to expand the first balloon and the solution is introduced at a second volumetric flow rate to deliver the therapeutic agent through the pores, the first volumetric flow rate being greater than or equal to the second volumetric flow rate.

In an embodiment of the sixth or seventh aspect (i.e., independently combinable with any of the other aspects or embodiments identified herein), the first volumetric flow rate is greater than the second volumetric flow rate.

In an embodiment of the sixth or seventh aspect (i.e., independently combinable with any of the other aspects or embodiments identified herein), blood flow is occluded by the first balloon no longer than approximately 3 minutes.

In an embodiment of the sixth or seventh aspect (i.e., independently combinable with any of the other aspects or embodiments identified herein), at least 1 mL of solution comprising the therapeutic agent is delivered while downstream blood flow and retrograde blood flow is occluded.

In an embodiment of the sixth or seventh aspect (i.e., independently combinable with any of the other aspects or embodiments identified herein), expanding the first balloon comprises inflating a second balloon disposed within an interior volume of the first balloon.

In an embodiment of the sixth or seventh aspect (i.e., independently combinable with any of the other aspects or embodiments identified herein), delivering the therapeutic agent comprises inflating a second balloon disposed within an interior volume of the first balloon to force a volume of solution comprising the therapeutic agent within the interior volume of the first balloon through the pores.

In an embodiment of the sixth or seventh aspect (i.e., independently combinable with any of the other aspects or embodiments identified herein), the therapeutic agent comprises pentagalloyl glucose (PGG).

In an embodiment of the sixth or seventh aspect (i.e., independently combinable with any of the other aspects or embodiments identified herein), the PGG is at least 99.9% pure.

In an embodiment of the sixth or seventh aspect (i.e., independently combinable with any of the other aspects or embodiments identified herein), the therapeutic agent is substantially free of gallic acid or methyl gallate.

In an embodiment of the sixth or seventh aspect (i.e., independently combinable with any of the other aspects or embodiments identified herein), the therapeutic agent is in admixture with a biocompatible poloxamer gel having reverse thermosensitive properties.

In an eighth aspect (i.e., independently combinable with any of the other aspects or embodiments identified herein), a method is provided for treating a leaking endovascular graft of a patient, comprising: delivering a therapeutic agent to tissue in a region of the leaking endovascular graft, wherein the therapeutic agent comprises at least one of pentagalloyl glucose (PGG) and a biocompatible poloxamer gel having reverse thermosensitive properties.

In an embodiment of the eighth aspect (i.e., independently combinable with any of the other aspects or embodiments identified herein), the therapeutic agent comprises PGG, and wherein the PGG is at least 99.9% pure.

In an embodiment of the eighth aspect (i.e., independently combinable with any of the other aspects or embodiments identified herein), the therapeutic agent is substantially free of gallic acid or methyl gallate.

In an embodiment of the eighth aspect (i.e., independently combinable with any of the other aspects or embodiments identified herein), the therapeutic agent is an admixture of the PGG and the biocompatible poloxamer gel.

In an embodiment of the eighth aspect (i.e., independently combinable with any of the other aspects or embodiments identified herein), the region is situated behind the leaking endovascular graft.

In an embodiment of the eighth aspect (i.e., independently combinable with any of the other aspects or embodiments identified herein), the therapeutic agent is delivered by a microcatheter.

In an embodiment of the eighth aspect (i.e., independently combinable with any of the other aspects or embodiments identified herein), the therapeutic agent is delivered by a weeping balloon. Dissections

What is needed in the art are treatment protocols and compositions for stabilization of the organs and tissues affected by aortic or thoracic dissections, or dissections of other arteries.

In a first aspect (i.e., independently combinable with any of the other aspects or embodiments identified herein), a device is provided for treatment of a dissection of an artery, comprising: an implantable stent or stent graft; and pentagalloyl glucose (PGG).

In an embodiment of the first aspect (i.e., independently combinable with any of the other aspects or embodiments identified herein), the PGG has a purity greater than or equal to 99%.

In an embodiment of the first aspect (i.e., independently combinable with any of the other aspects or embodiments identified herein), at least a portion of the implantable stent or stent graft is coated with the PGG.

In an embodiment of the first aspect (i.e., independently combinable with any of the other aspects or embodiments identified herein), at least a portion of a component of the implantable stent or stent graft is impregnated with the PGG.

In an embodiment of the first aspect (i.e., independently combinable with any of the other aspects or embodiments identified herein), the PGG is in admixture with a poloxamer gel.

In a second aspect (i.e., independently combinable with any of the other aspects or embodiments identified herein), a device is provided for treating a dissection, comprising: a shaft; and a first balloon attached to a first end of the shaft and comprising a plurality of pores for delivering a therapeutic agent to a dissection, an implantation site, or a surgical site.

In an embodiment of the second aspect (i.e., independently combinable with any of the other aspects or embodiments identified herein), the device further comprises an implantable stent or stent graft supported by the first balloon.

In an embodiment of the second aspect (i.e., independently combinable with any of the other aspects or embodiments identified herein), the device further comprises a second balloon positioned within the first balloon for expanding the first balloon, the second balloon expandable with saline.

In a third embodiment, a kit is provided for treating a dissection of an artery, comprising: the device of any one of the first or second aspects or their embodiments; pentagalloyl glucose (PGG); and a hydrolyzer.

In an embodiment of the third aspect (i.e., independently combinable with any of the other aspects or embodiments identified herein), the PGG has a purity greater than or equal to 99%.

In an embodiment of the third aspect (i.e., independently combinable with any of the other aspects or embodiments identified herein), the hydrolyzer is ethanol, dimethyl sulfoxide (DMSO) or contrast media.

In an embodiment of the third aspect (i.e., independently combinable with any of the other aspects or embodiments identified herein), the PGG is in admixture with a biocompatible poloxamer gel having reverse thermosensitive properties.

In an embodiment of the third aspect (i.e., independently combinable with any of the other aspects or embodiments identified herein), the kit further comprises a saline solution.

In a fourth aspect (i.e., independently combinable with any of the other aspects or embodiments identified herein), a catheter is provided for treating a dissection of an artery, the catheter comprising: an elongate body configured to be introduced into a surgical site or implantation site of an artery having a dissection, the elongate body having a proximal end, a distal end, and a main shaft having a lumen extending therethrough; and a first inflatable balloon coupled to the distal end of the elongate body, the first inflatable balloon having an interior volume in fluid communication with a first inflation lumen, wherein the first inflatable balloon circumferentially surrounds the elongate body, wherein the first inflatable balloon comprises a plurality of pores disposed on a surface of the first inflatable balloon configured to place the interior volume of the first inflatable balloon in fluid communication with the dissection, the artery, the implantation site, or the surgical site.

In an embodiment of the fourth aspect (i.e., independently combinable with any of the other aspects or embodiments identified herein), the first inflatable balloon is further configured to support an implantable stent or stent graft.

In an embodiment of the fourth aspect (i.e., independently combinable with any of the other aspects or embodiments identified herein), the first inflatable balloon is generally toroidal forming an annular interior volume that surrounds the elongate body.

In an embodiment of the fourth aspect (i.e., independently combinable with any of the other aspects or embodiments identified herein), the pores are disposed on a central portion of the first inflatable balloon.

In an embodiment of the fourth aspect (i.e., independently combinable with any of the other aspects or embodiments identified herein), the pores are disposed on a distal portion of the first inflatable balloon.

In an embodiment of the fourth aspect (i.e., independently combinable with any of the other aspects or embodiments identified herein), the pores are not disposed on a proximal portion of the first inflatable balloon.

In an embodiment of the fourth aspect (i.e., independently combinable with any of the other aspects or embodiments identified herein), the pores are not disposed on any portion of the first inflatable balloon proximal to a maximum expanded diameter of the balloon in an inflated configuration.

In an embodiment of the fourth aspect (i.e., independently combinable with any of the other aspects or embodiments identified herein), the maximum expanded diameter of the first inflatable balloon is greater than the maximum expanded diameter of the first inflatable balloon.

In an embodiment of the fourth aspect (i.e., independently combinable with any of the other aspects or embodiments identified herein), the catheter further comprises a second inflatable balloon disposed within the interior volume of the first inflatable balloon, the second inflatable balloon having an interior volume in fluid communication with a second inflation lumen.

In an embodiment of the fourth aspect (i.e., independently combinable with any of the other aspects or embodiments identified herein), expansion of the second inflatable balloon is configured to at least partially expand the first inflatable balloon.

In an embodiment of the fourth aspect (i.e., independently combinable with any of the other aspects or embodiments identified herein), expansion of the second inflatable balloon is configured to facilitate expulsion of at least a partial volume of inflation fluid disposed within the interior volume of the first inflatable balloon through the pores into the environment of the dissection, the implantation site, or the surgical site.

In a fifth aspect (i.e., independently combinable with any of the other aspects or embodiments identified herein), a kit is provided for treating a dissection, comprising: the catheter of the fifth aspect or any of its embodiments; pentagalloyl glucose (PGG); and a hydrolyzer.

In an embodiment of the fifth aspect (i.e., independently combinable with any of the other aspects or embodiments identified herein), the PGG has a purity greater than or equal to 99%.

In an embodiment of the fifth aspect (i.e., independently combinable with any of the other aspects or embodiments identified herein), the hydrolyzer is ethanol, dimethyl sulfoxide (DMSO) or contrast media.

In an embodiment of the fifth aspect (i.e., independently combinable with any of the other aspects or embodiments identified herein), the PGG is in admixture with a biocompatible poloxamer gel having reverse thermosensitive properties.

In an embodiment of the fifth aspect (i.e., independently combinable with any of the other aspects or embodiments identified herein), the kit further comprises a saline solution.

In a sixth aspect (i.e., independently combinable with any of the other aspects or embodiments identified herein), a method is provided for treating a dissection of an artery of a patient, comprising: positioning a first balloon in an artery in a region of the dissection; expanding the first balloon such that it presses against surfaces of the artery in contact with a surface of the first balloon; and delivering a therapeutic agent to the dissection through pores in the first balloon.

In a seventh aspect (i.e., independently combinable with any of the other aspects or embodiments identified herein), a method is provided for treating a dissection of an artery of a patient, comprising: positioning a first balloon in a region of the dissection, the first balloon supporting an implantable stent or stent graft; expanding the first balloon such that the implantable stent or stent graft is implanted in the artery; and delivering a therapeutic agent to the implantation site through pores in the first balloon.

In an embodiment of the sixth or seventh aspect (i.e., independently combinable with any of the other aspects or embodiments identified herein), expanding the first balloon comprises introducing an inflation fluid into an interior volume of the first balloon.

In an embodiment of the sixth or seventh aspect (i.e., independently combinable with any of the other aspects or embodiments identified herein), delivering the therapeutic agent comprises introducing a solution comprising the therapeutic agent into an interior volume of the first balloon, the introduction of the solution being configured to expand and/or maintain an expanded state of the first balloon.

In an embodiment of the sixth or seventh aspect (i.e., independently combinable with any of the other aspects or embodiments identified herein), expanding the first balloon comprises maintaining a pressure within an interior volume of the second balloon greater than a diastolic blood pressure of the patient and less than a systolic blood pressure of the patient.

In an embodiment of the sixth or seventh aspect (i.e., independently combinable with any of the other aspects or embodiments identified herein), expanding the first balloon and delivering the therapeutic agent through the pores comprises introducing a solution into an interior volume of the first balloon, and wherein the solution is introduced at a first volumetric flow rate to expand the first balloon and the solution is introduced at a second volumetric flow rate to deliver the therapeutic agent through the pores, the first volumetric flow rate being greater than or equal to the second volumetric flow rate.

In an embodiment of the sixth or seventh aspect (i.e., independently combinable with any of the other aspects or embodiments identified herein), the first volumetric flow rate is greater than the second volumetric flow rate.

In an embodiment of the sixth or seventh aspect (i.e., independently combinable with any of the other aspects or embodiments identified herein), blood flow is occluded by the first balloon no longer than approximately 3 minutes.

In an embodiment of the sixth or seventh aspect (i.e., independently combinable with any of the other aspects or embodiments identified herein), at least 1 mL of solution comprising the therapeutic agent is delivered while downstream blood flow and retrograde blood flow is occluded.

In an embodiment of the sixth or seventh aspect (i.e., independently combinable with any of the other aspects or embodiments identified herein), expanding the first balloon comprises inflating a second balloon disposed within an interior volume of the first balloon.

In an embodiment of the sixth or seventh aspect (i.e., independently combinable with any of the other aspects or embodiments identified herein), delivering the therapeutic agent comprises inflating a second balloon disposed within an interior volume of the first balloon to force a volume of solution comprising the therapeutic agent within the interior volume of the first balloon through the pores.

In an embodiment of the sixth or seventh aspect (i.e., independently combinable with any of the other aspects or embodiments identified herein), the therapeutic agent comprises pentagalloyl glucose (PGG).

In an embodiment of the sixth or seventh aspect (i.e., independently combinable with any of the other aspects or embodiments identified herein), the PGG is at least 99.9% pure.

In an embodiment of the sixth or seventh aspect (i.e., independently combinable with any of the other aspects or embodiments identified herein), the therapeutic agent is substantially free of gallic acid or methyl gallate.

In an eighth aspect (i.e., independently combinable with any of the other aspects or embodiments identified herein), a method is provided for treating a dissection of an artery of a patient, comprising: delivering a therapeutic agent to tissue of the dissection or tissue in a region of the dissection, wherein the therapeutic agent comprises pentagalloyl glucose (PGG).

In an embodiment of the eighth aspect (i.e., independently combinable with any of the other aspects or embodiments identified herein), the PGG is at least 99.9% pure.

In an embodiment of the eighth aspect (i.e., independently combinable with any of the other aspects or embodiments identified herein), the therapeutic agent is substantially free of gallic acid or methyl gallate.

In an embodiment of the eighth aspect (i.e., independently combinable with any of the other aspects or embodiments identified herein), the artery is an aorta.

In an embodiment of the eighth aspect (i.e., independently combinable with any of the other aspects or embodiments identified herein), the artery is a thoracic artery.

In an embodiment of the eighth aspect (i.e., independently combinable with any of the other aspects or embodiments identified herein), the therapeutic agent is PGG in admixture with a biocompatible poloxamer gel having reverse thermosensitive properties. Peripheral Aneurysms

What is needed in the art are treatment protocols and compositions for stabilization of the organs and tissues affected by degenerative conditions such as peripheral aneurysm. In particular, treatment protocols utilizing phenolic compounds could provide a safe, less invasive route for the stabilization of the structural architecture in order to temper growth and/or development of such conditions.

In a first aspect (i.e., independently combinable with any of the other aspects or embodiments identified herein), a device is provided for treating a peripheral aneurysm, comprising: a stent graft; and pentagalloyl glucose (PGG).

In an embodiment of the first aspect (i.e., independently combinable with any of the other aspects or embodiments identified herein), the PGG has a purity greater than or equal to 99%.

In an embodiment of the first aspect (i.e., independently combinable with any of the other aspects or embodiments identified herein), at least a portion of the stent graft is coated with the PGG.

In an embodiment of the first aspect (i.e., independently combinable with any of the other aspects or embodiments identified herein), at least a portion of stent graft is impregnated with the PGG.

In an embodiment of the first aspect (i.e., independently combinable with any of the other aspects or embodiments identified herein), the device further comprises a poloxamer gel exhibiting a reverse thermosensitive property.

In an embodiment of the first aspect (i.e., independently combinable with any of the other aspects or embodiments identified herein), the poloxamer gel is configured to occlude a blood vessel in a region of the peripheral aneurysm.

In an embodiment of the first aspect (i.e., independently combinable with any of the other aspects or embodiments identified herein), at least a portion of the stent graft is coated with the poloxamer gel.

In an embodiment of the first aspect (i.e., independently combinable with any of the other aspects or embodiments identified herein), at least a portion of stent graft is impregnated with the poloxamer gel.

In an embodiment of the first aspect (i.e., independently combinable with any of the other aspects or embodiments identified herein), the PGG is admixed with the poloxamer gel.

In an embodiment of the first aspect (i.e., independently combinable with any of the other aspects or embodiments identified herein), the device further comprises a shaft; a first balloon attached to a first end of the shaft; and a second balloon attached to a second end of the shaft, the second balloon comprising a plurality of pores for delivering a therapeutic agent to the peripheral aneurysm.

In an embodiment of the first aspect (i.e., independently combinable with any of the other aspects or embodiments identified herein), the first balloon is positioned near a distal end of the shaft for anchoring the device and stopping downstream blood flow, and wherein the second balloon is positioned near a proximal end of the shaft for stopping retrograde blood flow and/or for displacing blood from an aneurysmal sac.

In an embodiment of the first aspect (i.e., independently combinable with any of the other aspects or embodiments identified herein), the second balloon is positioned near a distal end of the shaft for anchoring the device and stopping downstream blood flow, and wherein the first balloon is positioned near a proximal end of the shaft for stopping retrograde blood flow.

In an embodiment of the first aspect (i.e., independently combinable with any of the other aspects or embodiments identified herein), the device further comprises a third balloon positioned within the second balloon for expanding the second balloon, the third balloon expandable with saline.

In a second aspect (i.e., independently combinable with any of the other aspects or embodiments identified herein), a kit is provided for treating a peripheral aneurysm, comprising: the device of any one of claims 1 to 8; PGG having a purity greater than or equal to 99%; and a hydrolyzer.

In an embodiment of the second aspect (i.e., independently combinable with any of the other aspects or embodiments identified herein), the hydrolyzer is ethanol.

In an embodiment of the second aspect (i.e., independently combinable with any of the other aspects or embodiments identified herein), the hydrolyzer is dimethyl sulfoxide (DMSO) or contrast media.

In an embodiment of the second aspect (i.e., independently combinable with any of the other aspects or embodiments identified herein), the kit further comprises a saline solution.

In a third aspect (i.e., independently combinable with any of the other aspects or embodiments identified herein), a catheter is provided for treating a peripheral aneurysm, the catheter comprising: an elongate body configured to be introduced into a blood vessel, the elongate body having a proximal end, a distal end, and a main shaft having a lumen extending therethrough; a first inflatable balloon coupled to the distal end of the elongate body, the first inflatable balloon having an interior volume in fluid communication with a first inflation lumen; and a second inflatable balloon coupled to the elongate body proximally to the first inflatable balloon, the second inflatable balloon having an interior volume in fluid communication with a second inflation lumen, wherein the second inflatable balloon circumferentially surrounds the elongate body, wherein the second inflatable balloon comprises a plurality of pores disposed on a surface of the second inflatable balloon configured to place the interior volume of the second inflatable balloon in fluid communication with an intravascular environment of the blood vessel, and wherein either the first inflatable balloon or the second inflatable balloon is configured to support a stent graft for placement in the peripheral aneurysm.

In an embodiment of the third aspect (i.e., independently combinable with any of the other aspects or embodiments identified herein), the main shaft extends through the second inflatable balloon and the distal end of the main shaft forms the distal end of the elongate body.

In an embodiment of the third aspect (i.e., independently combinable with any of the other aspects or embodiments identified herein), the first inflation lumen and the second inflation lumen are formed within the main shaft.

In an embodiment of the third aspect (i.e., independently combinable with any of the other aspects or embodiments identified herein), the elongate body further comprises a second shaft having a lumen extending therethrough, the second shaft being disposed within the lumen of the main shaft, the first inflatable balloon being coupled to a distal end of the second shaft and the second inflatable balloon being coupled to a distal end of the main shaft.

In an embodiment of the third aspect (i.e., independently combinable with any of the other aspects or embodiments identified herein), the lumen of the main shaft is the second inflation lumen.

In an embodiment of the third aspect (i.e., independently combinable with any of the other aspects or embodiments identified herein), the lumen of the second shaft is the first inflation lumen.

In an embodiment of the third aspect (i.e., independently combinable with any of the other aspects or embodiments identified herein), the elongate body extends through the interior volume of the second inflatable balloon.

In an embodiment of the third aspect (i.e., independently combinable with any of the other aspects or embodiments identified herein), the second inflatable balloon is generally toroidal forming an annular interior volume that surrounds the elongate body.

In an embodiment of the third aspect (i.e., independently combinable with any of the other aspects or embodiments identified herein), the elongate body comprises an intermediate shaft segment positioned between a proximal end of the first inflatable balloon and a distal end of the second inflatable balloon.

In an embodiment of the third aspect (i.e., independently combinable with any of the other aspects or embodiments identified herein), the intermediate shaft segment comprises the main shaft.

In an embodiment of the third aspect (i.e., independently combinable with any of the other aspects or embodiments identified herein), the intermediate shaft segment comprises the second shaft.

In an embodiment of the third aspect (i.e., independently combinable with any of the other aspects or embodiments identified herein), a separation distance between the first inflatable balloon and the second inflatable balloon is fixed.

In an embodiment of the third aspect (i.e., independently combinable with any of the other aspects or embodiments identified herein), a separation distance between the first inflatable balloon and the second inflatable balloon is adjustable.

In an embodiment of the third aspect (i.e., independently combinable with any of the other aspects or embodiments identified herein), the catheter further comprises a lumen configured to be placed in fluid communication with a volume of the intravascular environment between the first inflatable balloon and the second inflatable balloon.

In an embodiment of the third aspect (i.e., independently combinable with any of the other aspects or embodiments identified herein), the pores are disposed on a central portion of the second inflatable balloon.

In an embodiment of the third aspect (i.e., independently combinable with any of the other aspects or embodiments identified herein), the pores are disposed on a distal portion of the second inflatable balloon.

In an embodiment of the third aspect (i.e., independently combinable with any of the other aspects or embodiments identified herein), the pores are not disposed on a proximal portion of the second inflatable balloon.

In an embodiment of the third aspect (i.e., independently combinable with any of the other aspects or embodiments identified herein), the pores are not disposed on any portion of the second inflatable balloon proximal to a maximum expanded diameter of the balloon in an inflated configuration.

In an embodiment of the third aspect (i.e., independently combinable with any of the other aspects or embodiments identified herein), the maximum expanded diameter of the second inflatable balloon is greater than the maximum expanded diameter of the first inflatable balloon.

In an embodiment of the third aspect (i.e., independently combinable with any of the other aspects or embodiments identified herein), the length of the expanded second inflatable balloon is greater than the length of the expanded first inflatable balloon.

In an embodiment of the third aspect (i.e., independently combinable with any of the other aspects or embodiments identified herein), the catheter further comprises a third inflatable balloon disposed within the interior volume of the second inflatable balloon, the third inflatable balloon having an interior volume in fluid communication with a third inflation lumen.

In an embodiment of the third aspect (i.e., independently combinable with any of the other aspects or embodiments identified herein), expansion of the third inflatable balloon is configured to at least partially expand the second inflatable balloon.

In an embodiment of the third aspect (i.e., independently combinable with any of the other aspects or embodiments identified herein), expansion of the third inflatable balloon is configured to facilitate expulsion of at least a partial volume of inflation fluid disposed within the interior volume of the second inflatable balloon through the pores into the intravascular environment.

In an embodiment of the third aspect (i.e., independently combinable with any of the other aspects or embodiments identified herein), the catheter further comprises a stent graft coated or impregnated with pentagalloyl glucose (PGG).

In an embodiment of the third aspect (i.e., independently combinable with any of the other aspects or embodiments identified herein), the PGG has a purity greater than or equal to 99%.

In an embodiment of the third aspect (i.e., independently combinable with any of the other aspects or embodiments identified herein), at least a portion of the stent graft is coated with the PGG.

In an embodiment of the third aspect (i.e., independently combinable with any of the other aspects or embodiments identified herein), at least a portion of stent graft is impregnated with the PGG.

In a fourth aspect (i.e., independently combinable with any of the other aspects or embodiments identified herein), a method is provided for treating a peripheral aneurysm of a patient, comprising: positioning a first balloon upstream the peripheral aneurysm; positioning a second balloon adjacent the peripheral aneurysm, wherein the second balloon supports a stent graft; inflating the first balloon to occlude downstream blood flow; expanding the second balloon to deploy the stent graft; and delivering a therapeutic agent to the stent graft or the site of the aneurysm through pores in the second balloon.

In an embodiment of the fourth aspect (i.e., independently combinable with any of the other aspects or embodiments identified herein), expanding the second balloon comprises introducing an inflation fluid into an interior volume of the second balloon.

In an embodiment of the fourth aspect (i.e., independently combinable with any of the other aspects or embodiments identified herein), delivering the therapeutic agent comprises introducing a solution comprising the therapeutic agent into an interior volume of the second balloon, the introduction of the solution being configured to expand and/or maintain an expanded state of the second balloon.

In an embodiment of the fourth aspect (i.e., independently combinable with any of the other aspects or embodiments identified herein), inflating the first balloon and expanding the second balloon creates a sealed volume within the blood vessel between the first balloon and the second balloon.

In an embodiment of the fourth aspect (i.e., independently combinable with any of the other aspects or embodiments identified herein), delivering the therapeutic agent comprises introducing the therapeutic agent into the sealed volume.

In an embodiment of the fourth aspect (i.e., independently combinable with any of the other aspects or embodiments identified herein), the therapeutic agent is not delivered into the blood vessel outside of the sealed volume while the sealed volume is established.

In an embodiment of the fourth aspect (i.e., independently combinable with any of the other aspects or embodiments identified herein), inflating the first balloon anchors the first balloon and the second balloon within the blood vessel.

In an embodiment of the fourth aspect (i.e., independently combinable with any of the other aspects or embodiments identified herein), positioning the second balloon adjacent the aneurysm comprises positioning the second balloon across the aneurysm and wherein expanding the second balloon creates a sealed space between the second balloon and the aneurysm.

In an embodiment of the fourth aspect (i.e., independently combinable with any of the other aspects or embodiments identified herein), positioning the second balloon adjacent the aneurysm comprises positioning the second balloon along a downstream edge of the aneurysm and wherein expanding the second balloon creates a sealed volume between the first balloon and the second balloon which encompasses the aneurysm.

In an embodiment of the fourth aspect (i.e., independently combinable with any of the other aspects or embodiments identified herein), positioning the second balloon adjacent the aneurysm comprises positioning the second balloon such that a length of the aneurysm along the blood vessel encompasses an entire length of the second balloon.

In an embodiment of the fourth aspect (i.e., independently combinable with any of the other aspects or embodiments identified herein), inflating the first balloon occurs prior to expanding the second balloon.

In an embodiment of the fourth aspect (i.e., independently combinable with any of the other aspects or embodiments identified herein), expanding the second balloon and/or maintaining the second balloon in an expanded state comprises maintaining a pressure within an interior volume of the second balloon greater than a diastolic blood pressure of the patient and less than a systolic blood pressure of the patient.

In an embodiment of the fourth aspect (i.e., independently combinable with any of the other aspects or embodiments identified herein), expanding the second balloon and delivering the therapeutic agent through the pores comprises introducing a solution into an interior volume of the second balloon, and wherein the solution is introduced at a first volumetric flow rate to expand the second balloon and the solution is introduced at a second volumetric flow rate to deliver the therapeutic agent through the pores, the first volumetric flow rate being greater than or equal to the second volumetric flow rate.

In an embodiment of the fourth aspect (i.e., independently combinable with any of the other aspects or embodiments identified herein), the first volumetric flow rate is greater than the second volumetric flow rate.

In an embodiment of the fourth aspect (i.e., independently combinable with any of the other aspects or embodiments identified herein), blood flow is occluded within the blood vessel for no longer than approximately 3 minutes.

In an embodiment of the fourth aspect (i.e., independently combinable with any of the other aspects or embodiments identified herein), at least 1 mL of solution comprising the therapeutic agent is delivered while downstream blood flow and retrograde blood flow vessel is occluded.

In an embodiment of the fourth aspect (i.e., independently combinable with any of the other aspects or embodiments identified herein), expanding the second balloon comprises inflating a third balloon disposed within an interior volume of the second balloon.

In an embodiment of the fourth aspect (i.e., independently combinable with any of the other aspects or embodiments identified herein), delivering the therapeutic agent comprises inflating a third balloon disposed within an interior volume of the second balloon to force a volume of solution comprising the therapeutic agent within the interior volume of the second balloon through the pores.

In an embodiment of the fourth aspect (i.e., independently combinable with any of the other aspects or embodiments identified herein), the therapeutic agent comprises pentagalloyl glucose (PGG).

In an embodiment of the fourth aspect (i.e., independently combinable with any of the other aspects or embodiments identified herein), the PGG is at least 99.9% pure.

In an embodiment of the fourth aspect (i.e., independently combinable with any of the other aspects or embodiments identified herein), the therapeutic agent is substantially free of gallic acid or methyl gallate.

In a fifth aspect (i.e., independently combinable with any of the other aspects or embodiments identified herein), a method is provided for treating a peripheral aneurysm of a patient, comprising: positioning a stent graft comprising pentagalloyl glucose (PGG) in a blood vessel adjacent to a peripheral aneurysm; and delivering the PGG to the blood vessel or the peripheral aneurysm.

In an embodiment of the fifth aspect (i.e., independently combinable with any of the other aspects or embodiments identified herein), the PGG is at least 99.9% pure.

In an embodiment of the fifth aspect (i.e., independently combinable with any of the other aspects or embodiments identified herein), the therapeutic agent is substantially free of gallic acid or methyl gallate.

In an embodiment of the fifth aspect (i.e., independently combinable with any of the other aspects or embodiments identified herein), the PGG is in admixture with a poloxomer gel.

In an embodiment of the fifth aspect (i.e., independently combinable with any of the other aspects or embodiments identified herein), the method further comprises occluding the blood vessel with a poloxomer gel.

In an embodiment of the fifth aspect (i.e., independently combinable with any of the other aspects or embodiments identified herein), the PGG is in admixture with the poloxomer gel. Neuro Aneurysm

What is needed in the art are treatment protocols and compositions for stabilization of the organs and tissues affected by degenerative conditions such as neuro aneurysms. In particular, treatment protocols utilizing phenolic compounds could provide a safe, less invasive route for the stabilization of the structural architecture in order to temper growth and/or development of such conditions.

In a first aspect (i.e., independently combinable with any of the other aspects or embodiments identified herein), a device is provided for treating a neuro aneurysm, comprising: an implantable coil and pentagalloyl glucose (PGG).

In an embodiment of the first aspect (i.e., independently combinable with any of the other aspects or embodiments identified herein), the PGG has a purity greater than or equal to 99%.

In an embodiment of the first aspect (i.e., independently combinable with any of the other aspects or embodiments identified herein), at least a portion of the implantable coil is coated with the PGG.

In an embodiment of the first aspect (i.e., independently combinable with any of the other aspects or embodiments identified herein), at least a portion of implantable coil is impregnated with the PGG.

In a second aspect (i.e., independently combinable with any of the other aspects or embodiments identified herein), a device is provided for treating a neuro aneurysm, comprising: a shaft configured to deliver an implantable coil to the neuro aneurysm; a first balloon attached to a first end of the shaft; and a second balloon attached to a second end of the shaft, the second balloon comprising a plurality of pores for delivering a therapeutic agent to the aneurysm.

In an embodiment of the second aspect (i.e., independently combinable with any of the other aspects or embodiments identified herein), the first balloon is positioned near a distal end of the shaft for anchoring the device and stopping downstream blood flow, and wherein the second balloon is positioned near a proximal end of the shaft for stopping retrograde blood flow and/or for displacing blood from an aneurysmal sac.

In an embodiment of the second aspect (i.e., independently combinable with any of the other aspects or embodiments identified herein), the second balloon is positioned near a distal end of the shaft for anchoring the device and stopping downstream blood flow, and wherein the first balloon is positioned near a proximal end of the shaft for stopping retrograde blood flow.

In an embodiment of the second aspect (i.e., independently combinable with any of the other aspects or embodiments identified herein), the device further comprises a third balloon positioned within the second balloon for expanding the second balloon, the third balloon expandable with saline.

In a third aspect (i.e., independently combinable with any of the other aspects or embodiments identified herein), a kit for treating a neuro aneurysm is provided, comprising: the device of the second aspect or any of its embodiments; PGG; and a hydrolyzer.

In an embodiment of the third aspect (i.e., independently combinable with any of the other aspects or embodiments identified herein), the hydrolyzer is ethanol.

In an embodiment of the third aspect (i.e., independently combinable with any of the other aspects or embodiments identified herein), the hydrolyzer is dimethyl sulfoxide (DMSO) or contrast media.

In an embodiment of the third aspect (i.e., independently combinable with any of the other aspects or embodiments identified herein), the kit further comprises a saline solution.

In an embodiment of the third aspect (i.e., independently combinable with any of the other aspects or embodiments identified herein), the PGG has a purity greater than or equal to 99%.

In a fourth aspect (i.e., independently combinable with any of the other aspects or embodiments identified herein), a catheter is provided for treating a neuro aneurysm, the catheter comprising: an elongate body configured to be introduced into a blood vessel, the elongate body having a proximal end, a distal end, and a main shaft having a lumen extending therethrough, wherein the lumen is adapted to deliver an implantable coil to the neuro aneurysm.

In an embodiment of the fourth aspect (i.e., independently combinable with any of the other aspects or embodiments identified herein), the catheter further comprises a first inflatable balloon coupled to the distal end of the elongate body, the first inflatable balloon having an interior volume in fluid communication with a first inflation lumen; and a second inflatable balloon coupled to the elongate body proximally to the first inflatable balloon, the second inflatable balloon having an interior volume in fluid communication with a second inflation lumen, wherein the second inflatable balloon circumferentially surrounds the elongate body, and wherein the second inflatable balloon comprises a plurality of pores disposed on a surface of the second inflatable balloon configured to place the interior volume of the second inflatable balloon in fluid communication with an intravascular environment of the blood vessel.

In an embodiment of the fourth aspect (i.e., independently combinable with any of the other aspects or embodiments identified herein), the main shaft extends through the second inflatable balloon and the distal end of the main shaft forms the distal end of the elongate body.

In an embodiment of the fourth aspect (i.e., independently combinable with any of the other aspects or embodiments identified herein), the first inflation lumen and the second inflation lumen are formed within the main shaft.

In an embodiment of the fourth aspect (i.e., independently combinable with any of the other aspects or embodiments identified herein), the elongate body further comprises a second shaft having a lumen extending therethrough, the second shaft being disposed within the lumen of the main shaft, the first inflatable balloon being coupled to a distal end of the second shaft and the second inflatable balloon being coupled to a distal end of the main shaft.

In an embodiment of the fourth aspect (i.e., independently combinable with any of the other aspects or embodiments identified herein), the lumen of the main shaft is the second inflation lumen.

In an embodiment of the fourth aspect (i.e., independently combinable with any of the other aspects or embodiments identified herein), the lumen of the second shaft is the first inflation lumen.

In an embodiment of the fourth aspect (i.e., independently combinable with any of the other aspects or embodiments identified herein), the elongate body extends through the interior volume of the second inflatable balloon.

In an embodiment of the fourth aspect (i.e., independently combinable with any of the other aspects or embodiments identified herein), the second inflatable balloon is generally toroidal forming an annular interior volume that surrounds the elongate body.

In an embodiment of the fourth aspect (i.e., independently combinable with any of the other aspects or embodiments identified herein), the elongate body comprises an intermediate shaft segment positioned between a proximal end of the first inflatable balloon and a distal end of the second inflatable balloon.

In an embodiment of the fourth aspect (i.e., independently combinable with any of the other aspects or embodiments identified herein), the intermediate shaft segment comprises the main shaft.

In an embodiment of the fourth aspect (i.e., independently combinable with any of the other aspects or embodiments identified herein), the intermediate shaft segment comprises the second shaft.

In an embodiment of the fourth aspect (i.e., independently combinable with any of the other aspects or embodiments identified herein), a separation distance between the first inflatable balloon and the second inflatable balloon is fixed.

In an embodiment of the fourth aspect (i.e., independently combinable with any of the other aspects or embodiments identified herein), a separation distance between the first inflatable balloon and the second inflatable balloon is adjustable.

In an embodiment of the fourth aspect (i.e., independently combinable with any of the other aspects or embodiments identified herein), the catheter further comprises a lumen configured to be placed in fluid communication with a volume of the intravascular environment between the first inflatable balloon and the second inflatable balloon.

In an embodiment of the fourth aspect (i.e., independently combinable with any of the other aspects or embodiments identified herein), the pores are disposed on a central portion of the second inflatable balloon.

In an embodiment of the fourth aspect (i.e., independently combinable with any of the other aspects or embodiments identified herein), the pores are disposed on a distal portion of the second inflatable balloon.

In an embodiment of the fourth aspect (i.e., independently combinable with any of the other aspects or embodiments identified herein), the pores are not disposed on a proximal portion of the second inflatable balloon.

In an embodiment of the fourth aspect (i.e., independently combinable with any of the other aspects or embodiments identified herein), the pores are not disposed on any portion of the second inflatable balloon proximal to a maximum expanded diameter of the balloon in an inflated configuration.

In an embodiment of the fourth aspect (i.e., independently combinable with any of the other aspects or embodiments identified herein), the maximum expanded diameter of the second inflatable balloon is greater than the maximum expanded diameter of the first inflatable balloon.

In an embodiment of the fourth aspect (i.e., independently combinable with any of the other aspects or embodiments identified herein), the length of the expanded second inflatable balloon is greater than the length of the expanded first inflatable balloon.

In an embodiment of the fourth aspect (i.e., independently combinable with any of the other aspects or embodiments identified herein), the catheter further comprises a third inflatable balloon disposed within the interior volume of the second inflatable balloon, the third inflatable balloon having an interior volume in fluid communication with a third inflation lumen.

In an embodiment of the fourth aspect (i.e., independently combinable with any of the other aspects or embodiments identified herein), expansion of the third inflatable balloon is configured to at least partially expand the second inflatable balloon.

In an embodiment of the fourth aspect (i.e., independently combinable with any of the other aspects or embodiments identified herein), expansion of the third inflatable balloon is configured to facilitate expulsion of at least a partial volume of inflation fluid disposed within the interior volume of the second inflatable balloon through the pores into the intravascular environment.

In a fifth aspect (i.e., independently combinable with any of the other aspects or embodiments identified herein), a kit is provided for treating a neuro aneurysm, comprising: an implantable coil; the catheter of the fourth aspect or any of its embodiments; and a hydrolyzer.

In an embodiment of the fifth aspect (i.e., independently combinable with any of the other aspects or embodiments identified herein), the hydrolyzer is ethanol.

In an embodiment of the fifth aspect (i.e., independently combinable with any of the other aspects or embodiments identified herein), the hydrolyzer is dimethyl sulfoxide (DMSO) or contrast media.

In an embodiment of the fifth aspect (i.e., independently combinable with any of the other aspects or embodiments identified herein), the kit further comprises a saline solution.

In an embodiment of the fifth aspect (i.e., independently combinable with any of the other aspects or embodiments identified herein), the PGG has a purity greater than or equal to 99%.

In a sixth aspect (i.e., independently combinable with any of the other aspects or embodiments identified herein), a method is provided for treating a neuro aneurism of a patient, comprising: positioning a first balloon upstream the neuro aneurysm; positioning a second balloon adjacent the neuro aneurysm; inflating the first balloon to occlude downstream blood flow; delivering a therapeutic agent to the site of the aneurysm through pores in the second balloon; and deploying an implantable coil into the aneurism through a delivery catheter supporting the first and/or second balloons.

In an embodiment of the sixth aspect (i.e., independently combinable with any of the other aspects or embodiments identified herein), expanding the second balloon comprises introducing an inflation fluid into an interior volume of the second balloon.

In an embodiment of the sixth aspect (i.e., independently combinable with any of the other aspects or embodiments identified herein), delivering the therapeutic agent comprises introducing a solution comprising the therapeutic agent into an interior volume of the second balloon, the introduction of the solution being configured to expand and/or maintain an expanded state of the second balloon.

In an embodiment of the sixth aspect (i.e., independently combinable with any of the other aspects or embodiments identified herein), inflating the first balloon and expanding the second balloon creates a sealed volume within the blood vessel between the first balloon and the second balloon.

In an embodiment of the sixth aspect (i.e., independently combinable with any of the other aspects or embodiments identified herein), delivering the therapeutic agent comprises introducing the therapeutic agent into the sealed volume.

In an embodiment of the sixth aspect (i.e., independently combinable with any of the other aspects or embodiments identified herein), the therapeutic agent is not delivered into the blood vessel outside of the sealed volume while the sealed volume is established.

In an embodiment of the sixth aspect (i.e., independently combinable with any of the other aspects or embodiments identified herein), inflating the first balloon anchors the first balloon and the second balloon within the blood vessel.

In an embodiment of the sixth aspect (i.e., independently combinable with any of the other aspects or embodiments identified herein), positioning the second balloon adjacent the aneurysm comprises positioning the second balloon across the aneurysm and wherein expanding the second balloon creates a sealed space between the second balloon and the aneurysm.

In an embodiment of the sixth aspect (i.e., independently combinable with any of the other aspects or embodiments identified herein), positioning the second balloon adjacent the aneurysm comprises positioning the second balloon along a downstream edge of the aneurysm and wherein expanding the second balloon creates a sealed volume between the first balloon and the second balloon which encompasses the aneurysm.

In an embodiment of the sixth aspect (i.e., independently combinable with any of the other aspects or embodiments identified herein), positioning the second balloon adjacent the aneurysm comprises positioning the second balloon such that a length of the aneurysm along the blood vessel encompasses an entire length of the second balloon.

In an embodiment of the sixth aspect (i.e., independently combinable with any of the other aspects or embodiments identified herein), inflating the first balloon occurs prior to expanding the second balloon.

In an embodiment of the sixth aspect (i.e., independently combinable with any of the other aspects or embodiments identified herein), expanding the second balloon and/or maintaining the second balloon in an expanded state comprises maintaining a pressure within an interior volume of the second balloon greater than a diastolic blood pressure of the patient and less than a systolic blood pressure of the patient.

In an embodiment of the sixth aspect (i.e., independently combinable with any of the other aspects or embodiments identified herein), expanding the second balloon and delivering the therapeutic agent through the pores comprises introducing a solution into an interior volume of the second balloon, and wherein the solution is introduced at a first volumetric flow rate to expand the second balloon and the solution is introduced at a second volumetric flow rate to deliver the therapeutic agent through the pores, the first volumetric flow rate being greater than or equal to the second volumetric flow rate.

In an embodiment of the sixth aspect (i.e., independently combinable with any of the other aspects or embodiments identified herein), the first volumetric flow rate is greater than the second volumetric flow rate.

In an embodiment of the sixth aspect (i.e., independently combinable with any of the other aspects or embodiments identified herein), blood flow is occluded within the blood vessel for no longer than approximately 3 minutes.

In an embodiment of the sixth aspect (i.e., independently combinable with any of the other aspects or embodiments identified herein), at least 1 mL of solution comprising the therapeutic agent is delivered while downstream blood flow and retrograde blood flow vessel is occluded.

In an embodiment of the sixth aspect (i.e., independently combinable with any of the other aspects or embodiments identified herein), expanding the second balloon comprises inflating a third balloon disposed within an interior volume of the second balloon.

In an embodiment of the sixth aspect (i.e., independently combinable with any of the other aspects or embodiments identified herein), delivering the therapeutic agent comprises inflating a third balloon disposed within an interior volume of the second balloon to force a volume of solution comprising the therapeutic agent within the interior volume of the second balloon through the pores.

In an embodiment of the sixth aspect (i.e., independently combinable with any of the other aspects or embodiments identified herein), the therapeutic agent comprises pentagalloyl glucose (PGG).

In an embodiment of the sixth aspect (i.e., independently combinable with any of the other aspects or embodiments identified herein), the PGG is at least 99.9% pure.

In an embodiment of the sixth aspect (i.e., independently combinable with any of the other aspects or embodiments identified herein), the therapeutic agent is substantially free of gallic acid or methyl gallate.

In a seventh aspect (i.e., independently combinable with any of the other aspects or embodiments identified herein), a method is provided of treating a neuro aneurysm during an open craniotomy, comprising topically applying pentagalloyl glucose (PGG) to the neuro aneurysm or to tissue in a region adjacent to the neuro aneurysm.

Any feature of any aspect or any embodiment is independently combinable, in whole or in part, with one or more other features or aspects as described herein. Any feature of an aspect or embodiment may be made optional to the aspect or embodiment.

### BRIEF DESCRIPTION OF THE DRAWINGS

The features and advantages of the systems, devices, and methods described herein will become apparent from the following description, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1A depicts the chemical structure of 1,2,3,4,6-pentagalloyl glucose (PGG) in a preferred embodiment.

FIG. 1B depicts the chemical structure of gallic acid, a common toxic impurity in the production of PGG.

FIG. 1C depicts the chemical structure of methyl gallate, a common toxic impurity in the production of PGG.

FIGS. 2A-2B schematically depict various examples of a delivery catheter for the delivery of PGG or another therapeutic agent to an endovascular graft or a dissection or an aneurism (peripheral or neuro) or a treatment area in the vicinity thereof. FIG. 2A depicts a delivery catheter in which the balloon is coupled at a proximal end to the distal end of the main shaft. FIG. 2B depicts a delivery catheter in which the balloon is a generally toroidal balloon coupled to the distal end of the main shaft and surrounds the secondary shaft.

FIGS. 2C-2E schematically depict various examples of a delivery catheter for the delivery of PGG or another therapeutic agent to a blood vessel. FIG. 2C depicts a delivery catheter in which the downstream balloon is coupled at a proximal end to the distal end of the main shaft and at the distal end to the secondary shaft, and in which the upstream balloon is coupled to the distal end of the secondary shaft. FIG. 2D depicts a delivery catheter in which the downstream balloon is a generally toroidal balloon coupled to the distal end of the main shaft and surrounds the secondary shaft, and in which the upstream balloon is coupled at proximal and distal ends to the secondary shaft. FIG. 2D also illustrates a supplemental internal lumen in fluid communication with a sealed volume created between the upstream balloon and the downstream balloon and a lead segment positioned on a distal end of the delivery catheter. FIG. 2E depicts a delivery catheter in which the downstream balloon is coupled at proximal and distal ends to the main shaft, and in which the upstream balloon is coupled at proximal and distal ends to the secondary shaft. FIG. 2E also illustrates a secondary shaft having a central lumen which is open at the distal end of the delivery catheter and in fluid communication with the intravascular environment.

FIGS. 3A-3C schematically depict various examples of a downstream balloon of a delivery catheter expanded within a blood vessel comprising an aneurysm. FIG. 3A depicts a downstream balloon that is longer in length than the aneurysm and which is expanded to create a sealed space between the downstream balloon and the blood vessel wall of the aneurysm. FIG. 3A also depicts pores being disposed on a central portion of the downstream balloon. FIG. 3B schematically depicts a downstream balloon expanded to fluidly seal a downstream edge of the aneurysm, creating a sealed volume between the downstream balloon and the upstream balloon (not shown). FIG. 3B also depicts pores being disposed on a distal portion of the downstream balloon. FIG. 3C depicts a downstream balloon that is shorter in length than the aneurysm and which is expanded to bring the downstream balloon into contact with the blood vessel wall of the aneurysm.

FIGS. 4A-4C schematically depict various examples of a delivery catheter comprising an inner balloon disposed within the weeping balloon. FIG. 4A depicts the inner balloon coupled at a proximal end to the distal end of the main shaft. FIG. 4B depicts the inner balloon coupled at proximal and distal ends to the secondary shaft. FIG. 4C depicts the inner balloon coupled at proximal and distal ends to the main shaft.

FIGS. 4D-4F schematically depict various examples of a delivery catheter comprising an inner balloon disposed within the weeping (in some embodiments downstream) balloon. FIG. 4D depicts the inner balloon coupled at a proximal end to the distal end of the main shaft and the weeping balloon coupled at a proximal end to the distal end of the main shaft. FIG. 4E depicts the inner balloon coupled at proximal and distal ends to the distal end of the secondary shaft and the weeping balloon coupled at a proximal end to the distal end of the main shaft. FIG. 4F depicts the inner balloon coupled at proximal and distal ends to the distal end of the main shaft and the weeping balloon coupled at a proximal and distal end to the distal end of the main shaft.

FIGS. 5A-B depict catheters through which a coil is deployed. FIG. 5A schematically depicts an example of a weeping balloon of a delivery catheter through which a coil is deployed. FIG. 5B depicts an example of a balloon of a delivery catheter through which a coil is deployed.

FIG. 6 schematically depicts an example of a balloon of a delivery catheter supporting an endovascular graft or an implantable stent for treatment of a dissection or a stent graft for implantation in a region of a peripheral aneurysm.

### DETAILED DESCRIPTION OF THE PREFERRED EMBODIMENT

### Endovascular Grafts and Associated Placement Procedures

Endovascular aneurysm repair is a type of endovascular surgery that can be used to treat peripheral aneurysms (e.g., of the carotid, femoral, popliteal, or renal artery) or aortic aneurysms. The procedure involves the placement of an endovascular graft within the aorta containing an aneurysm without directly operating on the aorta.

Patients with aortic aneurysms require repair of their aneurysm when it reaches a diameter large enough such that the risk of rupture is greater than the risk of surgery. Repair is also warranted for aortic aneurysms that rapidly enlarge or those that have been the source of emboli (debris from the aneurysm that dislodge and travel into other arteries). Lastly, repair is also indicated for aortic aneurysms that are the source of pain and tenderness, which may indicate impending rupture.

Endovascular aneurysm repair is carried out in a sterile environment under x-ray fluoroscopic guidance. It is usually carried out by an interventional radiologist or sometimes a vascular surgeon or cardiac surgeon, and occasionally, general surgeon or interventional cardiologist. The procedure can be performed under general, regional (spinal or epidural) or even local anesthesia.

Access to the patient's femoral arteries can be with surgical incisions or percutaneously in the groin on both sides. Vascular sheaths are introduced into the patient's femoral arteries, through which guidewires, catheters and the endovascular graft are passed. Diagnostic angiography images are captured to determine the location of the patient's arteries, so the endovascular graft can be properly deployed. The endovascular graft acts as an artificial lumen for blood to flow through, protecting the surrounding aneurysm sac. This reduces the pressure in the aneurysm, which itself will usually thrombose and shrink in size over time.

Leaks can occur in association with endovascular grafts. There are five types of endoleaks, each with different causes and treatment options. A Type I endoleak occurs when there is a gap between the graft and the vessel wall at “seal zones.” The gap allows blood to flow along the side of the graft into the aneurysm sac, which creates pressure within the sac and increases the risk of sac rupture. A Type I endoleak often occurs when the anatomy of the aneurysm is unsuitable for EVAR or inappropriate device selection. However, it can also be caused as the vessel dilates over time. This type of endoleak typically requires urgent attention due to high risk of sac enlargement and rupture. A Type II endoleak results when increased pressure within the side branches of the aorta force blood to leak back into the lower-pressure aneurysm sac. This is the most common type of endoleak, and is generally considered benign. However, it is often unpredictable. A Type III endoleak results from a defect or misalignment between the components of endografts. Similar to what happens with a Type I endoleak, a Type III causes systemic pressure within the aneurysm sac that increases the risk of sac rupture. Therefore, a Type III endoleak also requires urgent attention. A Type IV endoleak occurs soon after some EVAR procedures due to the porosity of certain graft materials. A Type V endoleak, sometimes called endotension, is a poorly understood phenomenon. It is thought to occur when increased graft permeability allows pressure to be transmitted through the aneurysm sac, affecting the native aortic wall.

Conventional treatments for a Type I endoleak include an endovascular procedure to adjust endograft placement so that the “seal zone” shifts to a healthier segment of artery. In some cases, an embolization procedure is used to seal a Type I endoleak. Open surgery is an alternative for individuals who cannot be treated successfully with less invasive techniques. There are multiple therapeutic approaches for type II endoleaks. The most common approach is a translumber embolization. The aneurysm sac is punctured with a needle and clot-inducing materials such as coils or glues are injected. Another common approach is transarterial embolization using microcatheter techniques. Once the microcatheter reaches the area targeted for treatment, clot-inducing materials are released to stop the endoleak. This procedure is often technically demanding, requires extended treatment time and results in higher radiation exposure. Other less commonly used approaches include branch vessel ligation, transgraft embolization and open surgical conversion. A Type III endoleak is commonly treated with additional components to re-line the endograft and seal the defect. Open surgery is an alternative for individuals who cannot be treated successfully with less invasive techniques. A Type IV endoleak often resolves on its own, once blood clotting has normalized. Usually no additional procedures are needed. Treatment of a Type V endoleak is controversial because how it occurs is so poorly understood. Endograft reinforcement procedures have shown promising results, but an open surgery conversion procedure is sometimes necessary.

As noted above, an endovascular graft incorporates a fabric coating that creates a contained tube but is expandable like a bare metal stent. The PGG and/or LeGoo® (a tradename of an internal vessel occluder poloxamer composition produced by Pluromed, Inc.) may be directly coated onto the fabric coating or otherwise impregnated or incorporated into the fabric coating. Alternatively, the stent platform can include a polymer coating (e.g., and/or LeGoo®) that binds to the stent and that releases PGG to the implantation site. For example, one to three or more layers of polymer can be used in the coating, (e.g., a base layer for adhesion), a main layer that holds and elutes (releases) the PGG into the arterial wall by contact transfer, and optionally a top coat to slow down the release of the PGG and extend its effect. The PGG can also be coated directly on the stent platform. Techniques employed in drug-eluting stents can be adapted to incorporating PGG into the endovascular grafts of the embodiments.

### Dissections of the Thoracic or Aortic Artery

A first line treatment for aortic or thoracic dissections is administration of antiplatelet drugs (e.g., aspirin or clopidogrel), blood thinners (e.g., rivaroxaban (Xarelto), apixaban (Eliquis) or edoxaban (Savaysa)), or thrombolytics (e.g., heparin, enoxaparin (Lovenox), dalteparin (Fragmin), fondaparinux (Arixtra), warfarin (Coumadin, Jantoven) or dabigatran (Pradaxa)). Some dissections will heal on their own; however, in certain cases surgical intervention can be necessary, e.g., a surgery to correct the underlying abnormalities that caused the dissection. Other surgical interventions include angioplasty (repairing the dissected section of artery with the aid of inflation of a balloon) or placement of a stent (a mesh-like device that holds the artery open).

A stent is a metal or plastic tube inserted into the lumen of an anatomic vessel or duct to keep the passageway open. Vascular stents are commonly placed as part of the treatment of dissections. Common sites treated with stents include the carotid, iliac, and femoral arteries. Because of the external compression and mechanical forces subjected to these locations, flexible stent materials such as nitinol are used in a majority of peripheral stent placements. A stent graft or covered stent is type of vascular stent with a fabric coating that creates a contained tube but is expandable like a bare metal stent. Covered stents are used in endovascular surgical procedures such as endovascular aneurysm repair, but can also be employed in the treatment of aortic or thoracic dissections.

A drug-eluting stent is one that is placed into an artery to be treated with slow release of a therapeutic drug. Commonly used drugs block cell proliferation. This prevents fibrosis that, together with clots (thrombi), could otherwise block the stented artery, a process called restenosis. The stent is usually placed within the artery by an interventional cardiologist or interventional radiologist during an angioplasty procedure.

As noted above, a stent graft incorporates a fabric coating that creates a contained tube but is expandable like a bare metal stent. The PGG and/or LeGoo® (a tradename of an internal vessel occluder poloxamer composition produced by Pluromed, Inc.) may be directly coated onto the fabric coating or otherwise impregnated or incorporated into the fabric coating. Alternatively, the stent platform can include a polymer coating (e.g., and/or LeGoo®) that binds to the stent and that releases PGG to the implantation site. For example, one to three or more layers of polymer can be used in the coating, (e.g., a base layer for adhesion), a main layer that holds and elutes (releases) the PGG into the arterial wall by contact transfer, and optionally a top coat to slow down the release of the PGG and extend its effect. The PGG can also be coated directly on the stent platform. Techniques employed in drug-eluting stents can be adapted to incorporating PGG into the stent grafts or stents of the embodiments.

Phenolic compounds are a diverse group of materials that have been recognized for use in a wide variety of applications. For instance, they naturally occur in many plants, and are often a component of the human diet. Phenolic compounds have been examined in depth for efficacy as free radical scavengers and neutralizers, for instance in topical skin applications and in food supplements. Phenolic compounds are also believed to prevent cross-linking of cell membranes found in certain inflammatory conditions and are believed to affect the expressions of specific genes due to their modulation of free radicals and other oxidative species (see, for example, U.S. Pat. No. 6,437,004 to Perricone).

What is needed in the art are treatment protocols and compositions for stabilization of the organs and tissues affected by aortic or thoracic dissections, or dissections of other arteries. In particular, treatment protocols utilizing phenolic compounds could provide a safe, less invasive route for the stabilization of the structural architecture in order to temper growth and/or development of such conditions.

### Stent Grafts and Associated Placement Procedures

Conventional treatment for aneurysms in the aorta, arms, legs, or head involve replacing the weakened section of the vessel by a bypass graft that is sutured at the vascular stumps. The graft tube ends, made rigid and expandable by nitinol wireframe, can also be inserted in a reduced diameter into the vascular stumps and then expanded up to the most appropriate diameter and permanently fixed there by external ligature. Such conventional treatments require invasive surgery. S tent grafts have been developed to substitute the external ligature by expandable ring, allowing use in acute ascending aorta dissection, providing airtight (i.e. not dependent on the coagulation integrity), easy and quick anastomosis extended to the arch concavity. Less invasive endovascular techniques allow covered metal stent grafts to be inserted through the arteries of the leg and deployed across the aneurysm.

Endovascular aneurysm repair is a type of endovascular surgery that can be used to treat a peripheral aneurysm, (e.g., the carotid, femoral, popliteal, or renal artery). The procedure involves the placement of an expandable stent graft within the artery with an aneurysm without directly operating on the artery. Endovascular aneurysm repair is appropriate for aneurysms where there exists an adequate length of normal artery for reliable attachment of the endograft without leakage of blood around the device.

Patients with aneurysms require elective repair of their aneurysm when it reaches a diameter large enough such that the risk of rupture is greater than the risk of surgery. Repair is also warranted for aneurysms that rapidly enlarge or those that have been the source of emboli (debris from the aneurysm that dislodge and travel into other arteries). Lastly, repair is also indicated for aneurysms that are the source of pain and tenderness, which may indicate impending rupture.

Endovascular aneurysm repair is carried out in a sterile environment under x-ray fluoroscopic guidance. It is usually carried out by an interventional radiologist or sometimes a vascular surgeon or cardiac surgeon, and occasionally, general surgeon or interventional cardiologist. The procedure can be performed under general, regional (spinal or epidural) or even local anesthesia.

Access to the patient's femoral arteries can be with surgical incisions or percutaneously in the groin on both sides. Vascular sheaths are introduced into the patient's femoral arteries, through which guidewires, catheters and the stent graft are passed. Diagnostic angiography images are captured to determine the location of the patient's arteries, so the stent graft can be properly deployed. The stent graft acts as an artificial lumen for blood to flow through, protecting the surrounding aneurysm sac. This reduces the pressure in the aneurysm, which itself will usually thrombose and shrink in size over time.

As noted above, a stent graft incorporates a fabric coating that creates a contained tube but is expandable like a bare metal stent. The PGG and/or LeGoo® (a tradename of an internal vessel occluder poloxamer composition produced by Pluromed, Inc.) may be directly coated onto the fabric coating or otherwise impregnated or incorporated into the fabric coating. Alternatively, the stent platform can include a polymer coating (e.g., and/or LeGoo®) that binds to the stent and that releases PGG to the implantation site. For example, one to three or more layers of polymer can be used in the coating, (e.g., a base layer for adhesion), a main layer that holds and elutes (releases) the PGG into the arterial wall by contact transfer, and optionally a top coat to slow down the release of the PGG and extend its effect. The PGG can also be coated directly on the stent platform. Techniques employed in drug-eluting stents can be adapted to incorporating PGG into the stent grafts of the embodiments.

### Coils and Associated Placement Procedures

Endovascular aneurysm repair is a type of endovascular surgery that can be used to treat a neuro aneurysm. Endovascular aneurysm repair is appropriate for aneurysms where there exists an adequate length of normal artery for reliable attachment of the endograft without leakage of blood around the device.

Patients with aneurysms require elective repair of their aneurysm when it reaches a diameter large enough such that the risk of rupture is greater than the risk of surgery. Repair is also warranted for aneurysms that rapidly enlarge or those that have been the source of emboli (debris from the aneurysm that dislodge and travel into other arteries). Lastly, repair is also indicated for aneurysms that are the source of pain and tenderness, which may indicate impending rupture.

Endovascular aneurysm repair is carried out in a sterile environment under x-ray fluoroscopic guidance. It is usually carried out by an interventional radiologist or sometimes a vascular surgeon or neuro surgeon, and occasionally, general surgeon. The procedure can be performed under general, regional (spinal or epidural) or even local anesthesia.

Conventional treatment for neuro aneurysm involves coil embolization, a minimally invasive procedure to treat an aneurysm by filling it with material that closes off the sac and reduces the risk of bleeding. It is performed from “within” the artery (endovascular) through a steerable catheter inserted into the blood stream at the groin and guided to the brain. Tiny coils, glue, or mesh stents are used to promote clotting and close off the aneurysm. The goal of endovascular coiling is to isolate an aneurysm from the normal circulation without blocking off any small arteries nearby or narrowing the main vessel. The bloodstream is entered through the femoral artery in the upper leg. A flexible catheter is advanced from the femoral artery to one of four arteries in the neck that lead to the brain. The doctor steers the catheter through the blood vessels while injecting a dye that makes them visible on the monitor.

Once the catheter reaches the aneurysm, a very thin platinum wire is inserted. The wire coils up as it enters the aneurysm and is then detached. Multiple coils can be packed inside the dome to block normal blood flow from entering. Over time, a clot forms inside the aneurysm, effectively removing the risk of aneurysm rupture. Coils remain inside the aneurysm permanently. Coils are made of platinum and other materials, and come in a variety of shapes, sizes, and coatings that promote clotting. Coils accomplish from the inside what a surgical clip would accomplish from the outside: they stop blood from flowing into the aneurysm but allow blood to flow freely through the normal arteries. Aneurysms vary in their size and shape. Saccular aneurysms have a neck at their origin on the main artery and a dome that can expand like a balloon. Other aneurysms, described as wide-necked or fusiform in shape, do not have a defined neck. Placing coils into these aneurysms may be complicated and require additional support from stents or balloons.

### Pentagalloyl Glucose (PGG)

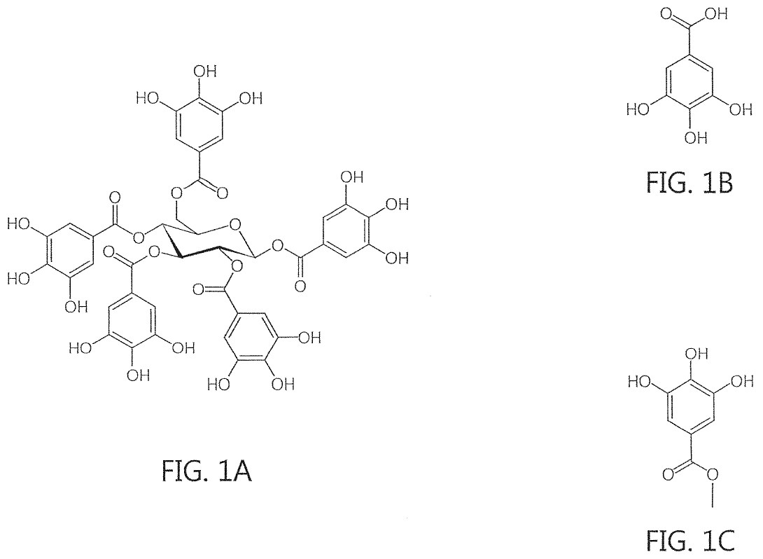

Certain risks associated with endovascular aneurysm repair using endovascular grafts, including treatment of endovascular leaks, or treatment of aortic or thoracic dissections, or peripheral aneurysm treatment using stent grafts, can be mitigated by delivery of pentagalloyl glucose (PGG), e.g., 1,2,3,4,6-pentagalloyl glucose, to the implantation site, to the repair site, or to the surgical site. For example, PGG can be delivered behind an existing stent graft using a microcatheter or a weeping balloon. In preferred embodiments, the PGG may be 1,2,3,4,6-pentagalloyl glucose as depicted in FIG. 1A. However, PGG may refer to any chemical structure encompassed by Formula (I):

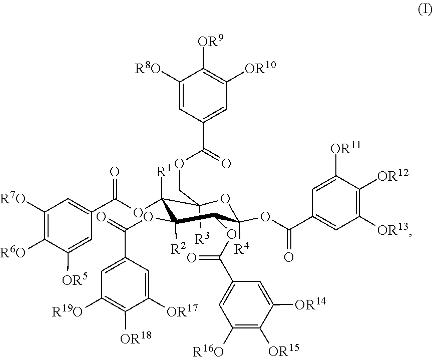

or a pharmaceutically acceptable salt thereof, wherein: R¹-R¹⁹ have any of the values described herein, and wherein the composition is substantially free of gallic acid or methyl gallate. In some embodiments, substantially free is less than about 0.5% gallic acid. In some embodiments, substantially free is less than about 0.5% methyl gallate. In some embodiments, R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl.

Devices for delivery of PGG or another therapeutic agent to the aorta or aortic or thoracic dissection, peripheral aneurysm, neuro aneurysm, surgical site, or implantation site are provided below. Additionally, the devices disclosed herein may be used to delivery any suitable therapeutic agent to the aorta or aortic or thoracic dissection, peripheral aneurysm, neuro aneurysm, surgical site, or implantation site of a subject. PGG may be delivered to a subject to treat aortic aneurysm or an aortic or thoracic dissection or peripheral aneurysm.

In a preferred embodiment, PGG may be delivered to the aorta or aortic or thoracic dissection, peripheral aneurysm, neuro aneurysm, surgical site, or implantation site to stabilize by cross-linking, at least transiently, the elastin proteins within the extracellular matrix of the connective tissue of the aorta wall and implantation site. Treatment of the aorta or aortic or thoracic dissection, peripheral aneurysm, neuro aneurysm, surgical site, or implantation site with an elastin-stabilizing compound, such as PGG, may increase the mechanical integrity of the aorta where the stenosis is present or the artery where the dissection is present or the area where the peripheral aneurysm or neuro aneurysm is present. Treatment with PGG may prevent, inhibit, and/or slow the growth of an aorta aneurysm or an aortic or thoracic dissection or a peripheral aneurysm or a neuro aneurysm. In some instances, treatment with PGG may facilitate natural healing by mechanically stabilizing the aorta. In some implementations, treatment with PGG may be used prior to, after, and/or concurrently with other interventional treatment of an aorta aneurysm, such as surgical intervention. In some implementations, treatment with PGG may be used prior to, after, and/or concurrently with other interventional treatment of an aortic or thoracic dissection, such as surgical intervention, e.g., dissection repair or implantation of a stent. In some instances, treatment with PGG may facilitate natural healing by mechanically stabilizing the peripheral aneurysm or neuro aneurysm. In some implementations, treatment with PGG may be used prior to, after, and/or concurrently with other interventional treatment of a peripheral aneurysm or neuro aneurysm, such as surgical intervention, e.g., stent graft implantation or coil implantation.

In other applications, PGG may be used to treat an aorta aneurysm or aortic or thoracic dissection or peripheral aneurysm or neuro aneurysm using another device or route of administration. For instance, in some embodiments, PGG, particularly a high purity PGG as disclosed herein, may be suitable for direct injection into the bloodstream or into another tissue for treatment of aorta aneurysm or aortic or thoracic dissection or peripheral aneurysm or neuro aneurysm. In some embodiments, PGG may be used to stabilize and/or facilitate closure of the dissection, e.g., an aortic or thoracic dissection, or vascular access holes associated with endovascular graft implantation created by puncturing a blood vessel for therapeutic treatment via the vasculature, such as delivery of a catheter. PGG may promote closure of the vascular access site. The PGG may stabilize the blood vessel wall around the access hole by crosslinking elastin within the blood vessel, which may promote or accelerate natural healing. PGG may be applied to the access hole via intravascular application and/or by applying PGG directly to the skin over the vascular access hole. PGG may have beneficial effects toward wound closure in connective tissue comprising elastin outside the blood vessel wall, such as the superficial layers of skin above the vascular access hole, including subcutaneous tissue. PGG may be used to coat or impregnate the endovascular graft used in the treatment of aorta aneurysm or a graft or stent for treatment of a dissection or peripheral aneurysm or neuro aneurysm. If a surgical procedure is performed to repair the aortic or thoracic dissection, PGG can be applied to the surgically-repaired tissue, the site of the dissection, or tissues in the surgical site or adjacent to the surgical site. In the case of open surgery, this can advantageously be accomplished by administering the PGG in solution form via a syringe to the surgical site. In the case of minimally invasive surgery through the vasculature, a weeping balloon as described herein can be employed to deliver the PGG to the surgical or implantation site.

The concentrations of PGG which may be safely delivered to a patient may be generally proportional to the purity of the PGG. For example, gallic acid, depicted in FIG. 1B, and methyl gallate, depicted in FIG. 1C, are common cytotoxic impurities which may be removed from a source batch of PGG during the purification process. Eliminating the presence of or reducing the concentration of toxic impurities from the delivered PGG may allow higher concentrations of the PGG to be delivered due to the mitigation of the toxic side effects of impurities commonly found in isolated PGG. For instance, studies have shown that substantially 100% pure PGG may be safely delivered at concentrations up to approximately 0.330% (w/v), 95% pure PGG may be safely delivered at concentrations up to approximately 0.125% (w/v), and 85% pure PGG may be safely delivered at concentrations up to approximately 0.06% (w/v). Delivery of PGG in higher concentrations may enhance the amount of uptake of PGG by the target tissue which may increase the efficacy of the PGG treatment. Delivery of PGG in higher concentrations may increase the rate of uptake of PGG by the tissue allowing the same amount of uptake in shorter delivery times. Reducing or minimizing the delivery time may be advantageous for reducing the overall treatment time, and particularly the duration of time for which the aorta is potentially occluded, as described elsewhere herein. Minimization of the treatment time and particularly the duration of blood occlusion may improve the safety and convenience of the treatment procedure and improve patient outcomes.

Unpurified or partially purified PGG may be obtained from any suitable source and purified according to the methods described herein for use as a therapeutic agent. PGG may be extracted from naturally occurring plants such as pomegranate or Chinese gall nut. Extraction and/or isolation methods may entail solvolysis (for example, methanolysis) of tannin or derivative polyphenols as is known in the art. A PGG hydrate is commercially available from Sigma Aldrich (St. Louis, Missouri) at purities greater than or equal to 96%, as confirmed by HPLC. PGG obtained from these sources may undergo additional purification according to the methods described herein to arrive at substantially pure PGG at the purity levels described elsewhere herein.

In some embodiments, PGG is purified by washing a starting batch of PGG (e.g., less than 99% pure) with a solvent. In preferred embodiments, the solvent may comprise diethyl ether. In other embodiments, the solvent may comprise methanol, toluene, isopropyl ether, dichloromethane, methyl tert-butyl ether, 2-butanone, and/or ethyl acetate. In some embodiments, the washing solution may comprise mixtures of the solvents described herein and/or may be mixed with additional solvents. In some embodiments, the starting batch of PGG may be dissolved into a solution. In some embodiments, the PGG may be dissolved in dimethyl sulfoxide (DMSO). In some embodiments, the PGG may be dissolved in any solvent in which the PGG is soluble and which is not miscible with the washing solution. The PGG solution may be mixed with the washing solution in a flask and the PGG solution and washing solution may be allowed to separate over time. The washing solution may subsequently be separated from the PGG solution, such as by draining the denser solution from the flask or by decanting the less dense solution. In some embodiments, the mixture of the washing solution and PGG solution may comprise a volume-to-volume ratio of at least about 1:1, 1.5:1, 2:1, 3:1, 4:1, 5:1, or 10:1 washing solution-to-PGG solution. In some embodiments, the washing step may be repeated at least 1, 2, 3, 4, 5, 6, 7, 8, 9, or 10 times. In some embodiments, the washed PGG solution may be evaporated upon purification to precipitate the PGG into a dry (solid) form. In some embodiments, the PGG may remain dissolved, but the volume of the solution may be increased or decreased (for example, by evaporation). In some embodiments, the starting batch of PGG may be in a dry (solid) form. The PGG may be crystalized. In some embodiments, the PGG may be lyophilized. In some embodiments, the PGG may be precipitated from solution. In some embodiments, the starting batch of PGG may be placed on filter paper and the washing solution poured over the filter paper into a waste flask. The filtration may be facilitated by application of a vacuum to the waste flask (vacuum filtration). Residual washing solution may be evaporated from the purified batch of PGG. In some embodiments, the washing step may be repeated at least 1, 2, 3, 4, 5, 6, 7, 8, 9, or 10 times. The purity of the PGG may increase with each wash. The washing procedure may be repeated until a desired level of purity is attained.

In some embodiments, washing the PGG may result in a purity of at least approximately 99.000%, 99.500%, 99.900%, 99.950%, 99.990%, 99.995%, or 99.999% purity. Purity may be measured as the percent mass (w/w) of PGG in a sample. Purity of the PGG may be measured by any standard means known in the art including chromatography and nuclear magnetic resonance (NMR) spectroscopy. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% gallic acid. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% methyl gallate.

PGG may be prepared in a solution for delivery as a therapeutic agent to a patient. The PGG may comprise a purity described elsewhere herein. The PGG may have been purified by the methods disclosed elsewhere herein or may have been purified by other means. In some embodiments, the PGG may be dissolved in a hydrolyzer for subsequent delivery to a patient. The hydrolyzer may comprise any solvent or mixture of solvents in which PGG is readily soluble and which is miscible with water. In some embodiments, the hydrolyzer may be ethanol. In some embodiments, the hydrolyzer may be dimethyl sulfoxide (DMSO). In some embodiments, the hydrolyzer may be contrast media. In some embodiments, the hydrolyzer may be a mixture of ethanol, DMSO, and/or contrast media in any proportions. The hydrolyzer may facilitate the dissolution of PGG into a larger aqueous solution, in which the PGG would not normally be soluble at the same concentration without first being dissolved into the hydrolyzer. The PGG may ultimately be dissolved into a non-toxic aqueous solution suitable for delivery, such as intravascular delivery, to a patient. The aqueous solution may be a saline solution, as is known in the art, or another aqueous solution comprising salts configured to maintain physiological equilibrium with the intravascular environment. The volumetric ratio of the hydrolyzer to the saline solution may be minimized, while maintaining a sufficient volume of hydrolyzer to fully dissolve the desired amount of PGG, to minimize any harmful or toxic effects of the hydrolyzer on the patient, particularly when delivered intravascularly. In some embodiments, the volume-to-volume ratio of saline to hydrolyzer may be no less than about 10:1, 25:1, 50:1, 75:1, 100:1, 200:1, 300:1, 400:1, 500:1, 600:1, 700:1, 800:1, 900:1, or 1000:1. The total volume of the hydrolyzer and saline mixture (including any other additional components) may be configured to prepare the PGG to a desired therapeutic concentration, such as the concentrations described elsewhere herein. In some embodiments, the PGG may be dissolved into the saline or other aqueous solution without a hydrolyzer. In some embodiments, the saline may be warmed (e.g., to above room temperature or above physiological temperature) to dissolve or help dissolve the PGG (or other therapeutic agent). For instance, the saline may be warmed to at least about 25° C., 30° C., 35° C., 40° C., 45° C., 50° C., 55° C., or 60° C. prior to dissolving the PGG. In some implementations, the therapeutic solution may be raised to and/or maintained at an elevated temperature (e.g., physiological temperature) during delivery.

In some embodiments, PGG (for example, purified PGG) for a therapeutic treatment, including but not limited to those described elsewhere herein, may be provided in a kit comprising the components necessary to prepare the PGG for delivery in a therapeutic solution. In some embodiments, the kit may comprise the PGG in a solid (dry) form, the hydrolyzer, and/or the saline solution. The kit may be configured to optimize the storage conditions of the PGG, for short or long-term storage. In some embodiments, the kit may be configured to store the PGG for up to at least 1 week, 2 weeks, 3 weeks, 4 weeks, 1 month, 2 months, 3 months, 4 months, 5 months, 6 months, 1 year, 2 years, or 3 years. The kit may comprise one or more aliquots of each component in pre-measured amounts or volumes. Each component may be provided in a sealed vial, tube, or other container as is known in the art. The containers may each comprise plastic and/or glass. The containers may be configured (for example, tinted or covered) to protect the components from light and/or other radiation. In some embodiments, the kit may be configured for shipping. For example, the components may be contained in a box or other container including desiccants and/or may be configured for temperature control. In some embodiments, the PGG and/or other components may be supplied in a container that has been purged of air (particularly, oxygen). The component may be stored under vacuum or may be purged with an inert gas, such as nitrogen or argon. In some embodiments, the PGG may be mixed with an antioxidant or other stabilizer, in addition to or alternatively to purging the air. In some embodiments, the antioxidant may comprise Vitamin C, Vitamin E, and/or any other antioxidant or stabilizer which is known in the art and is safe for treatment. In some embodiments, the PGG may be provided already dissolved in the hydrolyzer to a predetermined concentration. In some embodiments, the volume of saline provided may be configured to prepare the PGG at a desired therapeutic concentration. In some embodiments, the volume of saline may be configured to prepare the PGG at a maximal therapeutic concentration, such that a user may dilute the PGG with additional solvent to the desired therapeutic concentration. In some embodiments, the total volume of saline may be configured to prepare the PGG at a concentration below the desired concentration and the user may use only a portion of the volume of the saline to prepare the PGG to the desired concentration. The container of saline may have volume indicators for facilitating measurement of the saline. In some embodiments, the saline may be provided in a plurality of aliquots having the same and/or different volumes, which may allow the user to select an aliquot of a desired volume to prepare the PGG at a desired concentration and/or combine various volumes to prepare the PGG at a desired concentration. In some embodiments, the kit may comprise one or more additional components. For example, the kit may comprise a contrast agent for mixing with the therapeutic PGG solution for allowing indirect visualization of the therapeutic solution, as described elsewhere herein.

### LeGoo®

LeGoo® is a tradename of an internal vessel occluder composition produced by Pluromed, Inc. The composition was given FDA approval in 2011 for temporary endovascular occlusion of blood vessels below the neck up to 4 mm in diameter. The composition was not to be used in patients with vascular anatomy or blood flow that precludes cannula placement or proper injection and control of LeGoo.

LeGoo® is comprised of a 20% (weight percent in saline) of purified poloxamer 407, a non-toxic gel, which is part of a family of biocompatible, water-soluble polymers that possess reverse, thermosensitive properties (i.e. as temperature increases, viscosity increases). Poloxamer 407 dissolves in blood and is excreted in urine. At room temperature it is a viscous but injectable liquid, and it transitions to a temporary self-forming polymeric plug at body temperature. Because the material undergoes a temperature-induced phase change with no alteration in the product's chemical composition, the material does not “cure” in situ.

When used conventionally, LeGoo® is injected into a blood vessel that is intended to be occluded. The amount of LeGoo® injected into the vessel is determined in relationship to the vessel diameter. An arteriotomy is made at a desired location, the cannula is inserted proximally, and LeGoo® is injected against blood flow. When LeGoo® is injected into the blood vessel, the viscosity increases due to the increase in temperature and a plug is formed that occupies space in the vessel, temporarily preventing blood flow. LeGoo® may also be injected distally to stop back bleeding. If left in place and not removed, the plug will dissolve in approximately 15 minutes, or blood flow may be restored by cooling the area with sterile ice or injecting cold saline.

There are two broad categories of vascular occlusion devices available to surgeons to control bleeding: 1. Extravascular occlusion devices; and 2. Intravascular occlusion devices. The mode of action of extravascular occlusive devices is external pressure around the blood vessel. These devices include traditional surgical clamps, clips, vascular (vessel) loops and tapes. The mode of action of intravascular occlusive devices is temporary occlusion of blood flow within a target vessel. Each alternative has its own advantages and disadvantages.

Potential complications may include, but may not be limited to: Effects of transient occlusion of a blood vessel (e.g. infarction, undesired ischemia); Risks associated with the general procedure of clamping a blood vessel (e.g. fibrillation); Risks associated with cannulation (e.g. intimal wall injury.); and Risks associated with application of LeGoo® to epicardial or pericardial surfaces (e.g. adhesions).

LeGoo® is comprised of Poloxamer 407, (also known as Pluronic F127). The conformation of the polymer changes at a certain temperature, the “lower critical solubility temperature” (LCST), or also the “transition temperature.” This conformational change to the somewhat linear polymer allows it to form micelles, which cause an increase in viscosity. If the material is cooled below the transition temperature, then the conformation of the polymer changes back to a somewhat non-linear arrangement and the micelle falls apart. Also, micelles cannot form below a concentration of 12.5%. Once LeGoo® is diluted in blood, the gel plug can no longer occlude the vessel.

Further information about LeGoo® may be in one or more the following U.S. Pat. Nos.: 5,800,711, 6,761,824, 8,043,604, 8,361,455, 8,491,623, 8,821,849, 8,998,928, 9,161,767, each of which is hereby incorporated by reference in its entirety. LeGoo® may be employed as described below, as can other poloxamers exhibiting similar properties of biocompatibility and transition temperature. It is understood that when LeGoo® is referred to herein, that other poloxamers having similar properties may also be employed.

The properties of LeGoo® make it adaptable for use in the treatment of aortic aneurysms or the treatment of aortic or thoracic dissections or in the treatment of peripheral aneurysm or neuro aneurysm. For example, LeGoo® can be employed to occlude a blood vessel (e.g., the aorta) to stop blood flow while an endovascular graft is positioned in a region of an aneurysm (e.g., instead of using a mechanical device such as a balloon to block blood flow). LeGoo® can be employed to occlude a blood vessel (e.g., the aorta) to stop blood flow while a stent or stent graft is positioned in a region of a dissection (e.g., instead of using a mechanical device such as a balloon to block blood flow) or a peripheral aneurysm or when a coil is positioned in the case of neuro aneurysm. In certain embodiments, LeGoo®, optionally containing PGG, can be delivered behind an existing stent graft using a microcatheter or a weeping balloon. In certain embodiments, LeGoo® can be employed as a delivery device for PGG. In such an embodiment, the PGG is mixed with or otherwise combined with LeGoo®, such that the PGG elutes into adjacent tissue in vivo. An endovascular graft can be coated with LeGoo® containing PGG or other therapeutic agents to provide delivery to the tissue in a region of an aneurysm. In other embodiments, LeGoo® containing PGG can be applied to an interior or exterior of a blood vessel, e.g., the aorta, comprising an aneurysm, or to tissue in the region adjacent to the aneurysm, so as to deliver PGG to the tissue. In other embodiments, LeGoo® and/or PGG can be applied to an endovascular graft or a region adjacent to the endovascular graft, e.g., in an endovascular graft implantation procedure or a procedure to repair an endoleak. In certain embodiments, LeGoo® can be employed as a delivery device for PGG. In such an embodiment, the PGG is mixed with or otherwise combined with LeGoo®, such that the PGG elutes into adjacent tissue in vivo. A stent or stent graft or coil can be coated with LeGoo® containing PGG or other therapeutic agents to provide delivery to the tissue in a region of a dissection, peripheral aneurism, or neuro aneurysm. In other embodiments, LeGoo® containing PGG can be applied to an interior or exterior of a blood vessel, e.g., the aorta, comprising a dissection, or to tissue in the region adjacent to the dissection, so as to deliver PGG to the tissue or to provide mechanical stability while a repair of the dissection or associated tissue is performed. A stent graft or coil can be coated with LeGoo® containing PGG or other therapeutic agents to provide delivery to the tissue in a region of an aneurism. In other embodiments, LeGoo® containing PGG can be applied to an interior or exterior of a blood vessel comprising a peripheral or neuro aneurysm, or to tissue in the region adjacent to the aneurysm, so as to deliver PGG to the tissue.

### Delivery Devices

In some implementations, PGG and/or other therapeutic agents or medicaments, including but not limited to those described elsewhere herein, may be delivered to the site of a dissection or an aneurysm, such as an aortic aneurysm or peripheral aneurism or a neuro aneurysm, via a catheter device as described herein, e.g., in the implantation or repair of an endovascular graft or stent or stent graft or coil, e.g., a microcatheter or a weeping balloon catheter. The delivery catheter may be specifically configured (for example, dimensioned), for delivery of a therapeutic agent to the aneurysm in conjunction with placement of an endovascular graft or stent or stent graft, or to a dissection or peripheral aneurysm or neuro aneurysm.

In some embodiments, a balloon may be configured to deliver a therapeutic agent, such as a PGG solution, to the site of an endovascular graft placement or repair or to the implantation or surgical site in a dissection repair or stent or stent graft placement, e.g., in peripheral aneurysm treatment, or coil placement, e.g., in neuro aneurysm. The balloon may be what is known in the art as a weeping balloon. The balloon may comprise a plurality of pores disposed in the expandable membrane of the balloon configured to place the interior volume of the balloon in fluid communication with the intravascular environment. The solution of therapeutic agent may be used as the inflation fluid. The pores may be configured to provide fluid communication between the interior volume of the balloon and the intravascular environment while allowing for pressurization and inflation of the balloon. In some embodiments, the size of the pores may increase as the expandable membrane of the balloon expands. The elastic properties of the expandable membrane of the balloon may allow for a continuous expansion of the pore size of the pores as the interior volume of the balloon is increased causing the expandable membrane to stretch. The volumetric flow rate at which the inflation fluid escapes from the interior volume of the balloon into the intravascular environment may increase as the balloon expands. In some embodiments, the pores may allow for a constant or substantially constant volumetric flow rate of fluid across the pores over a range of pressures of the interior volume. The volumetric flow rate out of the balloon may be maximized at a certain level of pressurization or volumetric flow rates of inflation fluid into the balloon. The inflation fluid may be introduced into the interior volume of the balloon at a volumetric flow rate that is greater than the volumetric flow rate at which the inflation fluid flows through the pores, such that the balloon may be inflated even while fluid escapes or leaks through the pores. In some implementations, the balloon may be inflated using an inflation fluid (for example, saline) that does not comprise the therapeutic agent. The inflation fluid may be switched over to the therapeutic solution or the therapeutic agent may be added to the inflation fluid after the implantation or surgical site has been sealed from retrograde blood flow. Staggering the delivery of the therapeutic agent may conserve the therapeutic agent and/or may prevent, reduce, or minimize the amount of therapeutic agent that is released into the blood stream before the fluid seal is fully formed within the target site or the site of implantation or repair of an endovascular graft or within the surgical site of a dissection or the site of a implantation of a stent or stent graft, e.g., for peripheral aneurism, or implantation of a coil for neuro aneurysm.

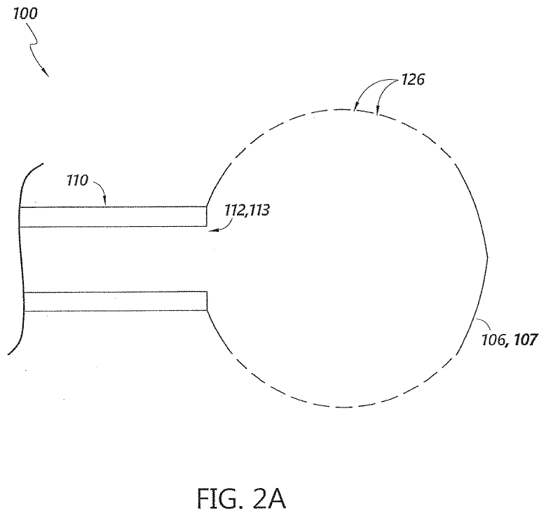

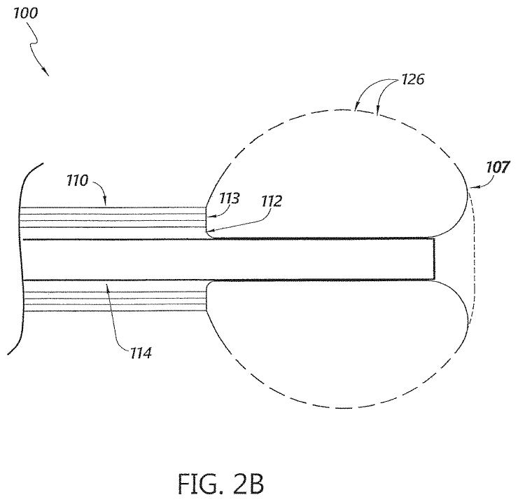

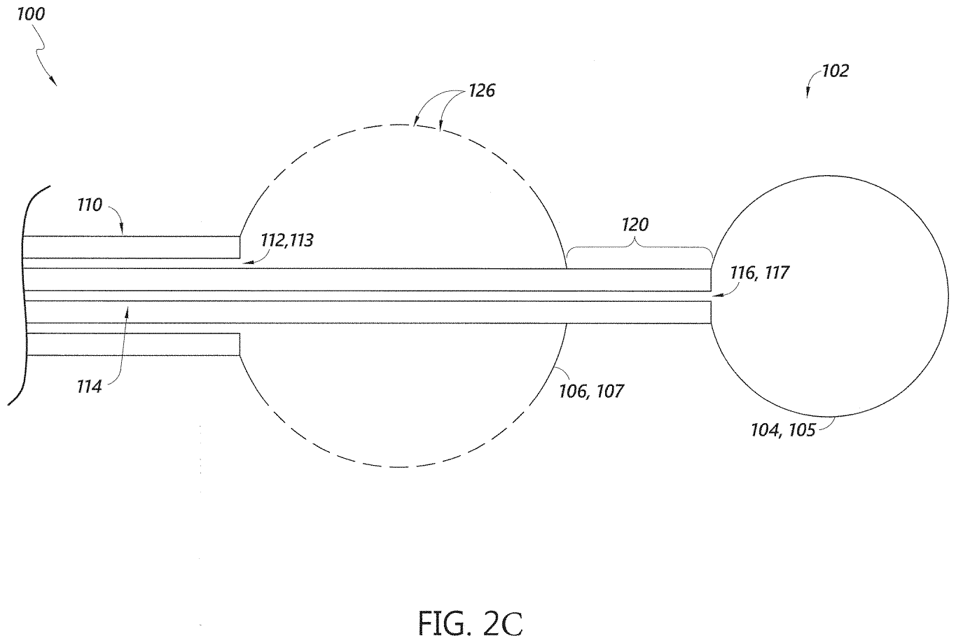

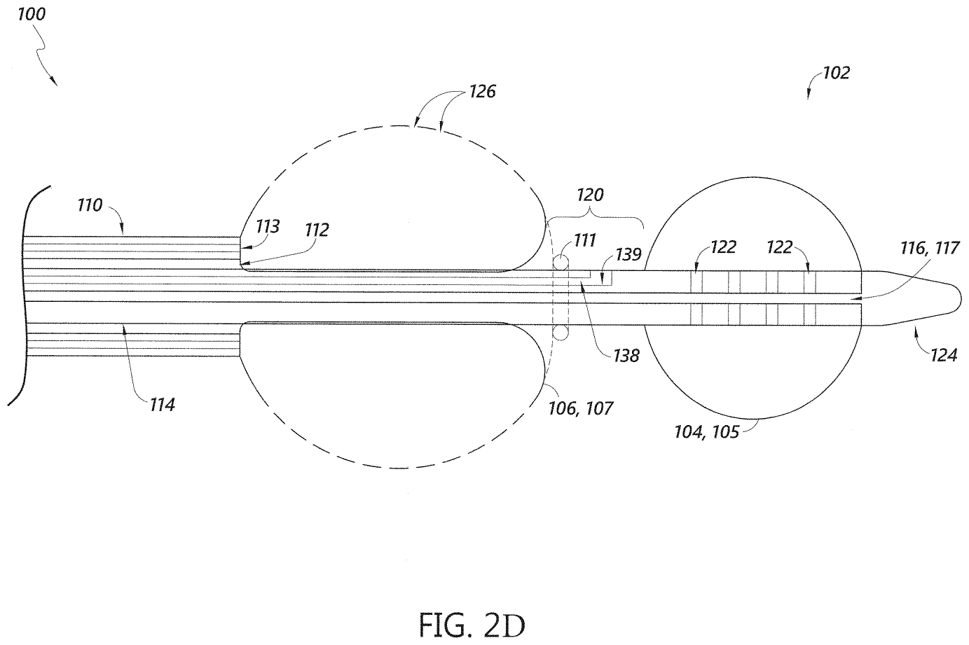

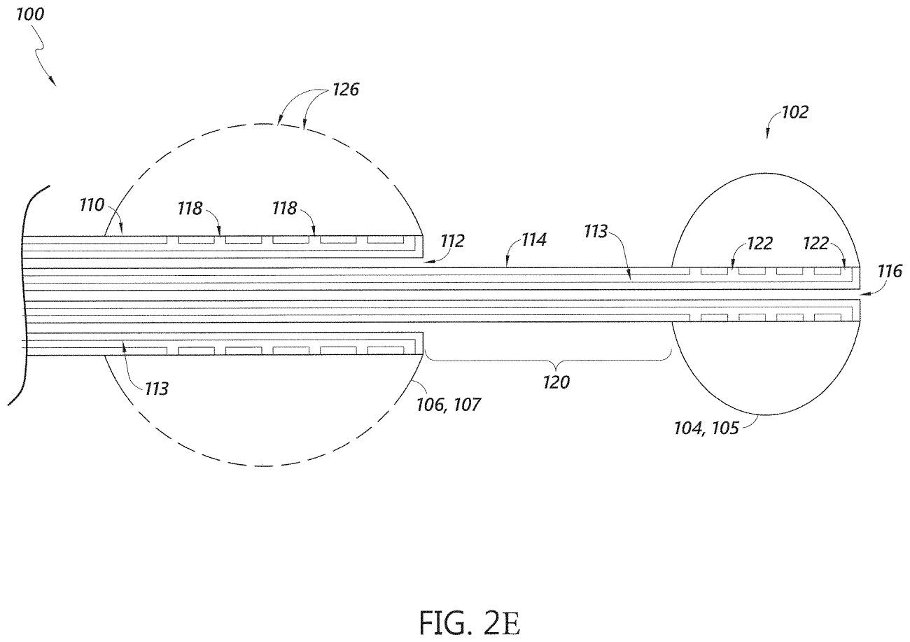

FIG. 2A schematically depicts an example of a weeping balloon. The delivery catheter 100 may comprise a proximal end (not shown), configured to remain outside of the body during use. The delivery catheter 100 may comprise a main shaft 110 and an expandable member 106,107 optionally comprising a plurality of pores 126. Such a configuration is useful for introduction of the delivery catheter 100 from a vascular access point distant from the surgical site. The balloon of FIG. 2A is suitable for use in a balloon angioplasty, or can be adapted to support an implantable endovascular graft, an implantable stent, or stent graft, or to deliver a coil.

The expandable member 106,107 may comprise an expanded configuration having an expanded radial diameter and an unexpanded configuration having an unexpanded radial diameter, the expanded radial diameter being larger than the unexpanded radial diameter. The length of the expandable member 106,107 may increase, decrease, or remain the same upon expansion. The unexpanded diameter of the expandable member 106,107 may be configured to facilitate insertion of the delivery catheter 100 into the artery in the region of the aneurysm, or into the implantation, repair site, or surgical site (for endovascular grafts), or into the artery in the region of the dissection (for balloon angioplasty), or into the implantation site (for stent or stent graft implantation or deployment of a coil) or the surgical site (repair of tissues associated with the dissection). The unexpanded diameters may each be less than, approximately the same as, or larger than an inner diameter and/or outer diameter of the main shaft 110. The expanded diameter of the expandable member 106,107 may be configured to occlude the target site and may be the same as or larger than the diameter of the artery. In some embodiments, the expandable member 106,107 may be operable at intermediate diameters between the unexpanded and fully expanded diameter.

In various embodiments, the expandable member 106,107 may be an inflatable balloon 107, also shown in FIG. 2A. The inflatable balloon 107 may comprise an elastic material forming an expandable membrane as is known in the art and may be configured to expand upon pressurization from an inflation fluid (for example, a gas or a liquid, such as saline). The balloon material may be biocompatible. In some embodiments, the expandable member 106,107 may be expandable through means other than or in addition to inflation. For example, the expandable member 106,107 may comprise a radially expandable frame. The expandable frame may comprise a shape memory material (for example, a nickel titanium alloy (nitinol)) and/or may be configured to self-expand. The expandable member 106,107 may be configured to self-expand upon release of a constraining mechanism, such as an outer sheath surrounding the expandable member, which may, for instance, be proximally withdrawn to allow self-expansion of the expandable member. In some embodiments, the expandable frame may be configured to be mechanically expanded, such as by a push wire or pull wire extending through an internal lumen of the delivery catheter 100. The expandable frames may be fixed or coupled to a surrounding fluid impermeable covering or coating such that the expandable member 106 may be configured to occlude fluid flow as described elsewhere herein.

The main shaft 110 may comprise a length and a diameter configured to facilitate navigation of the expandable member 106,107 to the target site. In some embodiments, the diameter may vary over a length of the main shaft 110 and/or any internal components, including internal shafts described elsewhere herein. For example, the diameter may decrease in a proximal to distal direction causing a distal portion of the delivery catheter 100 to be more flexible than a proximal portion. The main shaft 110 may be generally tubular having a sidewall forming the lumen 112. The lumen 112 may serve as an inflation lumen 113 for inflating and/or deflating the expandable member 106,107. An inflation fluid (for example, saline, for example, containing PGG) may be introduced from a proximal end of the delivery catheter 100 through the inflation lumen 113 into the interior volume of the expandable member 106,107 and removed (for example, aspirated from the expandable member 106,107) through the inflation lumen 113 to de-inflate the expandable member 106,107. The proximal end of the inflation lumen 113 and/or any other inflation lumens described herein may each be in fluid communication with a source of pressurized inflation fluid, such as a syringe, an IV bag, a fluid pump, etc. One or more of the inflation lumens and/or balloons described herein may be in fluid communication with one or more pressure sensors for monitoring pressure levels within the internal lumens and/or the balloons with which they are in fluid communication. In some embodiments in which the expandable member 106,107 comprises an expandable frame, a pull wire or push wire may extend through the first inflation lumen 113 for actuating the expansion or compression of the expandable member 106,107.

In some embodiments, as depicted in FIG. 2A, the balloon 107 may comprise an expandable membrane having a proximal end and a distal end. The proximal end of the expandable membrane may be coupled to (for example, at or near) the distal end of the main shaft 110, e.g., to form fluid-tight seals around the outer diameters of the shaft 110, allowing inflation fluid to pressurize the interior volume of the balloon 107 and the expandable membrane to expand radially outward between the proximal and distal ends of the expandable membrane upon the introduction of the inflation fluid.

In some embodiments, the balloon 107 may have a generally toroidal configuration, as schematically illustrated in FIG. 2B, in which the expandable membrane of the balloon 107 has an outer surface and an inner surface, the inner surface forming a closed circumference defining a central hole through which a secondary shaft 114 may extend. The balloon 107 may define an annular interior volume configured to be pressurized by introduction of inflation fluid from the inflation lumen 113. In some embodiments, the balloon 107 may be coupled to the distal end of the main shaft 110 such that it is in fluid communication with the annular shaped lumen 112 as described with respect to FIG. 2A. In some embodiments, the balloon 107 may be coupled to an outer circumference of the main shaft 110 and in fluid communication with an inflation port formed in the sidewall of the main shaft 110, as described elsewhere herein. In some embodiments, the inner surface of the expandable membrane of the balloon 107 may be coupled to (for example, adhered via an adhesive) an outer diameter of the main shaft 110, the secondary shaft 114, and/or another component of the delivery catheter 100. The balloon of FIG. 2B is suitable for use in delivering a therapeutic agent (e.g., PGG and/or LeGoo®) to an existing endovascular graft (e.g., one that is leaking, e.g., a type I or type II endoleak) or can be adapted to support an endovascular graft for implantation in a target site, or is suitable for use in a balloon angioplasty, or can be adapted to support an implantable stent or stent graft.

In various embodiments, the delivery catheter may combine or interchange the various features illustrated and/or described with respect to FIGS. 2A-2B.

FIG. 2C schematically depicts an example of a delivery catheter 100 that can be employed to deliver a stent graft and/or PGG to the site of a peripheral aneurysm or a coil and/or PGG to the site of a neuro aneurysm. The delivery catheter 100 may comprise a proximal end (not shown), configured to remain outside of the body during use, and a distal end 102, configured to be positioned within the blood vessel near (generally distal to) the target aneurysm or target site or section of blood vessel to be treated. The delivery catheter 100 may comprise a main shaft 110, an upstream expandable member 104,105 and a downstream expandable member 106,107. The delivery catheter 100 may have a longitudinal axis extending from the downstream expandable member 106,107 to the upstream expandable member 104,105. The upstream expandable member 104,105 may be positioned at or near the distal end 102 of the delivery catheter 100 and the downstream expandable member 106,107 may be positioned proximally to the upstream expandable member 104,105. Such a configuration is useful for introduction of the delivery catheter 100 from a vascular access point downstream of the target aneurysm or blood vessel location. For instance, such a configuration is useful for introduction of the delivery catheter 100 through the femoral artery to treat a peripheral or neuro aneurysm. In alternative embodiments, the delivery catheter 100 may be configured for introduction from a location upstream of the target aneurysm or target site of the blood vessel and the upstream expandable member 104 may be positioned proximally to the downstream expandable member 102 with respect to the delivery catheter.

Each expandable member, 104,105 and 106,107, may comprise an expanded configuration having an expanded radial diameter and an unexpanded configuration having an unexpanded radial diameter, the expanded radial diameter being larger than the unexpanded radial diameter. The length of one or both of the expandable members, 104,105 and 106,107, may increase, decrease, or remain the same upon expansion. The unexpanded diameter of each expandable member, 104,105 and 106,107, may be configured to facilitate insertion of the delivery catheter 100 into the blood vessel. The unexpanded diameters may each be less than, approximately the same as, or larger than an inner diameter and/or outer diameter of the main shaft 110. The expanded diameter of each expandable member, 104,105 and 106,107, may be configured to occlude the target blood vessel and may be the same as or larger than the diameter of the target blood vessel (e.g., the carotid, femoral, popliteal, or renal artery). In some embodiments, one or both of the expandable members, 104,105 and 106,107, may be operable at intermediate diameters between the unexpanded and fully expanded diameter. The unexpanded diameter of the upstream expandable member 104,105 may be the same as or different from the unexpanded diameter of the downstream expandable member 106,107. Similarly, the expanded diameter of the upstream expandable member 104,105 may be the same as or different from the expanded diameter of the downstream expandable member 106,107.

In various embodiments, the upstream expandable member 104,105 may be an inflatable balloon 105, as shown in FIG. 2C. In various embodiments, the downstream expandable member 106 may be an inflatable balloon 107, also shown in FIG. 2C. The inflatable balloons 105, 107 may comprise an elastic material forming an expandable membrane as is known in the art and may be configured to expand upon pressurization from an inflation fluid (for example, a gas or a liquid, such as saline). The balloon material may be biocompatible. In some embodiments, the upstream expandable member 104,105 and/or the downstream expandable member 106,107 may be expandable through means other than or in addition to inflation. For example, one or both of the expandable members, 104,105 and 106,107, may comprise radially expandable frames. The expandable frames may comprise a shape memory material (for example, a nickel titanium alloy (nitinol)) and/or may be configured to self-expand. One or both of the expandable members, 104,105 and 106,107, may be configured to self-expand upon release of a constraining mechanism, such as an outer sheath surrounding the expandable member, which may, for instance, be proximally withdrawn to allow self-expansion of the expandable member. In some embodiments, one or both of the expandable frames may be configured to be mechanically expanded, such as by a push wire or pull wire extending through an internal lumen of the delivery catheter 100. The expandable frames may be fixed or coupled to a surrounding fluid impermeable covering or coating such that the expandable members, 104,105 and 106,107, may be configured to occlude fluid flow as described elsewhere herein.

The main shaft 110 of the delivery catheter 100 may extend from the proximal end of the delivery catheter 100 to the downstream balloon 107 (or other downstream expandable member 106,107). The main shaft 110 may comprise a length and a diameter configured to facilitate navigation of the distal end 102 of the delivery catheter 100 to the target site, which may depend on the particular application and/or vascular access site. In some embodiments, the diameter may vary over a length of the main shaft 110 and/or any internal components, including internal shafts described elsewhere herein. For example, the diameter may decrease in a proximal to distal direction causing a distal portion of the delivery catheter 100 to be more flexible than a proximal portion. As shown in FIG. 2C, the downstream balloon 107 may be attached to a distal end of the main shaft 110. The main shaft 110 may have a first central lumen 112. The main shaft 110 may be generally tubular having a sidewall forming the first inflation lumen central lumen 112. The first central lumen 112 may serve as a first inflation lumen 113 for inflating and/or deflating the downstream balloon 107. The first inflation lumen 113 may be in fluid communication with an interior volume of the downstream balloon 107. An inflation fluid (for example, saline) may be introduced from a proximal end of the delivery catheter 100 through the first inflation lumen 113 into the interior volume of the downstream balloon 107 for inflating or expanding the balloon 107 and removed (for example, aspirated from the balloon 107) through the first inflation lumen 113 to de-inflate the balloon 107. The proximal end of the first inflation lumen 113 and/or any other inflation lumens described herein may each be in fluid communication with a source of pressurized inflation fluid, such as a syringe, an IV bag, a fluid pump, etc. One or more of the inflation lumens and/or balloons described herein may be in fluid communication with one or more pressure sensors for monitoring pressure levels within the internal lumens and/or the balloons with which they are in fluid communication. In some embodiments in which the downstream expandable member 106,107 comprises an expandable frame, a pull wire or push wire may extend through the first inflation lumen 113 for actuating the expansion or compression of the downstream expandable member 106,107.

A secondary shaft 114 may extend from a proximal end of the delivery catheter 100 to the upstream balloon 105 (or other upstream expandable member 104,105). As shown in FIG. 2C, the upstream balloon 105 may be attached to a distal end of the secondary shaft 114. In some embodiments, the secondary shaft 114 may extend through the first central lumen 112. The secondary shaft 114 may comprise a secondary central lumen 116. The secondary shaft 116 may be generally tubular having a sidewall forming the secondary central lumen 116. The second central lumen 116 may serve as a second inflation lumen 117 for inflating and/or deflating the upstream balloon 105. The secondary inflation lumen 116 may be in fluid communication with an interior of the upstream balloon 105. An inflation fluid (for example, saline) may be introduced from a proximal end of the delivery catheter 100 through the secondary inflation lumen 117 into the interior volume of the upstream balloon 105 for inflating or expanding the balloon 105 and removed (for example, aspirated from the upstream balloon 105) through the secondary inflation lumen 117 to de-inflate the upstream balloon 105. In some embodiments in which the upstream expandable member 104,105 comprises an expandable frame, a pull wire or push wire may extend through the secondary inflation lumen 117 for actuating the expansion or compression of the upstream expandable member 104,105.

In some embodiments, as shown in FIG. 2C, the secondary shaft 114 may extend through the first central lumen 112. In some embodiments, the secondary shaft 114 may be freely disposed within the first central lumen 112 in a substantially concentric manner. In some embodiments, the secondary shaft 114 may be substantially coaxial with respect to the first central lumen 112. A substantially annular lumen may be formed between the inner diameter of the sidewall of the main shaft 110 and the outer diameter of the sidewall of the secondary shaft 114. Alternatively, the secondary shaft 114 may be coupled to or formed integrally with the inner diameter of the sidewall of the main shaft 110. The distal end of the secondary shaft 114 may extend or be configured to be extendable distally beyond the distal end of the main shaft 110. The secondary shaft 114 may extend through a central portion of the downstream balloon 107 (or other downstream expandable member 106,107).

In some embodiments, as depicted in FIG. 2C, the secondary shaft 114 may extend through the interior of the downstream balloon 107. The downstream balloon 107 may comprise an expandable membrane having a proximal end and a distal end. The proximal end of the expandable membrane may be coupled to (for example, at or near) the distal end of the main shaft 110. The distal end of the expandable membrane may be coupled to the secondary shaft 114 at a point proximal to the upstream balloon 105. The proximal and distal ends of the expandable membrane may be coupled to main shaft 110 and the secondary shaft 114 to form fluid-tight seals around the outer diameters of the shafts 110, 114, allowing inflation fluid to pressurize the interior volume of the downstream balloon 107 and the expandable membrane to expand radially outward between the proximal and distal ends of the expandable membrane upon the introduction of the inflation fluid.

In some embodiments, the downstream balloon 107 (or expandable member 106,107) may have a generally toroidal configuration, as schematically illustrated in FIG. 2D, in which the expandable membrane of the downstream balloon 107 has an outer surface and an inner surface, the inner surface forming a closed circumference defining a central hole through which the secondary shaft 114 may extend. The downstream balloon 107 may define an annular interior volume configured to be pressurized by introduction of inflation fluid from the first inflation lumen 113. In some embodiments, the downstream balloon 107 may be coupled to the distal end of the main shaft 110 such that it is in fluid communication with the annular shaped lumen 112 as described with respect to FIG. 2C. In some embodiments, the downstream balloon 107 may be coupled to an outer circumference of the main shaft 110 and in fluid communication with an inflation port formed in the sidewall of the main shaft 110, as described elsewhere herein. In some embodiments, the generally toroidal downstream balloon 107 may comprise a distal coupling, such as a coupling ring 111, configured to couple a distal end of the downstream balloon 107 to the main shaft 110, the secondary shaft 114, or another component of the delivery catheter 100. The distal coupling may orient the downstream balloon 107 in a proper configuration with respect to the delivery catheter 100. The distal coupling may rigidly fix the downstream balloon 107 to the coupled component (for example, secondary shaft 114) or it may allow the coupled component to axially translate along the longitudinal axis with respect to the distal end of the downstream balloon 107, as described elsewhere herein. In some embodiments, the inner surface of the expandable membrane of the downstream balloon 107 may be coupled to (for example, adhered via an adhesive) an outer diameter of the main shaft 110, the secondary shaft 114, and/or another component of the delivery catheter 100.

In other embodiments, as depicted in FIG. 2E, the main shaft 110 may extend distally to or beyond the distal end of the expandable membrane of the downstream balloon 107. In such embodiments, both the proximal and distal ends of the expandable membrane may be coupled to the main shaft 110. The first inflation lumen 113 may be formed within the sidewall of the main shaft 110 and may be sealed at the distal end to prevent the escape of the inflation fluid. The first inflation lumen 113 may be formed separately from the first central lumen 112. The first inflation lumen 113 may be positioned radially outside of the first central lumen 112. The first central lumen 112 may be configured to receive the secondary shaft 114, as described with respect to FIG. 2C. The main shaft 110 may have one or more inflation ports 118 in fluid communication with the interior volume of the downstream balloon 107 and the first inflation lumen 113. The inflation ports 118 may pass through a sidewall of the main shaft 110. In some embodiments, a plurality of inflation ports 118 may be spaced longitudinally along the main shaft 110 between the proximal and distal ends of the expandable membrane. In some embodiments, a plurality of inflation ports 118 may be spaced radially around the outer diameter of the main shaft 110. The distal end of the main shaft 110 may be positioned at or just beyond the distal end of the downstream balloon 107, as shown in FIG. 2E. In some embodiments, the main shaft 110 may extend to the upstream balloon 105. In some embodiments, the first central lumen 112 may be in fluid communication with a sealed volume 142, described elsewhere herein, formed between the upstream balloon 105 and the downstream balloon 107. In some implementations, the first central lumen 112 may be used to deliver a therapeutic agent into the sealed volume 142 and/or to aspirate fluid from the sealed volume 142, as described elsewhere herein.

The delivery catheter 100 comprises an intermediate shaft segment 120 extending between the downstream balloon 107 and the upstream balloon 105 (or between other expandable members 104,105 and 106,107) and configured to space the upstream balloon 105 distally from the downstream balloon 107. The intermediate shaft segment 120 may connect the upstream balloon 105 and downstream balloon 107. In some embodiments, such as that described with respect to FIG. 2C, the secondary shaft 114 may form the intermediate shaft segment 120 (or at least an outer component of the intermediate shaft segment 120). In some embodiments, the main shaft 110 may form the intermediate shaft segment 120 (or at least an outer component of the intermediate shaft segment 120) or at least a portion of the length of the intermediate shaft segment 120. In some embodiments, a separate tubular connector (not shown) extending from a distal end of the downstream balloon 107 to a proximal end of the upstream balloon 105 may form the outermost component of the intermediate shaft segment 120 and the main shaft 110 and/or secondary shaft 114 may pass through the tubular connector.

The upstream balloon 105 may comprise an expandable membrane. The expandable membrane of the upstream balloon 105 may comprise the same and/or different material(s) as the expandable membrane of the downstream balloon 107. In some embodiments, such as that shown in FIG. 2C, a proximal end of the expandable membrane may be coupled to (for example, at or near) the distal end of the secondary shaft 114 forming a fluid tight seal with the secondary shaft 114. The expandable membrane may not be further coupled to any portion of the delivery catheter 100 distal to the proximal seal, as shown in FIG. 2C, and the upstream balloon 105 may form the distal-most part of the delivery catheter 100. Introduction of inflation fluid into the interior volume of the upstream balloon 105 may cause the upstream balloon 105 to expand radially and distally. In some embodiments, a proximal end of the upstream balloon 105 may be coupled to a shaft positioned concentrically around the secondary shaft 114, such as the main shaft 110 or a separate tubular connector as described elsewhere herein, rather than to the secondary shaft 114 itself. The main shaft 110 or other component to which the upstream balloon 105 is coupled may be fluidly sealed (for example, between an inner diameter of the main shaft 110 and the outer diameter of the secondary shaft 114) such that inflation fluid introduced into an interior volume of the upstream balloon 105 through the secondary inflation lumen 117 may be used to pressurize the upstream balloon 105.

In some embodiments, such as depicted in FIG. 2E, the expandable membrane of the upstream balloon 105 may form a proximal seal and a distal seal with a shaft or shafts of the delivery catheter 100, similar to the downstream balloon 107 as depicted in FIG. 2E. The proximal end of the expandable membrane may be coupled to a proximal point on the secondary shaft 114 and the distal end of the expandable membrane may be coupled to (for example, at or near) the distal end of the secondary shaft 114 at a point distal to the proximal point. The proximal and distal ends of the expandable membrane may be coupled to the secondary shaft 114 to form fluid-tight seals around the outer diameter of the shaft 114, allowing inflation fluid to pressurize the interior volume of the upstream balloon 105 and the expandable membrane to expand radially outward between the proximal and distal ends of the expandable membrane upon the introduction of the inflation fluid. The second inflation lumen 117 may be formed separately from the second central lumen 116, as depicted in FIG. 2E. The second inflation lumen 117 may be positioned radially outside of the second central lumen 116. The second central lumen 116 may be configured to receive additional components such as guidewires, as described elsewhere herein. In other embodiments, as shown in FIG. 2D, the secondary shaft 114 may be sealed at or near its distal end and the second central lumen 116 may serve as the secondary inflation lumen 117. The secondary shaft 114 may have one or more secondary inflation ports 122 in fluid communication with the interior volume of the upstream balloon 105 and the secondary inflation lumen 117. The secondary inflation ports 122 may pass through a sidewall of the secondary shaft 114. In some embodiments, a plurality of secondary inflation ports 122 may be spaced longitudinally along the secondary shaft 114 between the proximal and distal ends of the expandable membrane. In some embodiments, a plurality of secondary inflation ports 122 may be spaced radially around the outer diameter of the secondary shaft 114. The distal end of the secondary shaft 114 may be positioned at or just beyond the distal end of the upstream balloon 105, as shown in FIG. 2E. In some embodiments, as described elsewhere herein, an additional shaft and/or lumen may extend through the second central lumen 116 of the secondary shaft 114 and may extend distally beyond the secondary shaft 114.

In some embodiments, a lead segment 124, such as a rod, may be positioned at a distal end of the delivery catheter 100, as schematically depicted in FIG. 2D. The lead segment 124 may be coupled to or formed from a distal end of the secondary shaft 114 and/or a distal end of the upstream balloon 105. The lead segment 124 may comprise an atraumatic (for example, rounded) distal tip. The lead segment 124 may facilitate the introduction and navigation of the delivery catheter 100 within the vasculature. In some embodiments, the lead segment 124, may comprise a radiopaque material.

In various embodiments, the delivery catheter may combine or interchange the various features illustrated and/or described with respect to FIGS. 2C-2E. For instance, the configurations of the upstream balloon 105 and/or the downstream balloon 107 in each example may be exchanged (or similarly for the expandable members 104,105 and 106,107.

In some embodiments, the upstream balloon 105 may be configured to anchor the delivery catheter 100 within the vasculature when in an expanded configuration, which may include full or partial expansion. Anchoring the delivery catheter 100 within the vasculature may stably position the downstream balloon 107 and/or other portions of the delivery catheter 100 at an appropriate position within the vasculature adjacent an aneurysm or other target site. The upstream balloon 105 may be configured to occlude blood flow (for example, downstream or antegrade blood flow), at least within a sealed volume between the upstream balloon 105 and downstream balloon 107, when in an expanded configuration. The expandable membrane of the upstream balloon 105 may be sufficiently compliant or conformable to assume the shape of and occlude the target vasculature. In some embodiments, the upstream balloon 105 may be configured to occlude the blood vessel in which the peripheral aneurysm is located, e.g., the carotid, femoral, popliteal, or renal artery.

In some embodiments, the downstream balloon 107 may be configured to occlude blood flow (for example, upstream or retrograde blood flow) when in an expanded configuration. In some embodiments, the downstream balloon 107 may be configured to displace blood from the aneurysmal sac of an aneurysm. For example, in some implementations, the downstream balloon 107 may be aligned with an aneurysm (e.g., the length of the aneurysm may encompass the length of the downstream balloon 107) and inflating or expanding the downstream balloon 107 may displace blood from the volume of the aneurysmal sac. Displacing blood from the aneurysmal sac may improve the efficacy of delivering therapeutic agent to an aneurysm (e.g., through the downstream balloon 107). For instance, the therapeutic agent will not be diluted or will be less diluted by blood within the aneurysmal sac. The expandable membrane of the downstream balloon 107 may be sufficiently compliant or conformable to assume the shape of and occlude the target vasculature. In some embodiments, the downstream balloon 107 may be non-compliant (for example, a bag member having a membrane enclosing an expandable interior volume) or less compliant than the upstream balloon 105. In some embodiments, the downstream balloon 107 may be equally compliant relative to the upstream balloon 105. In some embodiments, the downstream balloon 107 may be configured to occlude the blood vessel wherein the peripheral aneurysm is located. In some implementations, the downstream balloon 107 may require a lower threshold pressure to occlude, or fluidly seal, retrograde blood flow if antegrade blood flow has already been stopped. For example, the upstream balloon 105 may require an inflation pressure greater than or equal to the systolic blood pressure to maintain its expanded configuration and the downstream balloon 107 may require a pressure greater than or equal to the diastolic pressure to maintain its expanded configuration. In some implementations, the role of the downstream balloon 107 and the upstream balloon 105 may be reversed, such as if the delivery catheter 100 is introduced from an upstream location.

In some embodiments, the downstream balloon 107 may be configured to deliver a therapeutic agent, such as a PGG solution, to a peripheral aneurysm or other target vasculature site. The downstream balloon 107 may be what is known in the art as a weeping balloon. The downstream balloon 107 may optionally comprise a plurality of pores 126 disposed in the expandable membrane of the balloon configured to place the interior volume of the downstream balloon 107 in fluid communication with the intravascular environment. The solution of therapeutic agent may be used as the inflation fluid. The pores 126 may be configured to provide fluid communication between the interior volume of the downstream balloon 107 and the intravascular environment while allowing for pressurization and inflation of the downstream balloon 107. In some embodiments, the size of the pores 126 may increase as the expandable membrane of the downstream balloon expands. The elastic properties of the expandable membrane of the downstream balloon 107 may allow for a continuous expansion of the pore size of the pores 126 as the interior volume of the downstream balloon 107 is increased causing the expandable membrane to stretch. The volumetric flow rate at which the inflation fluid escapes from the interior volume of the downstream balloon 107 into the intravascular environment may increase as the balloon 107 expands. In some embodiments, the pores 126 may allow for a constant or substantially constant volumetric flow rate of fluid across the pores 126 over a range of pressures of the interior volume. The volumetric flow rate out of the downstream balloon 107 may be maximized at a certain levels of pressurization or volumetric flow rates of inflation fluid into the downstream balloon 107. The inflation fluid may be introduced into the interior volume of the downstream balloon 107 at a volumetric flow rate that is greater than the volumetric flow rate at which the inflation fluid flows through the pores 126, such that the downstream balloon 107 may be inflated even while fluid escapes or leaks through the pores 126. In some implementations, the downstream balloon 107 may be inflated using an inflation fluid (for example, saline) that does not comprise the therapeutic agent. The inflation fluid may be switched over to the therapeutic solution or the therapeutic agent may be added to the inflation fluid after the downstream balloon has been inflated and/or the blood vessel has been sealed from retrograde blood flow. Staggering the delivery of the therapeutic agent may conserve the therapeutic agent and/or may prevent, reduce, or minimize the amount of therapeutic agent that is released into the blood stream before the downstream fluid seal is fully formed with the blood vessel.

In some embodiments, including that shown in FIG. 2C and optionally those shown in FIGS. 2D and 2E, the upstream balloon 105 is connected to the downstream balloon 107 in a fixed spatial relationship, separated by the intermediate shaft segment 120.

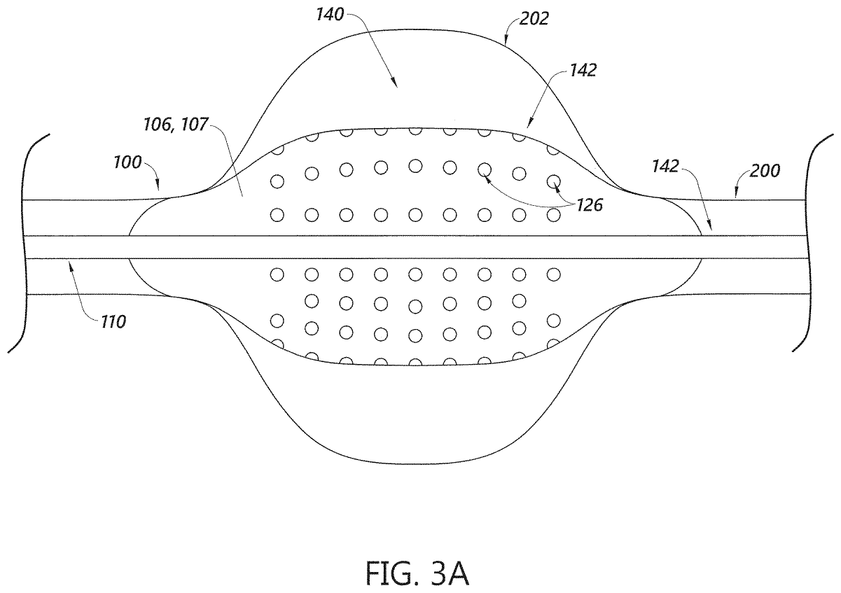

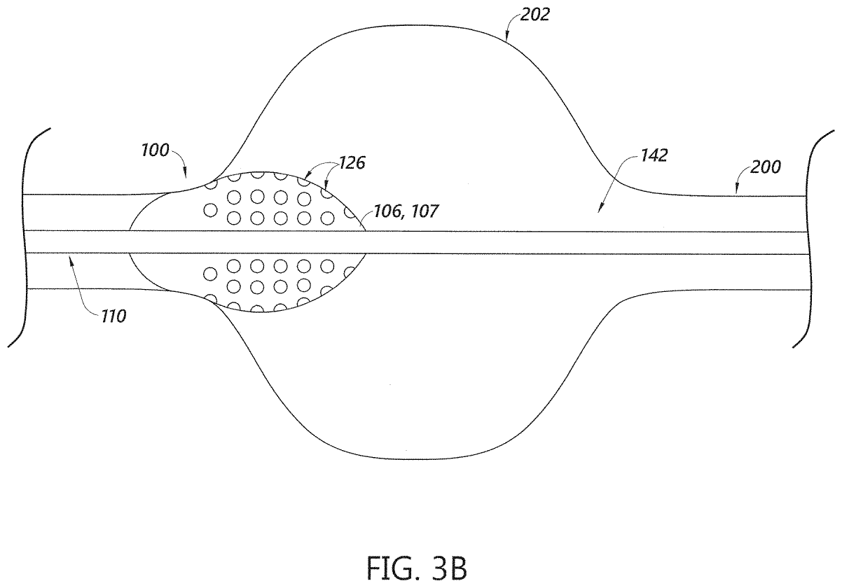

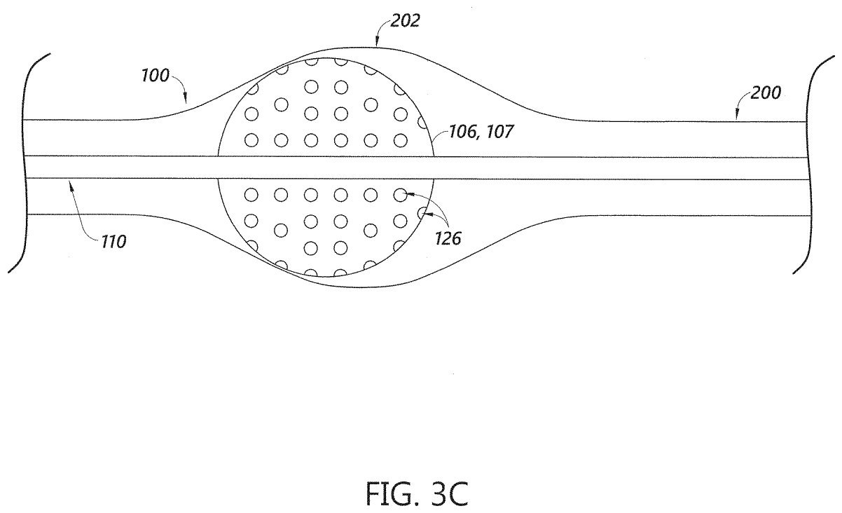

The length of the intermediate shaft segment 120 may be configured to position the downstream balloon 107 a particular distance downstream from the upstream balloon 105. For example, the upstream balloon 105 may be anchored in the blood vessel. The upstream balloon 105 may occlude antegrade and retrograde blood flow from flowing toward the downstream balloon 107. The length of the intermediate shaft segment 120 may be configured to position the downstream balloon 107 near or adjacent to a typical location of a peripheral or neuro aneurysm, as in one of the configurations described with respect to FIGS. 3A-3C. As described elsewhere herein, the delivery catheter 100 may be configured to position the downstream balloon 107 across an aneurysm, if the length of the balloon 107 is substantially the same as or greater than the length of the peripheral aneurysm, or near a downstream edge of the peripheral aneurysm. In some implementations, the length of the downstream balloon 107 may be less than the length of the aneurysm. In some implementations, the size (for example, length) and/or positioning of the downstream balloon 107 (for example, the length of the intermediate shaft segment 120) may depend on the size of the aneurysm and/or the stage of the aneurysm progression. The aneurysm may increase in size (and corresponding length of the blood vessel) over time. A user may select from various configurations of delivery catheters 100 which are configured for aneurysms, e.g., neuro or peripheral, e.g., of different sizes, positions, and/or stages of progression.

In some embodiments, the separation distance of the upstream balloon 105 and the downstream balloon 107 may be adjustable. For example, in the embodiments illustrated in FIGS. 2D and 2E, the secondary shaft 114 may optionally be freely translatable within the main shaft 110 along the longitudinal axis of the delivery catheter 100 such that the distance between the upstream balloon 105 and the downstream balloon 107 is variable and adjustable (for example, continuously or incrementally). The distal end of the main shaft 110 may comprise a sealing feature positioned between an internal diameter of the main shaft 110 and an outer diameter of the secondary shaft 114, which allows the secondary shaft 114 to axially translate (for example, slide) relative to the main shaft 110 while preventing or mitigating fluid flow from the intravascular environment into the first inflation lumen 112. The relative positioning of the main shaft 110 and the secondary shaft 114 may be transiently locked in place by a locking mechanism disposed at the proximal end of the delivery catheter 100. In some embodiments, the secondary shaft 114 may be prevented from advancing distally beyond a distal threshold relative to the main shaft 110 and/or from being retracted proximally beyond a proximal threshold relative to the main shaft 110. For instance, in some embodiments, the upstream balloon 105 may not be configured to be proximally withdrawn past the distal end of the main shaft 110. The upstream balloon 105 may not be configured (for example, dimensioned) to be received within the first central lumen 112. In some embodiments, features at the proximal end of the delivery catheter 100 and/or within the first central lumen 112 between the inner diameter of the main shaft 110 and the outer diameter of the secondary shaft 114 (for example, mechanical catches or latches) may prevent axial translation in the proximal and/or distal direction beyond a certain point.

In some embodiments, the secondary shaft 114 may be removable from the first central lumen 112 of the main shaft 110. The secondary shaft 114 may be reversibly insertable into and removable from the main shaft 110. The secondary shaft 114 may be configured to be removed only when the upstream balloon 105 is in an unexpanded or compressed configuration. In some implementations, the secondary shaft 114 may be inserted into the main shaft 110 and advanced distally beyond the distal end of the main shaft 110 after the main shaft 110 has been navigated to the target site or general target area of the vasculature. In some implementations, the main shaft 110 may be advanced over the secondary shaft 114 after the secondary shaft 114 has been navigated to the target site or general target area of the vasculature. The delivery catheter 100 may be removed from the vasculature after the therapeutic procedure as a single unit or the main shaft 110 or secondary shaft may be removed sequentially in any order. The expandable members 104, 106 may be compressed or unexpanded (for example, the balloons 105, 107 may be deflated) prior to removal of the delivery device 100 or its constituent components from the vasculature.

In some embodiments, one or more of the components of the delivery catheter 100 may comprise radiopaque materials or radiopaque elements (for example, radiopaque rings) may be added to the delivery catheter 100. For example, radiopaque rings may be added to one or more of the distal end of the main shaft 110, the distal end of the secondary shaft 114, the distal and/or proximal ends of the intermediate shaft segment 120, and the upstream or downstream balloons 105, 107 (for example, at proximal and distal ends of the balloons). Use of radiopaque elements or other detectable elements may allow for visual tracking of the delivery catheter within the vasculature, such as through radioscopy or other suitable imaging means, and/or may allow for evaluation of the positioning of the upstream balloon 105 and/or the downstream balloon 107 within the vasculature. In some implementations, the inflation fluid of one or both of the upstream balloon 105 and downstream balloon 107 may include a contrast agent. Use of the contrast agent may allow the user to evaluate the state or amount of inflation of the balloon, may allow the user to determine if the balloon has occluded the blood vessel, and/or, in the case of the downstream balloon 107, may allow the user to monitor the delivery of the therapeutic agent into the blood vessel and/or peripheral aneurysm.

In some embodiments, the delivery catheter 100 may be useable with one or more guidewires for facilitating the introduction and/or navigation of the device into and within the vasculature. In some embodiments, a guidewire may be received within the first central lumen 112, such as when the secondary shaft 114 is removable from the first central lumen 112, and/or a guidewire may be received within the secondary central lumen 116. In some embodiments, the lumen, such as the secondary central lumen 116, may be configured to prevent a guidewire from extending distally beyond a certain point along the length of the lumen. For example, the secondary lumen may be dimensioned with a catch or a tapered or step-down in diameter that prevents the guidewire from extending distally any further. The secondary central lumen 116 may be open or closed at a distal end of the secondary shaft 114. The guidewire may be configured to extend distally beyond the distal end of the secondary shaft 114 in embodiments where the central lumen is open distally to the intravascular environment. In some implementations, the delivery catheter 100 may be introduced over the guidewire after the guidewire has been navigated to or near the target site. In some implementations, the delivery catheter 100 may be capable of being navigated to the target site without use of a guidewire. For example, the delivery catheter 100 may be readily pushed into position via access through the femoral artery without the need for steerability. In some embodiments, the delivery catheter 100 may comprise steerable components, such as the main shaft 110, which may be configured to bend near a distal end 102 of the device. The delivery catheter 100 may comprise one or more pull wires which extend from or from near a distal end 102 of the device to a proximal end of the device. Operation of a control on the proximal end of the delivery catheter 100 may be configured to bend a distal portion of the delivery catheter 100 in one or more directions. Steerability of the delivery catheter 100 may facilitate the introduction and/or navigation of the delivery catheter 100.

In some embodiments, such as that depicted in FIG. 2E, the distal end of the secondary central lumen 116 may be open to the intravascular environment. In some embodiments, the distal end of the main internal lumen 112 may be open, at least partially, to the intravascular environment. In these embodiments, some blood may flow proximally through these lumens across the delivery catheter device. The delivery catheter 100 may be configured such that blood flow through these lumens does not enter the sealed volume 142 between the expanded upstream balloon 105 and expanded downstream balloon 107, as described elsewhere herein. In some embodiments, the blood flow through the internal lumens of the delivery catheter 100 may be in fluid communication with a proximal end of the delivery catheter 100. In some embodiments, the delivery catheter 100 may comprise one or more ports (not shown) in fluid communication with the intravascular environment, positioned proximally to the downstream balloon 107, such that the blood flow, or at least a portion thereof, may be returned to blood vessel downstream of the sealed volume 142. The first central lumen 112 and/or the secondary central lumen 116 may be sealed at a proximal end during use to promote blood flow into the downstream intravascular space rather than through the proximal end of the delivery catheter 100. In some implementations, blood flow through these lumens may be negligible. For instance, the diameter of the lumens may be small enough such that significant volumes of blood are not driven through the lumens during use of the delivery catheter 100. In some implementations, blood flow through these lumens may be non-negligible. In some embodiments, the lumens may be used to maintain blood flow during the procedure and may facilitate prolonging the duration of blood occlusion and the therapeutic treatment.

In some embodiments, the lumens described elsewhere herein may not be formed from the concentric positioning of two or more shafts, but rather may be configured as internal lumens formed as channels within the bodies of one or more unitary shafts. For example, the main shaft 110 may extend from a proximal end of the device, through a center of the downstream balloon 107, to the upstream balloon 105. The main shaft 110 may comprise a plurality of internal lumens (for example, non-concentric lumens) formed within the body material of the main shaft 110. The internal lumens may run substantially parallel to one another. The internal lumens may extend to different lengths along the longitudinal axis of the delivery catheter 100. The internal lumens may be in fluid communication with different components of the delivery catheter 100. For example, one internal lumen may be in fluid communication with the upstream balloon 105 and another internal lumen may be in fluid communication with the downstream balloon 107. The main shaft 110 or other shaft components may comprise additional lumens beyond what is described elsewhere herein. For example, the delivery catheter 100 may have lumens configured for receiving guidewires and/or lumens configured for providing aspiration.

For instance, in some embodiments, the delivery catheter 100 may comprise an aspiration lumen in fluid communication with an aspiration port positioned along the intermediate shaft segment 120. FIG. 2D schematically depicts a supplemental internal lumen 138 in fluid communication with a supplemental fluid port 139 disposed on the intermediate shaft segment 120. The supplemental internal lumen 138 may be used as an aspiration lumen or as a drug delivery lumen, as described elsewhere herein. In some implementations, the aspiration lumen may be used to aspirate the intravascular environment within a sealed volume between the upstream balloon 105 and the downstream balloon 107. Aspiration of fluid (for example, blood) from the sealed volume before and/or during delivery of the therapeutic agent may increase the volume and/or concentration of therapeutic agent that may be delivered to the sealed volume using the delivery device 100. In some implementations, the sealed volume 142 may be aspirated after treatment using the therapeutic agent and before the upstream balloon 105 and/or the downstream balloon 107 is deflated. Removal of the therapeutic agent from the intravascular environment prior to restoring blood flow may eliminate, reduce, or mitigate any downstream and/or non-localized effects of releasing the therapeutic agent into the blood stream. In some embodiments, the supplemental internal lumen 138 in fluid communication with the sealed volume 142 may be used to deliver the therapeutic agent into the sealed volume 142 in addition to or alternatively to a weeping balloon.

The pores 126 of the downstream balloon 107 (or expandable member 106,107) may be disposed uniformly across the surface or a portion of the surface of the downstream balloon 107. In some embodiments, the pores 126 may be disposed in a central portion of the downstream balloon 107 relative to the longitudinal axis. For example, in some embodiments, the length of the downstream balloon 107 may be configured such that the downstream balloon 107 spans the entire length of an aneurysm 202 or target section of a blood vessel 200 and may create a sealed spaced 140 within the aneurysm or section of blood vessel 200 when the downstream balloon 107 is expanded to a minimal diameter, as illustrated in FIG. 3A. The downstream balloon 107 may form a fluid seal with the inner diameter of the blood vessel at points proximal to and/or distal to the aneurysm. The expandable membrane of the downstream balloon 107 may be configured not to expand radially outward into the sealed space 140 between proximal and distal sealing points, to expand partially into the sealed space 140, or to expand entirely into the sealed space 140 such that the outer surface of the downstream balloon 107 conforms to the shape of the aneurysm 202, depending on the properties (for example, elasticity) of the expandable membrane of the downstream balloon 107. In some embodiments, the downstream balloon 107 may be compliant enough to conform to the shape of the aneurysm 202 and blood vessel wall 200, as depicted in FIG. 3A. In some embodiments, the expanded downstream balloon 107 may somewhat expand the diameter of the blood vessel wall proximate to where the downstream balloon 107 forms fluid seals with the aneurysm 202. The pores 126 may be disposed along a central portion configured to be positioned between a proximal fluid seal and a distal fluid seal such that at least a portion of the pores 126 are in fluid communication with the sealed space 140 and allow delivery of the therapeutic inflation fluid into the sealed space 140 or to a tissue within the sealed space. In some embodiments, any remaining pores 126 of the downstream balloon 107 which are not in fluid communication with the sealed space 140 may be disposed in a configuration on the downstream balloon 107 such that the pores 126 are configured to be pressed against the blood vessel 200 wall in an expanded configuration. When the downstream balloon 107 is expanded, the counter pressure of the blood vessel wall against the outer diameter of the downstream balloon 107 may effectively seal the pores 126 in contact with the blood vessel wall from the intravascular environment such that fluid may not flow at any substantial flow rate through those pores 126. This configuration may prevent or minimize delivery of therapeutic agent into non-targeted volumes of the blood vessel and/or into downstream portions of the blood vessel in which the therapeutic agent may be diffused into the bloodstream within the downstream vasculature. In some embodiments, contact between the therapeutic agent within the inflation fluid with the tissue sealed against the pores 126 may be used to treat the blood vessel wall. In some embodiments a plurality of the pores 126 may be spaced at a high density over an area configured to be pressed into contact with the blood vessel wall, such as a portion of the aneurysm. In some embodiments, the pores 126 may be brought into close proximity (for example, no more than 0.3 mm, 0.2 mm, 0.1 mm, 0.075 mm 0.05 mm, 0.025 mm, 0.001 mm, etc.) to the target blood vessel tissue but not into substantial contact, reducing the volume of the sealed space 140 between the expandable membrane of the downstream balloon 107 and the blood vessel wall.

In some embodiments, the pores 126 may be disposed on the downstream balloon 107 (or expandable member 106,107) along a distal portion of the downstream balloon 107, as illustrated in FIG. 3B. The downstream balloon 107 may be positioned and expanded near a proximal edge of an aneurysm or target section of a blood vessel, causing the balloon to form a fluid seal at a proximal edge of the aneurysm or target section or proximal thereto. The distal portion of the downstream balloon 107 on which the pores 126 are disposed may be positioned distally to the proximal fluid seal formed by the downstream balloon 107, such that at least a portion of the pores 126 are in fluid communication with a sealed volume 142 between the proximal seal formed by the downstream balloon 107 and a distal seal formed by the upstream balloon 105. The portion of the downstream balloon 107 proximal to the distal portion may comprise no pores 126 or may comprise less pores 126 than the distal portion. In some embodiments, distal portion may be defined as a portion of the balloon generally distal to a maximum expanded diameter of the downstream balloon 107. Some of the pores 126 may be configured to be pressed against the blood vessel wall where the proximal fluid seal is formed such that the counter pressure of the blood vessel wall effectively seals those pores 126 from the intravascular environment, as described elsewhere herein. This configuration may prevent or minimize delivery of therapeutic agent into downstream portions of the blood vessel in which the therapeutic agent may be diffused into the bloodstream within the downstream vasculature. In some embodiments, the downstream balloon 107 may be configured to be positioned entirely downstream of the aneurysm creating a sealed volume 142 between the upstream balloon 105 and the downstream balloon 107 which confines the aneurysm.

In some embodiments, as schematically illustrated in FIG. 3C, the downstream balloon 107 (or expandable member 106,107) may comprise a length less than a length of the peripheral or neuro aneurysm and may be positioned entirely within the aneurysm. The expanded configuration of the downstream balloon 107 may place the expandable membrane of the downstream balloon in contact with or in close proximity to the blood vessel wall of the aneurysm. The delivery catheter 100 may be configured to position the downstream balloon 107 within the aneurysm, such that a midpoint along the length of the balloon 107 is longitudinally aligned substantially with a midpoint of the peripheral aneurysm or the midpoint of the balloon 107 may be positioned within a proximal or distal portion of the aneurysm. The downstream balloon 107 may be positioned entirely within the length of the aneurysm or the balloon 107 may be positioned partially within the aneurysm and partially outside the aneurysm. In other embodiments, the upstream balloon 105 may be a weeping balloon in addition to or alternatively to the downstream balloon 107 and comprise some or all of the same or similar features as described with respect to the downstream balloon 107.

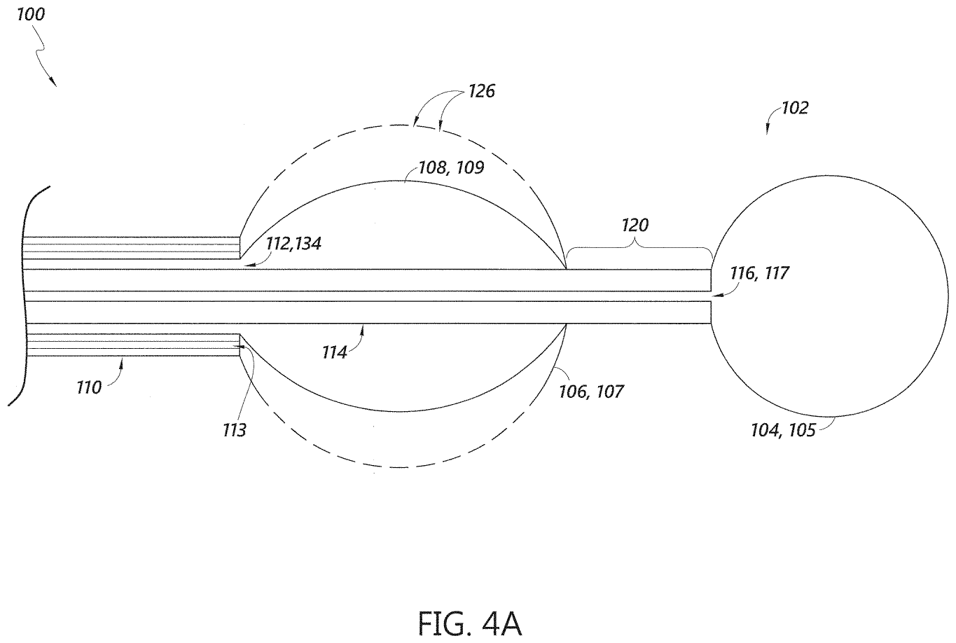

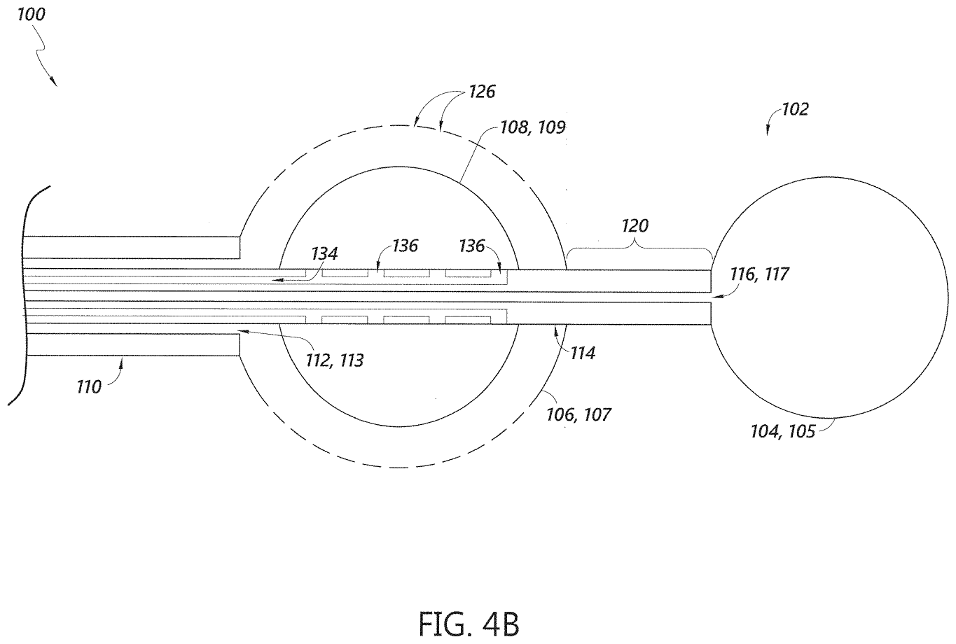

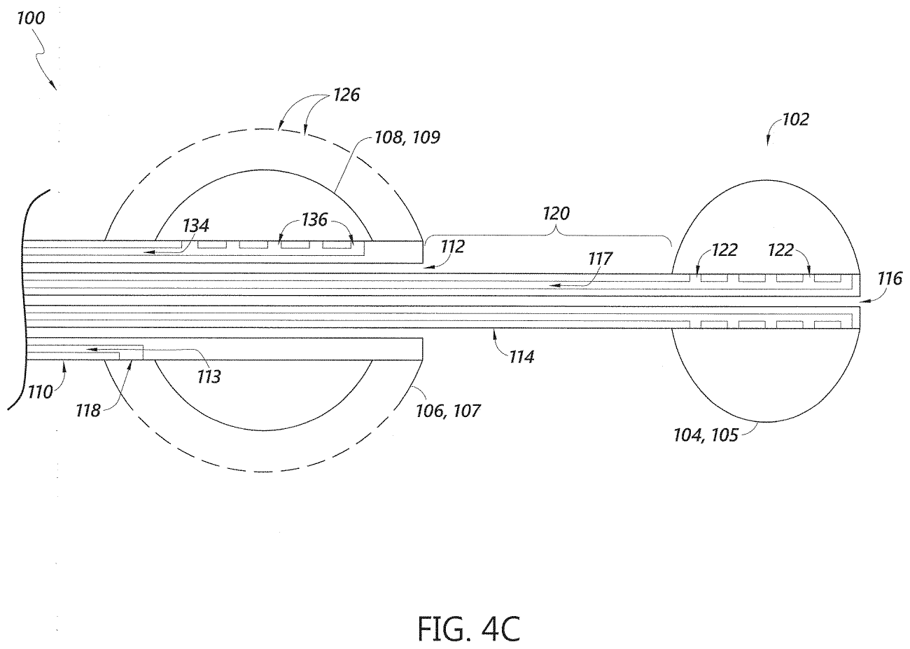

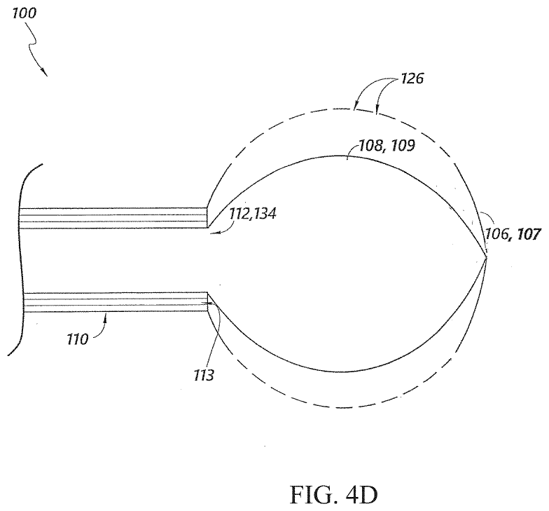

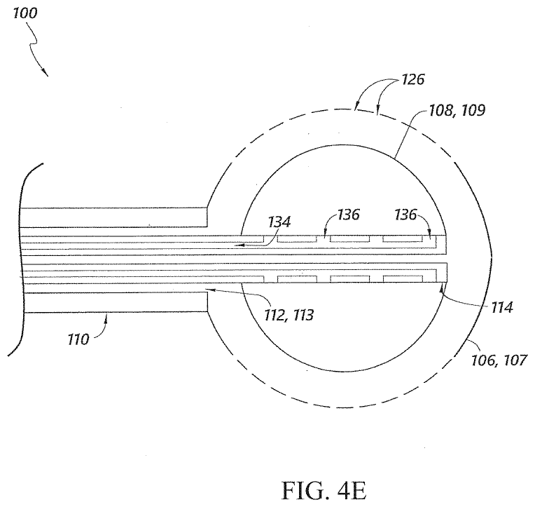

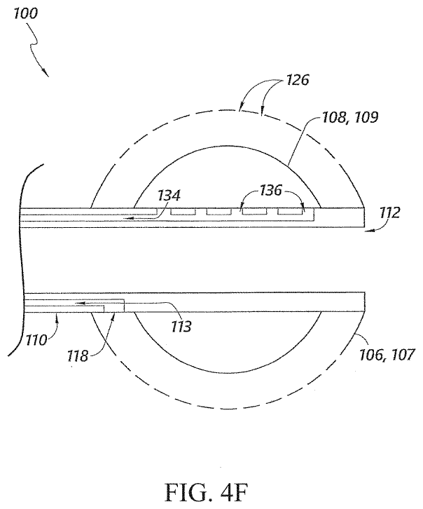

FIGS. 4A-4F schematically illustrate examples of a delivery catheter 100 comprising a third expandable member 108,109. The balloons of FIG. 4A-4C are suitable for use in a balloon angioplasty, or use as a weeping balloon for delivery of a therapeutic agent (e.g., PGG and/or LeGoo®) to a target area (e.g., a leaking endovascular graft), or can be adapted to support an implantable endovascular graft or an implantable stent or stent graft. The third expandable member 108,109 may be an inner balloon 109 as shown in FIGS. 4A-F. FIGS. 4A and 4B may comprise features that are the same or relatively similar to those described with respect to FIG. 2C, and FIG. 4D may comprise features that are the same or relatively similar to those described with respect to FIG. 2A, except for the inclusion of the inner balloon 109. The inner balloon 109 may be positioned entirely within the interior of the downstream balloon 107 (or expandable member 106,107) as shown in FIGS. 4A-4E. The inner balloon 109 may be in fluid communication with a tertiary inflation lumen 134. As shown in FIG. 4C, the tertiary inflation lumen 134 may be formed within the main shaft 110. In some embodiments, the tertiary inflation lumen 134 may be formed radially inside the first inflation lumen 113. The tertiary inflation lumen 134, may be formed by the first central lumen 112, as shown in FIG. 4C. In some embodiments, the tertiary inflation lumen 134 may be formed from a separate tubular component that is carried within the first central lumen 112 of the main shaft 110.

The inner balloon 109 may comprise an expandable membrane. The expandable membrane of the inner balloon 109 may comprise the same and/or different material(s) as the expandable membrane of the downstream balloon 107 and/or the upstream balloon 105. In some embodiments, such as that shown in FIG. 4B, the expandable membrane is coupled to (for example, at or near) the secondary shaft 114 forming a fluid tight seal with the secondary shaft 114 such that an interior volume of the inner balloon 109 may be pressurized. Introduction of inflation fluid into the upstream balloon 105 may cause the inner balloon 109 to expand radially outward between the tertiary inflation lumen 134 and the distal fluid tight seal. The distal end of the expandable membrane of the inner balloon 109 may be substantially longitudinally aligned with the distal end of the expandable membrane of the downstream balloon 107 or may be coupled to the secondary shaft 114 at a point proximal to that where the expandable membrane of the downstream balloon 107 is coupled to the secondary shaft 114.

In some embodiments, as shown in FIG. 4B, proximal and distal ends of the expandable membrane of the inner balloon 109 may be coupled to the secondary shaft 114 to form fluid-tight seals around the outer diameter of the secondary shaft 114. The distal end of the expandable membrane of the inner balloon 109 may be substantially longitudinally aligned with the distal end of the expandable membrane of the downstream balloon 107 or may be coupled to the secondary shaft 114 at a point proximal to that where the expandable membrane of the downstream balloon 107 is coupled to the secondary shaft 114. The proximal end of the expandable membrane of the inner balloon 109 may be substantially longitudinally aligned with the proximal end of the expandable membrane of the downstream balloon 107 or may be coupled to the secondary shaft 114 at a point distal to the proximal end of the downstream balloon 109. Inflation fluid may be introduced to pressurize the interior volume of the inner balloon 109 allowing the expandable membrane to expand radially outward between the proximal and distal ends of the expandable membrane of the inner balloon 109 upon the introduction of the inflation fluid. Inflation fluid may be introduced into the interior of the inner balloon 109 through one or more tertiary inflation ports 136 formed in the sidewall of the secondary shaft 114. The tertiary inflation lumen 134 may be disposed within the secondary shaft 114 rather than the main shaft 110. The tertiary inflation ports 136 may pass through a sidewall of the secondary shaft 114. In some embodiments, a plurality of tertiary inflation ports 136 may be spaced longitudinally along the secondary shaft 114 between the proximal and distal ends of the expandable membrane of the inner balloon 109. In some embodiments, a plurality of tertiary inflation ports 136 may be spaced radially around the outer diameter of the secondary shaft 114.

In some embodiments, as shown in FIG. 4C, the tertiary inflation ports 136 are formed in a sidewall of the main shaft 110 and the inner balloon 109 may be coupled at proximal and distal sealing points to an outer diameter of the main shaft 110. In some embodiments, the inner balloon 109 may be a generally toroidal balloon, as described elsewhere herein with respect to downstream balloon 107. The toroidal inner balloon 109 may be disposed within the interior volume of the downstream balloon 107. In some embodiments, the inner surface of the expandable membrane of the toroidal inner balloon 109 may be coupled at a proximal end, distal end, or along a length or portions of the length of the inner surface to the main shaft 110 or secondary shaft 114, depending on the configuration of the delivery catheter 100. In some embodiments, the inner toroidal balloon 109 may be coupled to the expandable membrane of the downstream balloon 107. In some embodiments, the inner toroidal balloon 109 may be coupled to a shaft and the expandable membrane of the downstream balloon 107. In some embodiments, the toroidal inner balloon 109 may be free-floating within the interior volume of the downstream balloon 107. In some embodiments, the downstream balloon 107 may be a generally toroidal balloon as described elsewhere herein and the inner balloon 109 may be disposed within the annular interior volume of the downstream balloon 107. The generally toroidal inner balloon 109 may be coupled to an inner surface and/or an outer surface of the expandable membrane of the generally toroidal downstream balloon 107 or the inner balloon 109 may be free-floating within the annular interior volume of the downstream balloon 107.

The inner balloon 109 may facilitate the expansion of the downstream balloon 107 and/or the expulsion of inflation fluid (including therapeutic agent) from the downstream balloon 107. The inclusion and inflation of an inner balloon 109 may advantageously reduce the volume of inflation fluid within the downstream balloon 107 necessary to expand the downstream balloon and/or expel inflation fluid through the pores 126 of the downstream balloon 107. The reduction of inflation fluid used within the downstream balloon 109 may conserve the therapeutic agent. The use of the inner balloon 109 may reduce the pressure within the interior of the downstream balloon 107 at which inflation fluid is expelled through the pores 126. In some implementations, a volume of inflation fluid may be introduced into the interior volume of the downstream balloon 107 which is insufficient to fully expand the downstream balloon 107 or to expand the downstream balloon 107 to the inner diameter of the target blood vessel. The inner balloon 109 may be inflated, pressing the volume of inflation fluid within the interior of the downstream balloon 107 against the expandable membrane of the downstream balloon 107 and causing the downstream balloon 107 to expand. In some embodiments, the volume of inflation fluid may be delivered through the pores 126 at a substantial (for example, non-negligible) rate as soon as the combined volume of the inner balloon 109 and the volume of inflation fluid within the downstream balloon 107 is substantially equal to the interior volume of the downstream balloon 107 or as soon as the reduction of volume available for the volume of inflation fluid is small enough that it causes the internal pressure within the downstream balloon 107 to surpass a minimum threshold.

FIGS. 4D-4F schematically illustrate examples of a delivery catheter 100 comprising a second expandable member 108,109. The balloons of FIG. 4D-4F are suitable for use in a balloon angioplasty, or use as a weeping balloon for delivery of a therapeutic agent (e.g., PGG and/or LeGoo®) to a target area (e.g., a leaking endovascular graft), or can be adapted to support an implantable endovascular graft or an implantable stent or stent graft. The second expandable member 108,109 may be an inner balloon 109 as shown in FIG. 4A. FIGS. 4D and 4E may comprise features that are the same or relatively similar to those described with respect to FIG. 2A. The inner balloon 109 may be positioned entirely within the interior of the balloon 105 as shown in FIGS. 4D-4F. The inner balloon 109 may be in fluid communication with a tertiary inflation lumen 134. As shown in FIG. 4C, the tertiary inflation lumen 134 may be formed within the main shaft 110. In some embodiments, the tertiary inflation lumen 134 may be formed radially inside the first inflation lumen 113. The tertiary inflation lumen 134, may be formed by the first central lumen 112, as shown in FIG. 4C. In some embodiments, the tertiary inflation lumen 134 may be formed from a separate tubular component that is carried within the first central lumen 112 of the main shaft 110.

The inner balloon 109 may comprise an expandable membrane. The expandable membrane of the inner balloon 109 may comprise the same and/or different material(s) as the expandable membrane of the balloon 107. In some embodiments, such as that shown in FIG. 4C, the expandable membrane is coupled to (for example, at or near) the secondary shaft 114 forming a fluid tight seal with the secondary shaft 114 such that an interior volume of the inner balloon 109 may be pressurized. Introduction of inflation fluid into the upstream balloon 105 may cause the inner balloon 109 to expand radially outward between the tertiary inflation lumen 134 and the distal fluid tight seal. The distal end of the expandable membrane of the inner balloon 109 may be substantially longitudinally aligned with the distal end of the expandable membrane of the balloon 107 or may be coupled to the secondary shaft 114 at a point proximal to that where the expandable membrane of the balloon 107 is coupled to the secondary shaft 114.

In some embodiments, as shown in FIG. 4E, proximal and distal ends of the expandable membrane of the inner balloon 109 may be coupled to the secondary shaft 114 to form fluid-tight seals around the outer diameter of the secondary shaft 114. The distal end of the expandable membrane of the inner balloon 109 may be substantially longitudinally aligned with the distal end of the expandable membrane of the balloon 107 or may be coupled to the secondary shaft 114 at a point proximal to that where the expandable membrane of the balloon 107 is coupled to the secondary shaft 114. The proximal end of the expandable membrane of the inner balloon 109 may be substantially longitudinally aligned with the proximal end of the expandable membrane of the balloon 107. Inflation fluid may be introduced to pressurize the interior volume of the inner balloon 109 allowing the expandable membrane to expand radially outward between the proximal and distal ends of the expandable membrane of the inner balloon 109 upon the introduction of the inflation fluid. Inflation fluid may be introduced into the interior of the inner balloon 109 through one or more tertiary inflation ports 136 formed in the sidewall of the secondary shaft 114. The tertiary inflation lumen 134 may be disposed within the secondary shaft 114 rather than the main shaft 110. The tertiary inflation ports 136 may pass through a sidewall of the secondary shaft 114. In some embodiments, a plurality of tertiary inflation ports 136 may be spaced longitudinally along the secondary shaft 114 between the proximal and distal ends of the expandable membrane of the inner balloon 109. In some embodiments, a plurality of tertiary inflation ports 136 may be spaced radially around the outer diameter of the secondary shaft 114.

In some embodiments, as shown in FIG. 4F, the tertiary inflation ports 136 are formed in a sidewall of the main shaft 110 and the inner balloon 109 may be coupled at proximal and distal sealing points to an outer diameter of the main shaft 110. In some embodiments, the inner balloon 109 may be a generally toroidal balloon, as described elsewhere herein with respect to balloon 107. The toroidal inner balloon 109 may be disposed within the interior volume of the balloon 107, and an inflation port 118 is in fluid communication with the interior volume of the balloon 107. In some embodiments, the inner surface of the expandable membrane of the toroidal inner balloon 109 may be coupled at a proximal end, distal end, or along a length or portions of the length of the inner surface to the main shaft 110. In some embodiments, the inner toroidal balloon 109 may be coupled to the expandable membrane of the balloon 107. In some embodiments, the inner toroidal balloon 109 may be coupled to a shaft and the expandable membrane of the balloon 107. In some embodiments, the toroidal inner balloon 109 may be free-floating within the interior volume of the balloon 107. In some embodiments, the balloon 107 may be a generally toroidal balloon as described elsewhere herein and the inner balloon 109 may be disposed within the annular interior volume of the balloon 107. The generally toroidal inner balloon 109 may be coupled to an inner surface and/or an outer surface of the expandable membrane of the generally toroidal balloon 107 or the inner balloon 109 may be free-floating within the annular interior volume of the balloon 107.

The inner balloon 109 may facilitate the expansion of the balloon 107 and/or the expulsion of inflation fluid (including therapeutic agent) from the balloon 107. The inclusion and inflation of an inner balloon 109 may advantageously reduce the volume of inflation fluid within the balloon 107 necessary to expand the balloon and/or expel inflation fluid through the pores 126 of the balloon 107. The reduction of inflation fluid used within the inner balloon 109 may conserve the therapeutic agent. The use of the inner balloon 109 may reduce the pressure within the interior of the balloon 107 at which inflation fluid is expelled through the pores 126. In some implementations, a volume of inflation fluid may be introduced into the interior volume of the balloon 107 which is insufficient to fully expand the balloon 107 or to expand the balloon 107 to the inner diameter of the artery, a leaking endovascular graft, or an implantation or target site. The inner balloon 109 may be inflated, pressing the volume of inflation fluid within the interior of the balloon 107 against the expandable membrane of the balloon 107 and causing the balloon 107 to expand. In some embodiments, the volume of inflation fluid may be delivered through the pores 126 at a substantial (for example, non-negligible) rate as soon as the combined volume of the inner balloon 109 and the volume of inflation fluid within the balloon 107 is substantially equal to the interior volume of the balloon 107 or as soon as the reduction of volume available for the volume of inflation fluid is small enough that it causes the internal pressure within the balloon 107 to surpass a minimum threshold.

The delivery catheters of FIGS. 2C-2E and 4A-4C as described above can be modified to omit the downstream balloon, as depicted in FIGS. 2A-B and 4D-F, respectively.

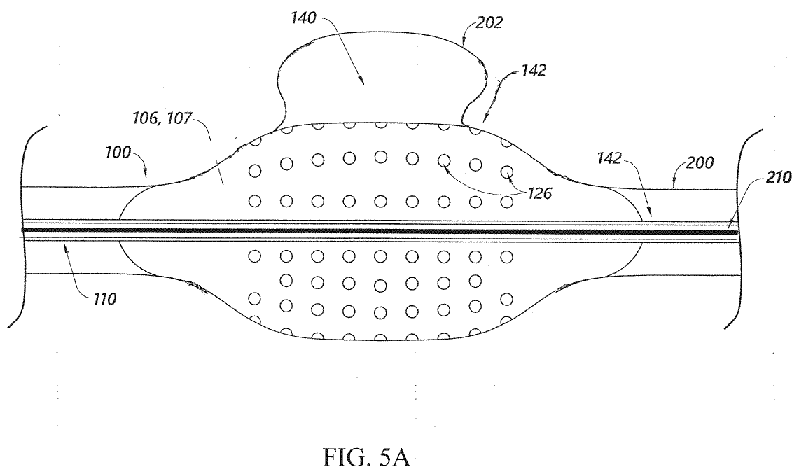

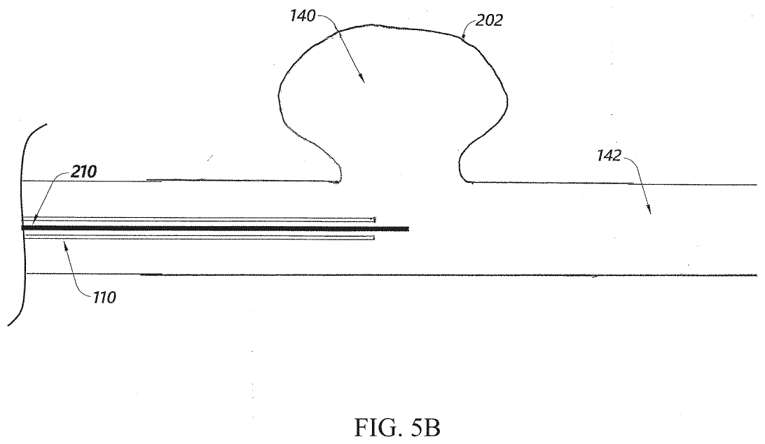

FIG. 5A depicts a modified version of the catheter of FIG. 3A wherein the main shaft 110 further comprises a lumen through which a coil 210 can be passed. With this configuration, before, after, or concurrently with deploying the coil 210 in a neuro aneurysm 202, PGG can be delivered to the neuro aneurysm 202. Any of the other delivery catheters depicted in the figures can be modified to include a lumen adapted to deliver a coil 210 to a neuro aneurysm 202. FIG. 5B depicts a catheter in an absence of a balloon, where a lumen is provided in the main shaft 110 through which the coil 210 advances along with PGG to the site of a neuro aneurysm 202. In other embodiments, the coil 210 is pre-coated or otherwise impregnated with PGG before it is deployed in the delivery catheter.

In some implementations, the delivery catheter 100, described elsewhere herein, or a device having similar features to the delivery catheter 100 may be used to therapeutically treat an aneurysm or a target site of a blood vessel by delivering a therapeutic agent to the peripheral or neuro aneurysm or target site. Described herein is an example of treating a peripheral or neuro aneurysm using the delivery catheter 100 to deliver a therapeutic solution comprising PGG and/or a stent graft, e.g., a stent graft coated or impregnated with PGG, or to deliver a therapeutic solution comprising PGG or an implantable coil coated or impregnated with PGG. Variations of the procedure described herein may be encompassed. In some implementations, a device different from the delivery catheter 100 may be used. In some implementations, a therapeutic other than or in addition to PGG may be delivered. In some implementations, the therapeutic agent may be delivered to another blood vessel or body lumen other than the peripheral or neuro aneurysm. In some implementations, the treatment may be applied for treatment of a blood vessel wall or section of blood vessel that does not comprise a peripheral or neuro aneurysm, is healthy, suffers from a different diseased condition, and/or the therapeutic agent may be intended to be delivered across the blood vessel wall to target the cellular or extracellular environment adjacent the blood vessel.

A method for treating a peripheral or neuro aneurysm is described herein. The method may include or omit any of the steps described elsewhere herein described in relation to the delivery catheter 100. In some embodiments, the delivery catheter 100 is introduced into a femoral artery of the patient. The delivery catheter 100 may be introduced with all expandable members 104,105 and 106,107 (for example, upstream balloon 105 and downstream balloon 107) in an unexpanded configuration. The delivery catheter 100 may be introduced through an optional access sheath. The distal end 102 of the delivery catheter 100 may be navigated into the blood vessel and the upstream balloon 105 positioned at a point upstream of the target peripheral or neuro aneurysm. In some embodiments, a guidewire may be navigated to the target location and the delivery catheter 100 may be introduced over the guidewire as described elsewhere herein. In some embodiments, the delivery catheter 100 may be received over a guidewire and navigated to the target location contemporaneously with the guidewire, using the guidewire to steer the distal end 102 of the delivery catheter 100. In some embodiments, the delivery catheter 100 may be introduced without the use of a guidewire. In some embodiments, the delivery catheter comprises a lumen adapted to deploy a coil, e.g., for treatment of a neuro aneurysm, as depicted in FIG. 5B. The expansion of the upstream balloon 105 partially into the blood vessel may help anchor the balloon. The total procedure time may be sufficiently low (for example, no more than 2-3 min), as described elsewhere herein, such that occlusion of blood flow may safely be maintained during the procedure. The upstream balloon 105 may be expanded with the introduction of inflation fluid into the upstream balloon 105. The upstream balloon 105 may be expanded until the delivery catheter 100 is securely anchored in the blood vessel and/or until the blood flow downstream of the upstream balloon 105 has been occluded. In some embodiments, the operation may be performed under indirect visualization, such as radioscopy. A suitable contrast agent for the method of visualization, (for example, radiocontrast media for radioscopy) may be injected into the blood stream prior to and/or during the operation to visualize blood flow. Accordingly, the occlusion of the blood flow may be visually assessed by indirect visualization.

The downstream balloon 107 may be positioned within, downstream of, or along a downstream edge of the peripheral or neuro aneurysm. In embodiments in which the length of the intermediate shaft segment 120 is adjustable, the delivery catheter 100 may be adjusted to position the downstream balloon 107 in place after the upstream balloon 105 has been anchored in place. The downstream balloon 107 may be expanded with the introduction of inflation fluid into the upstream balloon 105. The downstream balloon 107 may be expanded until retrograde blood flow from downstream of the downstream balloon 107 is occluded. Injection of a contrast agent into the bloodstream may be used to confirm occlusion of blood flow as described elsewhere herein. The inflation of the upstream balloon 105 and downstream balloon 107 may create a fluidly sealed volume 142 within a section of the blood vessel between the two balloons 105, 107. In some implementations, the downstream balloon 107 may be inflated immediately after inflation of the upstream balloon 105 to prevent or minimize the amount of retrograde blood flow into the sealed volume prior to the complete inflation of the downstream balloon 107. In some embodiments, the upstream balloon 105 and the downstream balloon 107 may each be partially inflated, sequentially or simultaneously, and then the upstream balloon 105 may be further expanded to occlude antegrade flow followed by further expansion of the downstream balloon 107 to occlude retrograde flow. In some embodiments, the downstream balloon 107 may be inflated simultaneously with or prior to the inflation of the upstream balloon 105.

In some embodiments, the delivery catheter 100 may comprise an inner balloon 109 positioned within the downstream balloon 107, as described elsewhere herein. In some embodiments, the inner balloon 109 may be partially or fully expanded before inflation fluid is introduced into the downstream balloon 107. In some embodiments, the downstream balloon 107 may be filled with a volume of inflation fluid prior to or simultaneously with the inflation of the inner balloon 109. The first inflation lumen 113 may be configured at a proximal end to prevent unintended proximal flow of inflation fluid due to expansion of the inner balloon 109. For example, an inflation fluid line may be clamped or a pressure may be maintained on a syringe to prevent fluid flow of inflation fluid proximally from the downstream balloon 107 as the inner balloon 109 is expanded. By preventing or inhibiting proximal flow of inflation fluid, expansion of the inner balloon 109 may better promote the expulsion of the volume of inflation fluid within the downstream balloon 107 through the pores 126. In some embodiments, the inflation fluid in communication with the downstream balloon 107 may be switched over to a solution comprising the therapeutic agent after or during expansion of the downstream balloon 107 or the therapeutic agent may be added into the inflation fluid during or after inflation of the downstream balloon 107, as described elsewhere herein. In some embodiments, the initial volume of inflation fluid introduced into the downstream balloon 107 may comprise the therapeutic agent.

Upon inflation of the downstream balloon 107 or the downstream balloon 107 and the inner balloon 109, the inflation fluid, or a partial volume thereof, within the downstream balloon 107 may be expelled through the pores 126, or a portion of the pores 126, into the intravascular environment. The pores 126 may be positioned on a surface of the expandable membrane of the downstream balloon 127 so as to deliver at least some, if not all or a majority of, the delivered inflation fluid into the sealed volume 142 between the upstream balloon 105 and the downstream balloon 107 or a sub-volume thereof. The sub-volume may be a sealed volume (for example, sealed space 140 as shown in FIGS. 5A-5B) formed by the downstream balloon 107 placed in contact with the blood vessel. In embodiments without an inner balloon 109, inflation fluid comprising the therapeutic agent may continue to be supplied to downstream balloon 107 at a pressure or volumetric flow rate configured to maintain the downstream balloon 107 in an expanded configuration after expansion. The delivery device 100 may be configured to provide infusion of the therapeutic agent at a constant pressure. The introduction of therapeutic inflation fluid into the downstream balloon 107 may be maintained long enough to deliver the therapeutic inflation fluid through the pores 126 for a desired duration of time and/or to deliver a predetermined volume of therapeutic inflation fluid through the pores 126. In embodiments comprising an inner balloon 109, the therapeutic inflation fluid may continue to be introduced into the downstream balloon 107 after inflation of the downstream balloon 107 and the inner balloon 109. In some embodiments, the volume of inflation fluid within the downstream balloon 107 may not be replenished as the inner balloon 109 expands to expel the therapeutic inflation fluid through the pores 126.

In some embodiments, the therapeutic agent may be PGG. The PGG may be dissolved in the therapeutic inflation solution at a final concentration that is no less than approximately 0.01%, 0.05%, 0.1%, 0.2%, 0.3%, 0.4%, 0.5%, 0.6%, 0.7%, 0.8%, 0.9%, or 1% (w/v). As described elsewhere herein, higher concentrations of PGG may provide for more effective treatment, especially over shorter treatment times. Accordingly, higher concentrations may allow shorter treatment time. Higher purity PGG may be less toxic, due to absence of toxic impurities, than lower purity PGG. Accordingly, higher purity PGG may be safer to user at higher concentrations than lower purity PGG. The PGG may be dissolved in an inflation fluid such as saline (for example, via a hydrolyzer as described elsewhere herein). The volume of delivered therapeutic inflation fluid may be no more than approximately 150 mL, 125 mL, 100 mL, 75 mL, 50 mL, 40 mL, 30 mL, 20 mL, 15 mL, 10 mL, 8 mL, 5 mL, 3 mL, or 1 mL. In some embodiments, inflation fluid may be delivered through the downstream balloon 107 until a sealed volume, as described elsewhere herein, is filled. In some embodiments, filling of the volume may be detectable by an increase in resistance (a counter pressure) to the delivery of inflation fluid. In some embodiments, filling of the volume may be visually discernable if the inflation fluid comprises a detectable contrast agent. The duration of delivery may be no more than about 30 min, 10 min, 5 min, 4 min, 3 min, 2 min, 1 min, 45 seconds, 30 seconds, 20 seconds, or 10 seconds. The duration of delivery may be shorter in embodiments in which the renal arteries are occluded by the delivery catheter 100. In some implementations, procedures involving occlusions no longer than approximately 10 min may advantageously be performed without the need for general anesthesia. The precise volume of delivered fluid and/or the duration of delivery may depend on the size of the aneurysm or volume of the targeted section of blood vessel to be treated. In some embodiments, the therapeutic inflation solution may be delivered to the downstream balloon 107 at a volumetric flow rate of between approximately 0.05 mL/min and 20 mL/min, 0.1 mL/min and 10 mL/min, 0.5 mL/min and 8 mL/min, or 1 mL/min and 5 mL/min, during the delivery of the therapeutic agent to the blood vessel. In some embodiments, the downstream balloon 107 may be inflated by delivery of inflation fluid at the same volumetric flow rate at which the inflation fluid is introduced during delivery of the therapeutic agent after expansion. In some embodiments, the downstream balloon 107 may be inflated with a volumetric flow rate that is faster than the volumetric flow rate of delivery after expansion. A faster flow rate during expansion of the downstream balloon 105 may facilitate expanding the balloon as the inflation fluid leaks through the pores 126.

By expanding the upstream balloon 107 and occluding downstream blood flow prior to expansion of the downstream balloon 105, the counter pressure needed to cause expansion of the downstream balloon 107 within the intravascular environment may advantageously be reduced. After the downstream blood flow is occluded, the downstream balloon 107 may be expanded upon exceeding the diastolic blood pressure of the patient (for example, approximately 60-80 mmHg), whereas if the downstream blood flow is not occluded, the systolic pressure (for example, approximately 90-120 mmHg) may need to be exceeded. Thus, occluding the downstream blood flow prior to expansion (or full expansion) of the downstream blood flow may facilitate expansion of a weeping balloon, in which pressure may be continually released, such as downstream balloon 107.

In some embodiments, the blood vessel, or a portion thereof (for example, the sealed volume 142 between the upstream balloon 105 and the downstream balloon 107, as depicted in FIG. 5A) may be rinsed prior to or after delivery of the therapeutic agent. A rinsing solution (for example, saline) may be introduced to the intravascular space through the downstream balloon 107 prior to delivery (for example, during expansion as described elsewhere herein) or after delivery of the therapeutic agent. In some embodiments, a rinsing solution may be introduced through a separate internal lumen as described elsewhere herein. For example, a rinsing solution may be introduced into the sealed volume through a fluid port positioned along the intermediate shaft segment 120.

In some embodiments, fluid within the blood vessel, or a portion thereof (for example, the sealed volume 142 between the upstream balloon 105 and the downstream balloon 107, as in FIG. 5A), may be aspirated through the delivery catheter 100. For example, aspiration may be provided through a separate internal lumen through an aspiration port positioned along the intermediate shaft segment 120, as described elsewhere herein. In some embodiments, the sealed volume 142 may be aspirated to remove any blood and/or rinsing solution prior to delivery of the therapeutic agent. In some embodiments, the sealed volume 142 may be rinsed contemporaneously (for example, continuously or intermittently) with the delivery of the therapeutic agent, such that fresh volumes of the therapeutic inflation fluid are introduced into the intravascular space. In some embodiments, the sealed volume 142 may be aspirated to remove the therapeutic agent and/or rinsing solution prior to deflating the upstream balloon 105 and/or downstream balloon 107. Aspiration may advantageously prevent non-targeted delivery of the therapeutic agent to other parts of the blood vessel or body by releasing the therapeutic agent into the blood stream upon deflation of the balloons 105, 107.

Upon completion of the therapeutic treatment and/or delivery of a stent graft, the expandable members 104,105 and 106,107 may be compressed or de-expanded for removal of the delivery catheter 100 from the vasculature. The upstream balloon 105 and downstream balloon 107, and/or the inner balloon 109 may be deflated by withdrawing the inflation fluid proximally through the first inflation lumen 113 and secondary inflation lumen 117, respectively. In some embodiments, the downstream balloon 107 may be deflated, or at least partially deflated, by forcing all or a portion of the inflation fluid through the pores 126 of the expandable membrane without replenishing the inflation fluid within the downstream balloon 107. The upstream balloon 105 may be deflated prior to, after, or substantially simultaneously with the deflation of the downstream balloon 107. The inner balloon 109, when present, may be deflated prior to or substantially simultaneously with the downstream balloon 107. Upon deflation of the balloons, blood flow may be restored downstream of each balloon. The total duration of time for which blood flow is occluded may be no greater than about 30 min, 10 min, 5 min, 4 min, 3 min, 2 min, 1 min, 45 seconds, 30 seconds, 20 seconds, or 10 seconds.

The delivery catheter 100 may be removed from the body by withdrawing the delivery catheter 100 proximally through the vascular access point. In some embodiments in which the delivery catheter 100 comprises multiple components (for example, main shaft 110 and secondary shaft 114 are separable) or is used in conjunction with ancillary components (for example, an access sheath and/or guidewire), the components may be withdrawn in a reverse order in which they were introduced, the components may be withdrawn in a different order, and/or the components or subgroups thereof may be withdrawn contemporaneously. In some embodiments, one or both of the expandable members 104, 106 may need to be placed into an unexpanded configuration or at least partially de-expanded in order to withdraw the delivery catheter 100, e.g., after deployment of the stent graft.

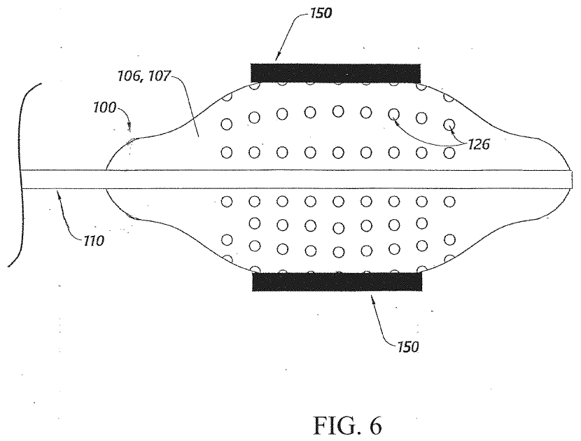

FIG. 6 depicts a modified version of the delivery catheter of FIG. 3A. The delivery catheter of FIG. 6 can support a stent graft 150. After the stent graft is positioned in a peripheral aneurysm, the expandable member (balloon) 106,107 is deflated and the delivery catheter removed, leaving the stent graft 150 in place. Alternatively, the delivery catheter 100 can have a shaft 110 and an expandable member (balloon) 106,107 with a plurality of pores 126, the expandable member (balloon) 106,107 in an inflated form supporting an implantable endovascular graft 150. In the case of treatment of an aneurysm, after the endovascular graft is positioned in the target site, the expandable member (balloon) 106,107 is deflated and the delivery catheter removed, leaving the implantable endovascular graft 150 in place. The balloon of FIG. 3A is adaptable for use in delivering a therapeutic agent (e.g., PGG and/or LeGoo®) to a target site (e.g., a leaking endovascular graft) by omitting the implantable stent, stent graft, or endovascular graft supported thereon. Similarly, the balloons of the other figures can be adapted to support an implantable endovascular graft or an implantable stent or stent graft.

In some embodiments, the balloon 107 may be configured to occlude blood flow (for example, upstream or retrograde blood flow) when in an expanded configuration. In some embodiments, the balloon 107 may be configured to displace blood from the implantation or target site. Displacing blood from the implantation site, surgical site, or target site may improve the efficacy of delivering therapeutic agent to the implantation or target site (e.g., through the balloon 107). For instance, the therapeutic agent will not be diluted or will be less diluted by blood within the implantation site, surgical site, or target site. The expandable membrane of the balloon 107 may be sufficiently compliant or conformable to assume the shape of and occlude the target vasculature. In some embodiments, the balloon 107 may be non-compliant (for example, a bag member having a membrane enclosing an expandable interior volume).

In some embodiments, the balloon 107 may be configured to deliver a therapeutic agent, such as a PGG and/or LeGoo® solution, to an implantation site, surgical site, or target site (e.g., a leaking endovascular graft or dissection). The balloon 107 may comprise a plurality of pores 126 disposed in the expandable membrane of the balloon configured to place the interior volume of the balloon 107 in fluid communication with the environment of the target site. The solution of therapeutic agent may be used as the inflation fluid. The pores 126 may be configured to provide fluid communication between the interior volume of the balloon 107 and the environment of the surgical site while allowing for pressurization and inflation of the balloon 107. In some embodiments, the size of the pores 126 may increase as the expandable membrane of the balloon expands. The elastic properties of the expandable membrane of the balloon 107 may allow for a continuous expansion of the pore size of the pores 126 as the interior volume of the balloon 107 is increased causing the expandable membrane to stretch. The volumetric flow rate at which the inflation fluid escapes from the interior volume of the balloon 107 into the environment of the implantation or target site may increase as the balloon 107 expands. In some embodiments, the pores 126 may allow for a constant or substantially constant volumetric flow rate of fluid across the pores 126 over a range of pressures of the interior volume. The volumetric flow rate out of the balloon 107 may be maximized at a certain level of pressurization or volumetric flow rates of inflation fluid into the balloon 107. The inflation fluid may be introduced into the interior volume of the balloon 107 at a volumetric flow rate that is greater than the volumetric flow rate at which the inflation fluid flows through the pores 126, such that the balloon 107 may be inflated even while fluid escapes or leaks through the pores 126. In some implementations, the balloon 107 may be inflated using an inflation fluid (for example, saline) that does not comprise the therapeutic agent. The inflation fluid may be switched over to the therapeutic solution or the therapeutic agent may be added to the inflation fluid after the balloon has been inflated. Staggering the delivery of the therapeutic agent may conserve the therapeutic agent and/or may prevent, reduce, or minimize the amount of therapeutic agent that is released into the blood stream before the fluid seal is fully formed.

The pores 126 of the balloon 107 may be disposed uniformly across the surface or a portion of the surface of the balloon 107. In some embodiments, the pores 126 may be disposed in a central portion of the balloon 107 relative to the longitudinal axis. For example, in some embodiments, the length of the balloon 107 may be configured such that the balloon 107 spans the entire length of the endovascular graft 150 and may create a sealed space within the artery, dissection, or implantation site, surgical site, or the target site (not illustrated) when the balloon 107 is expanded to a minimal diameter, as illustrated in FIG. 7. The balloon 107 may form a fluid seal with the artery, dissection, or implantation site, surgical site, or the target site. In some embodiments, the balloon 107 may be compliant enough to conform to the shape of the artery, dissection, or implantation site, surgical site, or the target site. In some embodiments, the expanded balloon 107 may somewhat expand the artery in a region of implantation or repair. When the balloon 107 is expanded, the counter pressure of the artery, dissection, or implantation site, surgical site, or the target site against the outer diameter of the balloon 107 may effectively seal the pores 126 from the intravascular environment such that fluid may not flow at any substantial flow rate through those pores 126. This configuration may prevent or minimize delivery of therapeutic agent into non-targeted volumes of the artery or implantation or target site. In some embodiments, contact between the therapeutic agent within the inflation fluid with the tissue or the endovascular graft sealed or stent or stent graft against the pores 126 may be used to treat the tissue in the artery, dissection, or implantation site, surgical site, or the target site. In some embodiments a plurality of the pores 126 may be spaced at a high density over an area configured to be pressed into contact with the artery, dissection, or implantation site, surgical site, or the target site (e.g., a leaking endovascular graft or a dissection). In some embodiments, the pores 126 may be brought into close proximity (for example, no more than 0.3 mm, 0.2 mm, 0.1 mm, 0.075 mm 0.05 mm, 0.025 mm, 0.001 mm, etc.) to the endovascular graft, artery, dissection, or implantation site, surgical site, or the target site but not into substantial contact.

In some embodiments, one or more of the components of the delivery catheter 100 may comprise radiopaque materials or radiopaque elements (for example, radiopaque rings) may be added to the delivery catheter 100. For example, radiopaque rings may be added to one or more of the distal end of the main shaft 110, the distal end of the secondary shaft 114, the distal and/or proximal ends of the intermediate shaft segment 120, the expandable member 106, and the balloon 107 (for example, at proximal and/or distal ends of the balloon). Use of radiopaque elements or other detectable elements may allow for visual tracking of the delivery catheter within the vasculature, such as through radioscopy or other suitable imaging means, and/or may allow for evaluation of the positioning of the balloon 107 within the vasculature. In some implementations, the inflation fluid of the balloon 107 may include a contrast agent. Use of the contrast agent may allow the user to evaluate the state or amount of inflation of the balloon, may allow the user to determine if the balloon has occluded the artery containing a leaking endovascular graft, or an implantation or target site, or the artery containing the dissection or implantation or surgical site, and/or, in the case of the balloon 107, may allow the user to monitor the delivery of the therapeutic agent to the artery, the dissection, or implantation or surgical area—the region of the endovascular graft or the stent or stent graft, or to an implantation or target area.

In some embodiments, the delivery catheter 100 may be useable with one or more guidewires for facilitating the introduction and/or navigation of the device into and within the vasculature. In some embodiments, a guidewire may be received within the first central lumen 112, such as when the secondary shaft 114 is removable from the first central lumen 112. In some embodiments, the lumen may be configured to prevent a guidewire from extending distally beyond a certain point along the length of the lumen. For example, the secondary lumen may be dimensioned with a catch or a tapered or step-down in diameter that prevents the guidewire from extending distally any further. The guidewire may be configured to extend distally beyond the distal end of the secondary shaft 114 in embodiments where the central lumen is open distally to the intravascular environment. In some implementations, the delivery catheter 100 may be introduced over the guidewire after the guidewire has been navigated to or near the target site. In some implementations, the delivery catheter 100 may be capable of being navigated to the target site without use of a guidewire. For example, the delivery catheter 100 may be readily pushed into position via access through the femoral artery without the need for steerability. In some embodiments, the delivery catheter 100 may comprise steerable components, such as the main shaft 110, which may be configured to bend near a distal end of the device. The delivery catheter 100 may comprise one or more pull wires which extend from or from near a distal end of the device to a proximal end of the device. Operation of a control on the proximal end of the delivery catheter 100 may be configured to bend a distal portion of the delivery catheter 100 in one or more directions. Steerability of the delivery catheter 100 may facilitate the introduction and/or navigation of the delivery catheter 100.

In some embodiments, the lumens described elsewhere herein may not be formed from the concentric positioning of two or more shafts, but rather may be configured as internal lumens formed as channels within the bodies of one or more unitary shafts. For example, the main shaft 110 may extend from a proximal end of the device, through a center of the balloon 107. The main shaft 110 may comprise a plurality of internal lumens (for example, non-concentric lumens) formed within the body material of the main shaft 110. The internal lumens may run substantially parallel to one another. The internal lumens may extend to different lengths along the longitudinal axis of the delivery catheter 100. The internal lumens may be in fluid communication with different components of the delivery catheter 100. For example, the internal lumen may be in fluid communication with the balloon 107. The main shaft 110 or other shaft components may comprise additional lumens beyond what is described elsewhere herein. For example, the delivery catheter 100 may have lumens configured for receiving guidewires and/or lumens configured for providing aspiration.

Any or all of the balloons described herein may comprise various shapes. The shapes of the device balloons may be the same or different. In various embodiments, the shape of the balloon may be defined by a surface of revolution. In some embodiments, the balloons may comprise a substantially spherical shape. In some embodiments, the balloons may comprise a spheroid shape, such as a prolate spheroid shape or an oblate spheroid shape. The longitudinal axis of the spheroid may be aligned with the longitudinal axis of the delivery catheter 100. In various embodiments, the length of the balloon may be larger than a diameter of the balloon in its expanded configuration (for example, a prolate spheroid). In some embodiments, the balloons may comprise a pointed football shape. In some embodiments, the balloons may comprise a cylindrical shape. The balloons may comprise distinct proximal and distal surfaces extending from the longitudinal axis of the delivery device 100 to form an edge with an outer surface of the balloon. The proximal and/or distal surfaces may be substantially flat, generally concave, and/or generally convex. The outer surface of the balloons may extend to a diameter greater than, substantially equal to, or less than a diameter of the proximal surface and/or the distal surface. The outer surface may be generally flat, concave, or convex. In some embodiments the pores 126 of the weeping balloon may be only disposed on the outer surface of the balloon or on an outer surface and only one of the proximal and distal surfaces (for example, the distal surface of the balloon 107). In some embodiments, the balloon 107 may comprise one or more inner layers including inner pores. In some embodiments, the inner pores may generally comprise diameters greater than or equal to the diameter of the pores 126. The inner pores may serve as baffles which may help facilitate uniform distribution of the inflation fluid (and therapeutic agent) within the interior of the balloon 107.

The outer diameter of the balloon 107 in an expanded configuration (for example, at its widest point) may be sized to a diameter of at least approximately 1.5 cm, 1.75 cm, 2.0 cm, 2.25 cm, 2.5 cm, 3.0 cm, 3.5 cm, or 4.0 cm or more. The outer diameter of the balloon 107 in an expanded configuration may be configured to match or slightly exceed the diameter of a healthy artery or an implanted endovascular graft. In some embodiments, the balloon 107 may be configured to expand to the diameter of a healthy artery or an implanted endovascular graft or slightly exceed the diameter of a healthy artery or an implanted endovascular graft such that it may form a fluid seal downstream and/or upstream of the target site, or to the diameter of a healthy artery such that it may form a fluid seal downstream and/or upstream of the dissection or implantation or surgical site. In some embodiments, the total volume of the balloon 107 (for example, in an expanded configuration) or of the holding capacity of deliverable fluid of the delivery catheter 100 (for example, the interior volume of the balloon 107 and the inflation lumen 113) may be at least about 1 mL or less, 2 mL, 3 mL, 5 mL, 10 mL, 20 mL, 30 mL, 40 mL, 50 mL, 60 mL, 70 mL, 80 mL, 90 mL, 100 mL, 125 mL, 150 mL, 175 mL, or 200 mL or more.

The length of the balloon 107 may be at least about 0.5 cm, 1 cm, 2 cm, 3 cm, 4 cm, 5 cm, 6 cm, 7 cm, 8 cm, 9 cm, or 10 cm. In some embodiments, the length of the balloon 107 may be configured to accommodate a supported implantable endovascular graft or a supported implantable stent or stent graft or may be configured to span the length of the neuro aneurysm, as described elsewhere herein. In some embodiments, the aneurysm may be relatively small or in an early-stage of development. In some embodiments, the length of the upstream balloon 105 may be the same as the length of the downstream balloon 107 or it may be shorter than the length of the downstream balloon 107. In some embodiments, the length of the upstream balloon 105 may be at least about 0.5 cm, 1 cm, 1.5 cm, 2 cm, 2.5 cm, or 3 cm. In some embodiments, the upstream balloon 105 may comprise a generally spherical shape and the downstream balloon 107 may comprise a generally prolate spheroid shape.

Delivery catheters 100 in which the downstream balloon 107 expands into or is pressed into contact with the peripheral or neuro aneurysm may be particularly suited for aneurysms that are less prone to rupture. In some instances, the risk of rupture may be characterized by the size (for example, maximal diameter) of the aneurysm. Smaller aneurysms (for example, no greater than about 6 cm, 5 cm, 4 cm, or 3 cm) may be less prone to rupture. Neuro aneurysms tend to grow in size over time and become more prone to rupture. The blood vessel wall of the neuro aneurysm may weaken as the aneurysm grows. In some implementations, the delivery catheters 100 described herein, may be particularly useful for early interventional treatment of diagnosed peripheral or neuro aneurysms.

In embodiments comprising an inner balloon 109, the inner balloon 109 may be the same or a different shape as the balloon 107. The inner balloon 109 may comprise an expanded diameter the same as or less than that of the balloon 107. The inner balloon 109 may comprise a length the same as or less than that of the balloon 107. The inner balloon 109 may comprise a maximum interior volume the same as or less than that of the balloon 107. In some embodiments, the volume, length, and/or expanded diameter of the inner balloon 109 may be no less than approximately 100%, 95%, 90%, 85%, 80%, 75%, 70%, 65%, 60%, 55%, 50%, 45%, or 40% of the balloon 107. In embodiments, in which the length of the inner balloon 109 is less than the length of the balloon 107, the inner balloon 109 may be positioned, with respect to the longitudinal axis, centrally within the balloon, or toward the proximal or distal end of the balloon 107. The proximal end of the inner balloon 109 may or may not be aligned with the proximal end of the balloon 107. The distal end of the inner balloon 109 may or may not be aligned with the distal end of the balloon 107.

In some embodiments, the unexpanded diameters of the balloon 105, the balloon 107, and/or the inner balloon 109 of the delivery catheter 100 may be no greater than about 0.5 mm, 1 mm, 2 mm, 3 mm, 4 mm, 5 mm, 6 mm, 7 mm, 8 mm, 9 mm, or 10 mm. The unexpanded diameter of one or more of the balloons may be configured to be received within the lumen of a concentrically surrounding shaft or access sheath.

In some embodiments the weeping balloon (for example, balloon 107) may comprise at least 5, 10, 20, 30, 40, 50, 100, 200, 300, 500, or 1000 pores 126. The diameter (or longest dimension) of the individual pores 126 may be the same or may be different. The diameter of the pores 126 (for example, in an expanded configuration) may be no greater than approximately 0.01 mm, 0.02 mm, 0.03 mm, 0.05 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, or 1 mm. In some embodiments, the diameter of the pores 126 in the expanded configuration may be at least about 1×, 1.25×, 1.5×, 1.75×, 2×, 3×, 4×, 5×, or 10×, the diameter of the pores 126 in the unexpanded configuration. The pores 126 may be the same size regardless the state of expansion in some embodiments, particularly if balloon 107 comprises a non-compliant expandable membrane. In some embodiments, the pores 126 may be disposed over an entire length of the balloon 107. In some embodiments, the pores 126 may be disposed over only about the middle 20%, 30%, 40%, 50%, 60%, 70%, 80%, 90% or 95% of the length of the balloon 107 (for example, in an expanded configuration). In some embodiments, the pores 126 may be disposed only over a distal portion of the length of the balloon 107, the distal portion comprising no more than about 20%, 30%, 40%, 50%, 60%, 70%, 80%, 90% or 95% of the length of the balloon 107 (for example, in an expanded configuration).

In some embodiments, the outer diameter of the main shaft 110 may be no greater than about 1 mm, 2 mm, 3 mm, 4 mm, 5 mm, 6 mm, 7 mm, 8 mm, 9 mm, or 10 mm. In some embodiments, the outer diameter of the main shaft 110 may be approximately 9 Fr, 10 Fr, 11 Fr, 12 Fr, 13 Fr, 14 Fr, 15 Fr, 16 Fr 17 Fr, or 18 Fr. The main shaft 110 may have a sidewall thickness of no greater than approximately 0.1 mm, 0.2 mm, 0.3 mm, 0.4 mm, 0.5 mm, 0.6 mm, 0.7 mm, 0.8 mm, 0.9 mm, 1 mm, 1.25 mm, 1.5 mm, 1.75 mm, or 2.0 mm. The secondary shaft 114 may comprise an outer diameter substantially equal to or slightly less than the inner diameter of the main shaft 110. In some embodiments, the length of the delivery catheter 100 from its proximal end to its distal end 102 may be at least about 20 cm, 25 cm, 30 cm, 35 cm, 40 cm, 45 cm, or 50 cm.

The various components of the delivery catheter 100 may be fabricated from one or more materials known in the art of catheter design. The materials, particularly those configured to be placed in contact with the intravascular environment, may be fabricated from biocompatible materials. In some embodiments, one or more components of the delivery catheter, such as the main shaft 110 and/or secondary shaft 114, may comprise polyurethane (PU), polyethylene (PE), polyvinylchloride (PVC), polytetrafluoroethylene (PTFE), polyvinylidene fluoride (PVDF), other fluoropolymers, polyether block amide (for example, PEBAX® or Vestamid®), nylon, etc. In various embodiments, the shafts and/or balloons may be chemically and/or mechanically treated/processed (for example, plasma etched) or coated to provide biocompatibility or mechanical properties (for example, lubricious and/or hydrophilic surface properties). For example, one or more components of the delivery catheter 100 may be coated with a formulation comprising polyethylene glycol (PEG). The delivery catheter can also be coated with PGG and/or LeGoo®.

In some embodiments, the delivery catheter 100 may comprise a handle at its proximal end. The main shaft 110 of the delivery catheter 100 may extend from a distal end of the handle. The main shaft 110 may continue through the handle and/or be in fluid communication with a channel formed within the handle. The handle may comprise a grip portion for the operator to grasp. The handle may be used to distally advance and/or proximally retract the delivery catheter 100. In embodiments where the delivery catheter 100 is steerable, the handle may comprise one or more controls for steering (for example, bending a distal portion of) the delivery catheter 100, such as by controlling the extension and retraction of one or more pull wires. In some embodiments, the handle may comprise one or more fluid ports in fluid communication with one or more of the internal lumens, such as the first inflation lumen 113 and the secondary inflation lumen 117. The fluid ports may comprise luer-type connectors for connecting to fluid lines, such as for supplying inflation fluid to the delivery catheter 100. In some embodiments, the fluid ports may comprise stopcocks or other valves for regulating fluid flow from a fluid supply source into the handle. The fluid lines may extend to sources of pressurized fluid (for example, inflation fluid) such as a syringe or pump and/or a vacuum source for providing aspiration. In some embodiments, one more fluid ports may be configured to receive a component of the delivery catheter 100. For example, in embodiments, in which the secondary shaft 114 is removable from the main shaft 110, the secondary shaft 114 may be insertable into a proximal end of the handle through the fluid port to be received in the main shaft 110. The secondary shaft 114 may be advanced through the fluid port until it extends distally beyond the main shaft 110. The handle may temporarily fix the relative positioning of the shafts 110, 114, as described elsewhere herein. Similarly, in some embodiments, a guidewire may be insertable into a proximal end of the handle through one or more fluid ports to be received in the first central lumen 112 or the secondary central lumen 116. In some embodiments, in which inflation fluid is supplied by a pump or mechanized syringe, and/or in which aspiration is provided, there may be a controller for controlling flow rate through the internal lumens. The controller may be remote to the handle or coupled to or integral with the handle. The handle may comprise one or more controls for modulating (for example, increasing, decreasing, stopping, and/or starting) the flow rate of the inflation fluid and/or the vacuum pressure supplied to one or more of the internal lumens. In some embodiments, the controls may be remote from the handle (for example, part of a remote controller).

### Methods of Treatment

Some embodiments of the present disclosure include methods of treating an aortic aneurysm with compositions comprising PGG or other therapeutic agents, or by implantation of an endovascular graft comprising PGG and/or LeGoo® or a similar poloxamer gel. Some embodiments of the present disclosure include methods of repairing a leaking endovascular graft by delivering compositions comprising PGG and/or LeGoo® or other therapeutic agents to tissue in a region of the leaking endovascular graft, so as to promote strengthening and/or healing of the tissue surrounding the endovascular graft. Similar methods can be employed for treatments utilizing stents or stent grafts. Some methods include administering a compound, composition, pharmaceutical composition described herein to a subject in need thereof. In some embodiments, a subject can be an animal, for example, a mammal, such as a human. In some embodiments, the subject is a human.

Some embodiments include methods of treating a dissection, e.g., an aortic or thoracic dissection, with compositions comprising PGG and/or LeGoo®, or other therapeutic agents. Some methods include administering a compound, composition, pharmaceutical composition described herein to a subject in need thereof. In some embodiments, a subject can be an animal, for example, a mammal, e.g., a human, a dog, a cat, a pig, a cow, a sheep, a goat, or a horse. In some embodiments, the subject is a human.

Some embodiments include methods of treating a neuro or peripheral aneurysm with compositions comprising PGG or other therapeutic agents. Some methods include administering a compound, composition, pharmaceutical composition described herein to a subject in need thereof. In some embodiments, a subject can be an animal, for example, a mammal, such as a human. In some embodiments, the subject is a human.

Further embodiments include administering a combination of compounds to a subject in need thereof. A combination can include a compound, composition, pharmaceutical composition described herein with an additional medicament.

### Definitions

The term “subject” as used herein, is a broad term, and is to be given its ordinary and customary meaning to a person of ordinary skill in the art (and is not to be limited to a special or customized meaning), and refers without limitation to a human or a non-human mammal, for example, a dog, a cat, a mouse, a rat, a cow, a sheep, a pig, a goat, a non-human primate or a bird, for example, a chicken, as well as any other vertebrate or invertebrate.

The term “mammal” as used herein is a broad term, and is to be given its ordinary and customary meaning to a person of ordinary skill in the art (and is not to be limited to a special or customized meaning), and is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” are broad terms, and are to be given their ordinary and customary meaning to a person of ordinary skill in the art (and are not to be limited to a special or customized meaning), and refer without limitation to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein are broad terms, and are to be given their ordinary and customary meaning to a person of ordinary skill in the art (and are not to be limited to a special or customized meaning), and refer without limitation to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously.

Examples of additional medicaments include collagen crosslinking agents, such as glutaraldehyde, genipin acyl azide, and/or epoxyamine.

Examples of additional medicaments include collagen crosslinking agents, such as glutaraldehyde, genipin acyl azide, and/or epoxyamine. Other drugs include antiplatelet drugs (e.g., aspirin or clopidogrel), blood thinners (e.g., rivaroxaban (Xarelto), apixaban (Eliquis) or edoxaban (Savaysa)), or thrombolytics (e.g., heparin, enoxaparin (Lovenox), dalteparin (Fragmin), fondaparinux (Arixtra), warfarin (Coumadin, Jantoven) or dabigatran (Pradaxa)).

To further illustrate, examples are included. The examples should not, of course, be construed as specifically limiting the invention. Variations of these examples within the scope of the claims are within the purview of one skilled in the art and are considered to fall within the scope of the invention as described and claimed herein. One will recognize that the skilled artisan, armed with the present disclosure, and skill in the art is able to prepare and use the devices and compositions without exhaustive examples.

Although the invention has been described with reference to embodiments and examples, it should be understood that numerous and various modifications can be made without departing from the spirit of the invention. Accordingly, the invention is limited only by the following claims.

It is understood that this disclosure, in many respects, is only illustrative of the numerous alternative device embodiments. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (for example, compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (for example, where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for treating a leaking endovascular graft of a patient, comprising:
positioning a first balloon in an artery in a region of the leaking endovascular graft;
expanding the first balloon such that it presses against surfaces of the artery or the leaking endovascular graft in contact with a surface of the first balloon; and
delivering a therapeutic agent to tissue in the region of the leaking endovascular graft through pores in the first balloon.

2. The method of claim 1, wherein expanding the first balloon comprises introducing an inflation fluid into an interior volume of the first balloon.

3. The method of claim 1, wherein delivering the therapeutic agent comprises introducing a solution comprising the therapeutic agent into an interior volume of the first balloon, the introduction of the solution being configured to expand and/or maintain an expanded state of the first balloon.

4. The method of claim 1, wherein expanding the first balloon comprises maintaining a pressure within an interior volume of the second balloon greater than a diastolic blood pressure of the patient and less than a systolic blood pressure of the patient.

5. The method of claim 1, wherein expanding the first balloon and delivering the therapeutic agent through the pores comprises introducing a solution into an interior volume of the first balloon, and wherein the solution is introduced at a first volumetric flow rate to expand the first balloon and the solution is introduced at a second volumetric flow rate to deliver the therapeutic agent through the pores, the first volumetric flow rate being greater than or equal to the second volumetric flow rate.

6. The method of claim 5, wherein the first volumetric flow rate is greater than the second volumetric flow rate.

7. The method of claim 1, wherein blood flow is occluded by the first balloon no longer than approximately 3 minutes.

8. The method of claim 7, wherein at least 1 mL of solution comprising the therapeutic agent is delivered while downstream blood flow and retrograde blood flow is occluded.

9. The method of claim 1, wherein expanding the first balloon comprises inflating a second balloon disposed within an interior volume of the first balloon.

10. The method of claim 1, wherein delivering the therapeutic agent comprises inflating a second balloon disposed within an interior volume of the first balloon to force a volume of solution comprising the therapeutic agent within the interior volume of the first balloon through the pores.

11. The method of claim 1, wherein the therapeutic agent comprises pentagalloyl glucose (PGG).

12. The method of claim 11, wherein the PGG is at least 99.9% pure.

13. The method of claim 11, wherein the therapeutic agent is substantially free of gallic acid or methyl gallate.

14. The method of claim 1, wherein the therapeutic agent is in admixture with a biocompatible poloxamer gel having reverse thermosensitive properties.

15. A method for treating a leaking endovascular graft of a patient, comprising:
delivering a therapeutic agent to tissue in a region of the leaking endovascular graft, wherein the therapeutic agent comprises at least one of pentagalloyl glucose (PGG) and a biocompatible poloxamer gel having reverse thermosensitive properties.

16. The method of claim 15, wherein the therapeutic agent comprises PGG, and wherein the PGG is at least 99.9% pure.

17. The method of claim 15, wherein the therapeutic agent is substantially free of gallic acid or methyl gallate.

18. The method of claim 15, wherein the therapeutic agent is an admixture of the PGG and the biocompatible poloxamer gel.

19. The method of claim 15, wherein the region is situated behind the leaking endovascular graft.

20. The method of claim 15, wherein the therapeutic agent is delivered by a microcatheter or a weeping balloon.
